(19) United States
(12) Patent Application Publication
(10) Pub. No.: US 2022/0165364 A1
(43) Pub. Date: May 26, 2022

(54) Systems and Methods for Determining Molecular Properties with Atomic-Orbital-Based Features

(71) Applicants: California Institute of Technology, Pasadena, CA (US); Entos, Inc., Los Angeles, CA (US)

(72) Inventors: Zhuoran Qiao, Pasadena, CA (US); Animashree Anandkumar, Pasadena, CA (US); Thomas Francis Miller, South Pasadena, CA (US); Matthew Gregory Welborn, Christiansburg, VA (US); Frederick Roy Manby, Bristol (GB); Feizhi Ding, Hacienda Heights, CA (US); Daniel George Smith, Blacksburg, VA (US); Peter John Bygrave, Bristol (GB); Sai Krishna Sirumalla, Somerville, MA (US); Anders Steen Christensen, Basel (CH)

(73) Assignees: California Institute of Technology, Pasadena, CA (US); Entos, Inc., Los Angeles, CA (US)

(21) Appl. No.: 17/332,951

(22) Filed: May 27, 2021

Related U.S. Application Data
(60) Provisional application No. 63/030,806, filed on May 27, 2020.
(60) Provisional application No. 63/053,192, filed on Jul. 17, 2020.
(60) Provisional application No. 63/190,651, filed on May 19, 2021.
(60) Provisional application No. 63/190,656, filed on May 19, 2021.
(60) Provisional application No. 63/190,657, filed on May 19, 2021.

(51) Int. Cl. G16C 10/00; G16C 20/70

(52) U.S. Cl. CPC G16C 10/00; G16C 20/70

(57) ABSTRACT

Systems and methods for determining molecular structures based on atomic-orbital-based features are described. Atomic-orbital-based features can be utilized in combination with machine-learning methods to predict accurate properties, such as quantum mechanical energy, of molecular systems.

### CROSS-REFERENCE TO RELATED APPLICATIONS

The current application claims the benefit of priority under 35 U.S.C. § 119(e) to U.S. Provisional Patent Application No. 63/030,806 entitled “Deep Learning For Quantum Chemistry And Molecular-Property Prediction Using Symmetry-Adapted Atomic-Orbital Features” filed May 27, 2020, U.S. Provisional Patent Application No. 63/053,192 entitled “Deep Learning For Quantum Chemistry And Molecular-Property Prediction Using Symmetry-Adapted Atomic-Orbital Features” filed Jul. 17, 2020, U.S. Provisional Patent Application No. 63/190,651 entitled “Multi-task Learning for Electronic Structure to Predict and Explore Molecular Potential Energy Surfaces” filed May 19, 2021, U.S. Provisional Patent Application No. 63/190,656 entitled “OrbNet Applications in Density Function Theory for Organic Chemistry” filed May 19, 2021, U.S. Provisional Patent Application No. 63/190,657 entitled “Gauge Equivariant Learning on Atomic Orbitals for Quantum Chemistry” filed on May 19, 2021. The disclosures of U.S. Provisional Patent Application Nos. 63/030,806, 63/053,192, 63/190,651, 63/190,656, and 63/190,657 are hereby incorporated by reference in its entirety for all purposes.

### FIELD OF THE INVENTION

The present invention generally relates to systems and methods to design and synthesize molecules based on molecular system properties; and more particularly to systems and methods that utilize atomic-orbital-based features with deep learning quantum chemistry computing to determine the properties of synthesized chemicals.

### BACKGROUND

Molecular simulations can be helpful to the discovery effort of scientific industry, including solid-state materials, polymers, fine chemicals, and pharmaceuticals. Current approaches employ physics-based methods which solve quantum mechanical equations to describe the behavior of atoms and molecules. While powerful, current methods come at extraordinary computational costs (consuming a sizable fraction of the world's supercomputing resources) and human-time costs (with necessary calculations taking months or longer of wall-clock time). Advances in molecular simulation would broaden its applications in the industrial innovation and development process.

### BRIEF SUMMARY

Systems and methods in accordance with various embodiments of the invention enable the design and/or synthesis of molecules based on molecular system properties. In many embodiments, molecules with specific molecular system properties can be synthesized for a wide range of product development processes such as drug discovery for the pharmaceutical industry, and material design for the chemical, petroleum, battery and electronics industries. Examples of materials synthesized in accordance with various embodiments of the invention include (but are not limited to): catalysts, enzymes, pharmaceuticals, proteins and antibodies, organic electronics, surface coatings, nanomaterials, and organic materials.

Many embodiments predict molecular system properties based on atomic orbital based features using atomic-orbital-based deep learning (OrbNet) processes. In several embodiments, atomic orbital based features include (but are not limited to): atomic orbital (AO) based features, symmetry-adapted atomic orbital (SAAO) based features, derivatives of AO based features, and derivatives of SAAO features. Examples of molecular system properties in accordance with various embodiments of the invention include (but are not limited to): solubility, binding affinity for molecules, binding affinity for protein, redox potential, pKa, electrical conductivity, ionic conductivity, thermal conductivity, light absorption frequency, light absorption intensity, and light absorption efficiency.

In many embodiments, OrbNet processes can allow for at least 1000-fold speed-ups in computational and wall-clock times over existing physics-based quantum mechanical methods. In several embodiments, the processes allow for at least 100-fold increases in human efficiency. By deploying OrbNet at scale with cloud resources, the timescale for turnaround can be reduced from days to seconds. OrbNet in accordance with several embodiments of the invention can enable at least 10-fold prediction accuracy improvements. Some other embodiments implement the software packages, de-risk computational predictions, reduce down-stream experimental and production costs, and accelerate time-to-market.

One embodiment of the invention includes a method of synthesizing a molecule comprising, obtaining a set of atomic orbitals for a molecular system using a computer system; generating a set of atomic-orbital-based features based upon the set of atomic orbitals of the molecular system using the computer system; determining at least one molecular system property based on the set of features using an atomic-orbital-based machine learning (OrbNet) model implemented on the computer system; and when the determined at least one molecular system property satisfies at least one criterion by the computer system, synthesizing the molecular system.

In another embodiment, the set of atomic-orbital-based features comprises an attributed graph representation of atomic-orbital-based features.

In a further embodiment, a node feature of the attributed graph representation corresponds to a diagonal atomic orbital block and an edge feature of the attributed graph representation corresponds to an off-diagonal atomic orbital block.

In still another embodiment, the set of atomic-orbitals comprises symmetry-adapted-atomic-orbitals (SAAOs) and the set of atomic-orbital-based features comprises a set of features based on atomic-orbitals, a set of features based on SAAOs, derivatives of a set of features based on atomic-orbitals or derivatives of a set of features based on SAAOs.

In a yet further embodiment, the molecular system is one of a plurality of candidate molecular systems. In addition, determining when the determined at least one molecular system property satisfies at least one criterion further comprises generating a set of atomic-orbital-based features based upon sets of atomic orbitals for each of the candidate molecular systems; determining at least one molecular system property for each of the candidate molecular systems based on the set of atomic-orbital-based features of each of the candidate molecular systems using the OrbNet model; screening the candidate molecular systems based upon the at least one molecular system property determined for each of the candidate molecular systems; and identifying the molecular system based upon the screening.

A still further embodiment also includes training the OrbNet model to learn relationships between sets of atomic-orbital-based features and molecular system properties using a training dataset describing a plurality of molecular systems and their molecular system properties.

In yet another embodiment, training the OrbNet model to learn relationships between sets of atomic-orbital-based features and molecular system properties further comprises obtaining a set of atomic orbitals for each molecular system in the training dataset of molecular systems; and obtaining a set of atomic-orbital-based features based upon the set of atomic orbitals.

In a further embodiment again, obtaining a set of symmetry-adapted-atomic-orbitals for each molecular system in the training dataset of molecular systems by constructing rotationally invariant symmetry-adapted atomic orbital basis sets; and obtaining a set of symmetry-adapted-atomic-orbital-based features based upon at least the symmetry-adapted-atomic-orbitals.

In a further additional embodiment, obtaining the set of atomic orbitals comprises calculating one mean-field electronic structure selected from the group consisting of Hartree-Fock theory, density functional theory, and a semi-empirical method, and obtaining the set of atomic-orbital-based features comprises calculating one mean-field electronic structure selected from the group consisting of Hartree-Fock theory, density functional theory, and a semi-empirical method.

In a still yet further embodiment, obtaining the set of atomic orbitals comprises parameterizing at least one quantum mechanical operator appeared in the formulation of an electronic structure method selected from the group consisting of Hartree-Fock theory, density functional theory, and a semi-empirical method by a neural network, and obtaining the set of atomic-orbital-based features comprises parameterizing at least one quantum mechanical operator appeared in the formulation of an electronic structure method selected from the group consisting of Hartree-Fock theory, density functional theory, and a semi-empirical method by a neural network.

In another additional embodiment, the neural network comprises a graph neural network, wherein at least one node of the graph neural network corresponds to at least one atom, and at least one edge of the graph neural network corresponds to at least one interatomic interaction.

In another embodiment again, training the OrbNet model and the neural network takes place simultaneously.

In a still yet further embodiment, determining the symmetry-adapted-atomic-orbitals comprises diagonalizing at least one diagonal density-matrix block.

In still yet another embodiment, training the OrbNet model comprises graph neural network.

In another additional embodiment, the graph neural network comprises at least one message passing layer and at least one decoding layer.

In a further embodiment again, the molecular system comprises at least one of atoms, molecular bonds, and molecules formed by atoms and molecular bonds.

In still yet another embodiment, the set of features includes atomic-orbital-based features comprising a physical operator.

In a still further embodiment again, the atomic-orbital-based features further comprise at least one feature selected from the group consisting of: elements from a Fock matrix, elements from a Coulomb matrix, elements from a Hartree-Fock matrix, elements from a density matrix; elements from a core Hamiltonian matrix; and elements from an overlap matrix.

In still another embodiment again, the at least one molecular system property comprises at least one property selected from the group consisting of quantum correlation energy, conformer energy, mean-field energy, single point energy, learning energy, molecular orbital energy, potential energy surface, force, inter-atomic force, vibrational frequency, dipole moment, electronic density, response property, thermal property, excited state energy, excited state force, linear-response excited state energy, linear-response excited state force, and spectrum.

In a still further additional embodiment, the synthesized molecular system comprises at least one molecule selected from the group consisting of a catalyst, an enzyme, a pharmaceutical, a protein, an antibody, a surface coating, a nanomaterial, a semiconductor, and an organic material.

Still another additional embodiment includes a method of screening a set of candidate molecular systems comprising: obtaining a set of atomic orbitals for a plurality of candidate molecular systems using a computer system; generating a set of atomic-orbital-based features for each candidate molecular system based upon sets of atomic orbitals for each of the candidate molecular systems using the computer system; determining at least one molecular system property for each of the candidate molecular systems based on the set of atomic-orbital-based features of each of the candidate molecular systems using an atomic-orbital-based machine learning (OrbNet) model implemented on the computer system; screening the candidate molecular systems to identify at least one molecular system possessing at least one molecular system property that satisfies at least one criterion based upon the at least one molecular system property determined for each of the candidate molecular systems using the computer system; and generating a report describing the at least one molecular system identified during the screening of the candidate molecular systems using the computer system.

A yet further embodiment again includes a method of synthesizing a molecular system using an inverse molecule design process comprising: searching for a set of atomic-orbital-based features having at least one molecular system property predicted by an atomic-orbital-based machine learning (OrbNet) model that satisfies at least one criterion using a computer system, where the OrbNet model is trained to receive a set of features of a molecular system and output an estimate of at least one molecular system property; mapping a located set of atomic-orbital-based features to an identified molecular system using a feature-to-structure map using the computer system, where the feature-to-structure map is trained to map a set of atomic-orbital-based features to a corresponding molecular structure; screening the identified molecular system based upon at least one screening criterion using the computer system; and when the identified molecular system satisfies the at least one screening criterion, synthesizing the identified molecular system.

In yet another embodiment again, searching for a set of atomic-orbital-based features having at least one molecular system property predicted by the OrbNet model that satisfies at least one criterion further comprises using at least one generative model to generate candidate sets of features.

In still another further embodiment, the generative model comprises a graph neural network.

Another further embodiment again includes a method of training an atomic-orbital-based machine learning (OrbNet) model to predict at least one molecular system property from a set of atomic orbitals for a molecular system comprising: obtaining a training dataset of molecular systems and their molecular system properties using a computer system; generating a set of atomic-orbital-based features for each molecular system in the training dataset based upon a set of atomic orbitals for each of the candidate molecular systems using the computer system; training a ML model to learn relationships between the set of atomic-orbital-based features of each molecular system in the training dataset and the molecular system properties of each of the molecular systems in the training dataset using the computer system; and utilizing the OrbNet model to predict at least one molecular system property for a specific molecular system based upon a set of atomic-orbital-based features generated for the specific molecular system based upon a set of atomic orbitals for the specific molecular system.

In another further additional embodiment, obtaining a training dataset of molecular systems and their molecular system properties further comprises: generating a set of atomic-orbital-based features for the specific molecular system based upon a set of atomic orbitals for the specific molecular system using the computer system; retrieving atomic-orbital-based features from a database based upon proximity between a retrieved atomic-orbital-based feature and an atomic-orbital-based feature from the set of atomic-orbital-based features for the specific molecular system; and forming the training dataset using the retrieved molecular systems.

In still yet another further embodiment, training the OrbNet model to learn relationships between the sets of atomic-orbital-based features of each molecular system in the training dataset and the molecular system properties of each of the molecular systems in the training dataset further comprises utilizing a transfer learning process to train an OrbNet model previously trained to determine the relationship between an atomic-orbital-based features of a molecular system and a different set of molecular system properties.

In still another further embodiment again, training the OrbNet model to learn relationships between the sets of atomic-orbital-based features of each molecular system in the training dataset and the molecular system properties of each of the molecular systems in the training dataset further comprises utilizing an online learning process to update a previously trained OrbNet model.

Additional embodiments and features are set forth in part in the description that follows, and in part will become apparent to those skilled in the art upon examination of the specification or may be learned by the practice of the disclosure. A further understanding of the nature and advantages of the present disclosure may be realized by reference to the remaining portions of the specification and the drawings, which forms a part of this disclosure

### BRIEF DESCRIPTION OF THE DRAWINGS

The description will be more fully understood with reference to the following figures, which are presented as exemplary embodiments of the invention and should not be construed as a complete recitation of the scope of the invention. It should be noted that the patent or application file contains at least one drawing executed in color. Copies of this patent or patent application publication with color drawing(s) will be provided by the Office upon request and payment of the necessary fee.

FIG. 1 illustrates an atomic-orbital-based machine learning process in accordance with an embodiment of the invention.

FIG. 2 illustrates a user interface for software that enables determination of molecular structures in accordance with an embodiment of the invention.

FIG. 3 illustrates an architecture of an OrbNet process for AO features in accordance with an embodiment of the invention.

FIG. 4 illustrates a workflow of an OrbNet process for SAAO features and the derivatives of the SAAO features in accordance with an embodiment of the invention.

FIG. 5 illustrates the structure of message passing layers in an OrbNet process for SAAO features and the derivatives of the SAAO features in accordance with an embodiment of the invention.

FIG. 6 conceptually illustrates a database of atomic orbital pairs in accordance with an embodiment of the invention.

FIG. 7 illustrates an OrbNet process for harvesting atomic-orbital-based features in accordance with an embodiment of the invention.

FIG. 8 illustrates an OrbNet process to determine molecular system properties incorporating machine learning regression in accordance with an embodiment of the invention.

FIG. 9A illustrates a process for selecting a candidate molecular system to synthesize, where the process uses an OrbNet model in accordance with an embodiment of the invention.

FIG. 9B illustrates a process for identifying a molecular system to synthesize, where the process uses an inverse molecule design process based upon an ML model in accordance with an embodiment of the invention.

FIG. 9C illustrates an OrbNet process for generating training data relevant to a specific molecular system for the purposes of training an OrbNet model for use in the estimation of at least one chemical property of the specific molecular system in accordance with an embodiment of the invention.

FIG. 10 illustrates a process for querying a database generated using an OrbNet process in accordance with an embodiment of the invention.

FIGS. 11A and 11B illustrate prediction errors for molecule total energies and relative conformer energies respectively using OrbNet processes trained using various datasets in accordance with various embodiments of the invention.

FIG. 12 illustrates comparison of the accuracy and computational cost tradeoff for a range of potential energy methods for the Hutchison conformer benchmark dataset in accordance with an embodiment of the invention.

FIGS. 13A and 13B illustrate the molecular geometry optimization accuracy for the ROT34 and MCONF datasets respectively in accordance with an embodiment of the invention.

FIG. 14 illustrates statistics over the accuracy and coverage of the GMTKN55 dataset using OrbNet Denali processes in accordance with an embodiment of the invention.

FIG. 15 illustrates MAE in kcal/mol for the subsets of the GMTKN55 dataset that are covered by OrbNet Denali training data in accordance with an embodiment of the invention.

FIG. 16 illustrates comparisons between computational cost and the resulting accuracy for the Hutchison conformer benchmark set in accordance with an embodiment of the invention.

FIG. 17 illustrates the OrbNet error relative to the same torsional profiles calculated at the ωB97X-D3/def2-TZVP level of theory of the 25 druglike molecules torsional profiles from the TorionNet500 database in accordance with an embodiment of the invention.

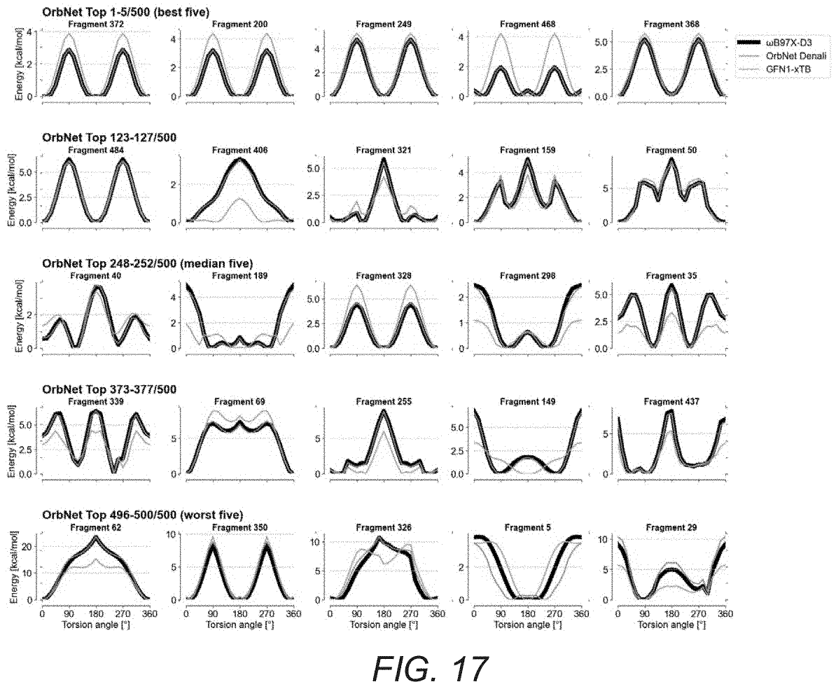

FIG. 18A illustrates the OrbNet prediction of energy on QM9 data set at different training data sizes in accordance with an embodiment of the invention.

FIG. 18B illustrates the OrbNet prediction of dipole moment on QM9 data set at different training data sizes in accordance with an embodiment of the invention.

### DETAILED DESCRIPTION

Turning now to the drawings, systems and methods for synthesizing molecules with specific molecular system properties are described. A molecular system can be atoms, chemical bonds, and/or the resulting molecules formed by the atoms and chemical bonds. Many embodiments implement an atomic-orbital-based deep learning (OrbNet) process to determine properties of a molecular system. In a number of embodiments, an OrbNet model is utilized to perform generative design of molecular systems having particular desirable properties that can then be synthesized.

In several embodiments, specific molecular system properties are utilized as inputs of an OrbNet process. In many embodiments, the input properties of the molecular system are a set of features based on atomic orbitals (AOs) and/or the derivatives of a set of AO features. Some embodiments include the input features can be obtained from low cost and minimal basis mean-field electronic structure methods. In many embodiments, the input properties of the molecular system are a set of features based on symmetry-adapted atomic orbitals (SAAOs) and/or the derivatives of a set of SAAOs features. SAAOs are a set of atom-centered orbitals that satisfies one or more symmetries of the molecular system. SAAOs satisfy translational and rotational symmetry of the molecule, and permutational symmetry of the atoms. In several embodiments, AOs including (but not limited to) SAAOs can be derived from the set and/or a subset of transformed atomic orbital basis for the molecular system and/or other external potential. Certain embodiments provide that the AOs including (but not limited to) SAAOs can be obtained via the reduced density matrix of the molecular system in the atomic orbital representation. In a number of embodiments, the AOs including (but not limited to) SAAOs can be obtained via schemes based on eigenvalues of the Fock matrix in the atom orbital representation and/or the Wigner rotations. Several embodiments provide that AO based features including (but not limited to) SAAO based features can be scalar and/or tensor quantities derived from expectation values of quantum operators and/or the derivatives of expectation values of operators with respect to the AOs. In a number of embodiments, the quantum operators can be the ones in Hartree-Fock theory. Examples of Hartree-Fock operators include (but are not limited to): elements of a Fock (F) matrix, elements of a Coulomb (J) matrix, elements of a Hartree-Fock exchange (K) matrix, elements of a density (P) matrix, elements of an orbital centroid distance (D) matrix, elements of a core Hamiltonian (H) matrix, and/or elements of an overlap (S) matrix. Many embodiments provide that the quantum operators can be based upon Kohn-Sham density functional theory including (but not limited to): the exchange-correlation operator, the exchange-correlation operators' approximations, and the exchange-correlation operators' components. Many embodiments provide that the quantum operators can be in density functional tight binding theory calculation and/or other empirical electronic structure theory methods including (but not limited to): the shell-resolved charges and approximations to the Coulomb, Exchange, Fock, and/or exchange-correlation operators. Several embodiments include quantum operators that can be properties of the molecular systems. Examples of the properties include (but are not limited to): dipole moment, interatomic distance matrix, continuum solvation energy. Many embodiments implement neural networks including (but not limited to) graph neural networks to parameterize matrixes including (but not limited to) a Fock (F) matrix, a Coulomb (J) matrix, a Hartree-Fock exchange (K) matrix, a density (P) matrix, an orbital centroid distance (D) matrix, a core Hamiltonian (H) matrix, and an overlap (S) matrix to generate AO-based features. As can readily be appreciated, the specific AO features used to describe a molecular system in accordance with various embodiments of the invention are largely only limited by the requirements of specific applications. OrbNet processes in accordance with several embodiments of the invention utilize features that are symmetric. As is discussed further below symmetry is not a requirement. OrbNet processes in accordance with many embodiments of the invention utilize features that may or may not be symmetric as appropriate to the requirements of specific applications.

In many embodiments, the OrbNet processes utilize models that are trained using input datasets. Many embodiments predict certain properties of a molecular system as outputs based on relationships between the input AO features including (but not limited to) SAAO features and the properties that are learned during the training of the OrbNet model. OrbNet can predict high quality electronic structure energies in accordance with several embodiments. In some embodiments, the output properties can include (but are not limited to): (1) computable properties of molecules such as solutions of the many body Schrodinger equation including ground and/or excited state mean field energies, ground and/or excited state many body correlation energies, potential energy surfaces, total and/or relative conformer energies, electronic energies, correlation energies, SAAO-pair contributions, mean-field energies, single-point energies, molecular orbital energies, thermal properties, forces, inter-atomic forces, vibrational frequencies (hessian), dipole moments, electron densities, excited state energies, linear-response excited states and forces, and/or spectra; and (2) experimentally measurable properties of molecules such as activity coefficients, solubility, pKa, pH, partition coefficients, vapor pressures, melting points, boiling points, flash points, solvation free energies, redox potential, electrical conductivity, ionic conductivity, thermal conductivity, light absorption frequency, light absorption intensity, light absorption efficiency, viscosity, ADME properties, toxicity, drug toxicity, binding affinity, and/or protein binding affinity. A number of embodiments implement the derivatives of SAAO features as input in OrbNet models and are able to predict response properties including (but not limited to): forces, optimized geometries, inter-atomic forces, dipoles, and linear-response excited states.

In some embodiments, the prediction of forces and/or hessian can be used to optimize the geometry of the molecular system to a local minimum or saddle point. Several embodiments provide that the prediction of forces can be used to run molecular dynamics. In a number of embodiments, the prediction of energies and/or forces can be used to perform configurational sampling. In several embodiments, a molecular system is selected based upon the predicted properties for the molecular system output by the OrbNet model based upon the input AO features including (but not limited to) SAAO features of the molecular system. In a number of embodiments, the OrbNet model can be used to perform generative design in which a search is performed within feature space to identify at least one set of AO features including (but not limited to) SAAO features that provide a desired molecular system property. In several embodiments, AO features including (but not limited to) SAAO features can be mapped to molecular structures using a feature-to-structure map that can be derived from a training data set using a deep learning process. The molecular system(s) corresponding to the identified set(s) of AO features including (but not limited to) SAAO features can then be further analyzed to determine the molecular system(s) most suited to a particular application. As can readily be appreciated, systems and methods in accordance with various embodiments of the invention can utilize any of a variety of input AO features of a molecular system to predict any of a variety of different properties of a corresponding molecular system as appropriate to the requirements of specific applications.

Many embodiments provide that OrbNet processes can predict properties corresponding to a larger and/or a different atomic orbital basis set based on one particular and/or a minimal basis set input. Several embodiments provide that OrbNet processes can predict properties corresponding to a more expensive and/or a different level of electronic structure theory including (but not limited to) density function theory (DFT) with a hybrid exchange-correlation functional based on an input of one level of electronic structure theory including (but not limited to) DFT with a local density approximation or a semi-empirical electronic structure method.

In several embodiments, the molecular systems predicted by the output properties can be in the same molecular family as the input molecular systems. In many embodiments, the molecular systems predicted by the output properties can be in a different molecular family as the input molecular systems. Examples of different molecular families can include (but are not limited to): molecular compositions, molecular geometries, and/or bonding environments. Sets of input AO features including (but not limited to) SAAO features in many embodiments have no explicit dependence on atom types, thus OrbNet processes can enhance chemical transferability of the training results.

In a number of embodiments, the OrbNet processes are implemented as software applications. Several embodiments implement the OrbNet processes in a quantum chemistry software package, which automatically reduces the computational and human-time costs of molecular simulations while leaves the user-interface unchanged. Many embodiments provide that integration of OrbNet into existing industrial workflows can improve calculation speed with no degradation in accuracy and no need for retraining for users.

In many embodiments, more complex models of molecular systems can be utilized including (but not limited to) attributed graph representations of molecular systems, as an alternative to the matrix organized representations. In some embodiments, the topology and connectivity of the graph representation can be derived from the set and/or a subset of the AO feature and/or SAAO feature tensors. In some embodiments, quantum chemical information can be represented as an attributed graph G(V, E, X, X^{e}). In several embodiments, the node features of the attributed graph correspond to diagonal AO blocks including at least a set of AOs, and the edge features correspond to off-diagonal AO blocks including at least a set of AOs. In certain embodiments, the node features of the attributed graph correspond to diagonal SAAO features (Xᵤ=[Fᵤᵤ, Jᵤᵤ, Kᵤᵤ, Pᵤᵤ, Hᵤᵤ]) and the edge features correspond to off-diagonal SAAO features (X^{e}ᵤᵥ=[Fᵤᵥ, Jᵤᵥ, Kᵤᵥ, Dᵤᵥ, Pᵤᵥ, Sᵤᵥ, Hᵤᵥ]). Graph based representations of molecular systems can enable multi-task learning. As can readily be appreciated, appropriately constructed graph representations can provide the benefit of permutation invariance and size extensivity. In many embodiments, a graph neural network (GNN) machine learning architecture including message passing layers can be utilized to perform the machine learning task from the graph-based representations to a diverse set of chemical properties. GNN architecture in accordance with some embodiments can include at least two message passing layers. Several embodiments can include three message passing layers in a GNN architecture. In a number of embodiments, OrbNet processes can utilize graph representations of molecular systems to form general chemical property classification.

In several embodiments, the transferability of OrbNet models is leveraged in machine learning regression processes that utilize pre-trained energy-based models that are transferred to general molecular properties. Several embodiments utilize regression training of graph neural networks (GNNs). GNNs in accordance with some embodiments can include message passing layers and decoding functions. In certain embodiments, the message passing layers can be realized using aggregation functions on hidden node features and edge features. In a number of embodiments, the decoding functions can be realized using summation functions on transformed node attributes. Many embodiments provide that the decoding functions can be realized using graph readout functions including (but not limited to): summation on transformed edge attributes, global graph pooling functions, and Recurrent Neural Networks. OrbNet processes in accordance with many embodiments of the invention can support a broad class of readout functions based on geometric operations. Several embodiments implement multi-task learning in the OrbNet processes to improve learning efficiency. During multi-task learning, OrbNet processes in accordance with some embodiments can be trained with both molecular energies and other computed properties of the quantum mechanical wavefunction in accordance with some embodiments. In several embodiments, OrbNet processes can be trained with experimentally measured quantities including (but not limited to) solvation energies. Furthermore, as increasing amounts of quantum simulation data are generated, OrbNet processes in accordance with many embodiments of the invention can actively update underlying OrbNet models based upon new data without requiring retraining using the original training data corpus.

Many embodiments implement a deep learning architecture in OrbNet processes for learning chemical properties. OrbNet processes in accordance with several embodiments implement quantum-mechanical molecular representation and gauge symmetry. Several embodiments construct molecular representations based on the tight-binding approximated wavefunction and atomic orbitals (AOs). Some embodiments provide that the AOs based molecular representations encode the physics prior better and are infinitely differentiable. In many embodiments, OrbNet processes with AO based features integrate gauge symmetries in quantum interactions by formulating OrbNet as an equivariant map acting on tight-binding quantum operators. In a number of embodiments, OrbNet processes with AO based features implement O(3)-covariant embedding and interaction blocks to parameterize the equivariant map to learn on the basis of AOs and avoid manually fixing the reference system. OrbNet processes with AO based features in accordance with some embodiments take inputs from quantum operators instead of vectors in [symbol] ³, which differs from point-cloud-based equivariant networks. Certain embodiments provide that OrbNet processes with AO based features are equivariant with respect to non-orientation-preserving transformations through tracking the parity of spherical tensors, which may not be properly treated in SE(3) equivariant neural networks. The expressive power limitations present in many equivariant neural networks can be alleviated by normalization schemes, RepNorm in accordance with many embodiments. Several embodiments utilize a RepNorm normalization scheme to obtain more robust learning in OrbNet setups and/or other equivariant networks.

OrbNet processes in accordance with several embodiments of the invention can improve efficiency and accuracy in quantum simulation. In a number of embodiments, the output properties generated from OrbNet processes are transferable and thus can be used to determine molecules of different molecular systems. In some embodiments, OrbNet processes possess transferability across molecular geometries. Several embodiments implement OrbNet processes with transferability within a molecular family. Some embodiments implement OrbNet processes providing transferability across bonding environments. Certain embodiments implement OrbNet processes providing transferability across chemical elements. In several embodiment, OrbNet provides about 33% improvement in prediction accuracy with the same amount of data. In many embodiments, OrbNet processes provide a prediction accuracy similar to DFT, but at a computational cost that is reduced by at least three orders of magnitude relative to DFT methods.

Many embodiments implement chemical transferability of OrbNet processes across molecular systems and so are capable of identifying molecules with a broad range of properties. Molecules with specific molecular system properties can be synthesized using processes in accordance with various embodiments of the invention for a wide range of product development processes such as drug discovery and material design. Examples of such embodiments include (but are not limited to) processes that can be utilized for: organic light emitting diode material design, catalyst design, enzyme reactions and drug design, protein and antibody design, organic material design, nanomaterial design, and/or material design for the battery, chemical, and petroleum industries.

Systems and methods for implementing OrbNet processes in accordance with various embodiments of the invention are discussed in further detail below.

### Machine Learning in Quantum Chemistry

Machine learning for molecules mostly encodes the molecular system as graphs or point clouds, while lacking fundamental information on its quantum interactions. At a fundamental level, chemistry can be described by the Born-Oppenheimer many-body Schrödinger equation:

ĤΨ(rₑ;R)=E(R)Ψ(rₑ;R) (1)

where Ψ(rₑ; R) is the wavefunction at electron positions rₑ and atom nuclei positions R, and E(R) is the molecular system's energy. Conceptually Eq. 1 may be used to simulate chemical reactions, but quantum correlation makes it an intractable O(N!) problem to solve. Approximate numerical methods such as density functional theory (DFT) can suffer from a punitive scaling and speed-accuracy tradeoffs, which may be impractical for large-scale applications such as drug discovery. The potential energy surface is a central quantity of interest in the modelling of molecules and materials. Calculation of these energies with sufficient accuracy in chemical, biological, and materials systems can be adequately described at the level of DFT. However, due to its relatively high cost, the applicability of DFT is limited to either relatively small molecules or modest conformational sampling, at least in comparison to force-field and semi-empirical quantum mechanical theories. A major focus of machine learning (ML) for quantum chemistry has been to improve the efficiency with which potential energies of molecular and materials systems can be predicted while preserving accuracy. Despite the success of such methods in predicting energies on various benchmarks, the generalizability of deep neural network models across chemical space and for out-of-equilibrium geometries is less investigated. The quantities derived from stationary solutions of Eq. 1, e.g. E(R), may be learned to address this challenge.

The problem of empirically approximating E(R) has been known as determining a molecule's force field. While constructing a force field requires extensive domain expertise on engineering its functional form, machine-learning approaches have been proposed to approximate E(R) from data with higher flexibility, using either hand-crafted features or graph neural networks based on distance information and more recently with generalized geometric information. Such empirical approaches, however, regard the molecule as a (classical) point cloud of atom nuclei coordinates (R in Ψ(rₑ; R)), thus are unaware of the quantum-mechanical interactions carried by the electrons (rₑ in Ψ(rₑ; R)). On the other hand, previous works that focus on constructing molecular representations with quantum-mechanics signatures have shown promising accuracy on certain tasks, but they mostly require a numerical calculation cost similar to DFT for obtaining the representation, and some may require compute-intensive feature processing to enforce symmetry.

Previous work in quantum chemistry has focused on predicting electronic energies or densities based on atom- or geometry-specific features, and kernel-based or neural-network machine learning architectures. (See, e.g., J. S. Smith, et al., Chem. Sci, 2017, 8, 3192-3203; L. Zhang, et al., Phys. Rev. Lett., 2018, 120, 143001; M. Rupp, et al., Phys. Rev. Lett., 2012, 108, 58301; K. Hansen, et al., J. Chem. Theory Comput., 2013, 9, 3404; the disclosures of which are incorporated herein by references in their entirety.) Recent studies have focused on the featurization of molecules in abstracted representations, such as quantum mechanical properties obtained from low-cost electronic structure calculations, and the utilization of graph-based neural network techniques to improve transferability and learning efficiency. (See, e.g., M. Welborn, et al., J. Chem. Theory Comput., 2018, 14, 4772-4779; L. Cheng, et al., J. Chem. Phys., 2019, 150, 131103; K. Yang, et al., J. Chem. Inf. Model, 2019, 59, 3370-3388; J. Klicpera, et al., International Conference on Learning Representations, 2020; the disclosures of which are incorporated herein by references in their entirety.)

Several ML methods have been developed for the prediction of high-level (i.e., coupled-cluster) correlation energies based on quantum mechanical features from a mean-field-level (i.e., HF theory or DFT) electronic structure calculation. U.S. Patent Application No. 2020/0294630 to Miller et al. describes a molecular-orbital-based machine learning (MOB-ML) approach to the prediction of molecular properties using localized molecular orbitals for input feature generation, with applications that include the prediction of correlated wavefunction properties based on the information from a mean-field reference theory.

In MOB-ML, localized molecular orbitals are obtained via an orbital localization procedure (such as Boys, IBO, etc.), with the orbitals obtained from a mean-field electronic structure calculation. Feature vectors are then calculated for diagonal and off-diagonal molecular orbital pairs from matrix elements of the molecular orbitals with respect to various operators (i.e., Fock, Coulomb, and exchange operators) within the basis and using a feature sorting scheme. Gaussian-Process or clustering-based regressors are trained for the pair correlation energy labels associated to the MOB feature vectors. In contrast, OrbNet processes in accordance with many embodiments use the AOs for evaluating matrix elements of the operators for feature generation, and employ a GNN scheme for performing regression of AO-resolved properties including (but not limited to) SAAO-resolved properties (such as the SAAO-pair contributions to the correlation energy), whole molecule properties including (but not limited to) drug toxicity, binding affinity, pKa, correlation energy, mean-field energy, atom-resolved properties including (but not limited to) partial charges, Fukui reactivity, proton affinity, and/or bond-resolved properties including (but not limited to) bond dissociation energies, bond orders.

In MOB-ML, LMOs are generated using an iterative orbital localization procedure which can include a series of O(N³) operations, therefore hindering the efficiency of feature generation with semi-empirical methods and on large molecular systems. In many embodiments, OrbNet processes allow for using approximate quantum-mechanical models 1000 times faster than DFT to build the representation and formulate physical symmetry into neural network architecture design. OrbNet processes in accordance with several embodiments use SAAOs for featurization which can be obtained within a one-shot O(N) block-diagonalization operation, resolving the computational bottleneck when an inexpensive electronic structure method is employed for feature generation. In comparison, many embodiments provide that OrbNet processes using AOs for featurization can perform faster by eliminating the one-shot O(N) block-diagonalization operation.

NeuralXC (See, e.g., S. Dick, et al., Machine Learning Accurate Exchange and Correlation Functionals of the Electronic Density, 2019; the disclosure of which is incorporated herein by reference in its entirety) and DeePHF (See, e.g., Y. Chen, et al., Ground State Energy Functional with Hartree-Fock Efficiency and Chemical Accuracy, 2020; the disclosure of which is incorporated herein by reference in its entirety) are machine-learning techniques that employ AO-based features obtained from electronic structure calculations to perform the regression and prediction of molecular energies. Both NeuralXC and DeePHF rely on the electronic density and orbitals obtained from either a Hartree-Fock (HF) (in DeePHF) or low-level density functional theory (DFT) (in NeuralXC) calculation using cc-pVDZ or larger atomic-orbital basis sets. Both models learn the residual terms between the low-level calculation and high-level (such as, CCSD(T)) reference energies. Both models may need the same (or larger) AO basis set for the mean-field calculation than that associated with the high-level (such as, CCSD(T)) prediction. Neither NeuralXC nor DeePHF allows for prediction of large-AO-basis-set results on features obtained directly from minimal-AO-basis mean-field calculations. In contrast, OrbNet processes in accordance with many embodiments allow for the use of minimal-AO-basis calculations (at great reduction in computational cost) for the feature generation. In several embodiments, OrbNet processes include the use of AO basis sets other than minimal basis sets, with and without projection into other basis sets or orbital subspaces, which remains distinct from DeePHF with regard to the manner in which features are constructed.

In both NeuralXC and DeePHF, sets of AOs or quasi-AOs are employed for generating features for machine learning. NeuralXC does not featurize the interactions between different atoms or different quantum-number (principal or angular) shells within atoms. For example, NeuralXC uses the diagonal elements of the density matrix from the mean-field (DFT) calculation in building features. DeePHF also uses diagonal elements of the density matrix from the mean-field (HF) calculation in building features, and in some cases includes interactions between quantities on different atoms. DeePHF does not include interactions between different shells on the same atom, and it introduces the need for a pre-determined weighting function based on inter-atomic distances.

In contrast, OrbNet processes can be more information-rich by construction compared to the existing schemes. Unlike NeuralXC, shell averaging need not be performed in OrbNet processes. Moreover, in contrast to both NeuralXC and DeePHF, some embodiments provide that OrbNet processes include all off-diagonal operator matrix elements (including intra- and inter-atom elements, and intra- and inter-shell) within the features, thereby preserving the information content and enabling description of long-range contributions. In comparison to DeePHF, OrbNet processes in accordance with certain embodiments of the invention can include interactions between different shells on the same atom and avoid the need for a pre-determined weighting function based on inter-atomic distances. In a number of embodiments, OrbNet processes include quantum-chemical matrices including Fock (F) matrix, Coulomb (J) matrix, exchange (K) matrix, density matrix, core Hamiltonian (H) matrix, and/or overlap (S) matrix, which can be important components for energy prediction tasks. Both NeuralXC and DeePHF methods have not been applied for the prediction of DFT-quality results on the basis of lower-level semi-empirical methods, such as GFN-xTB.

Other differences arise in the way how rotational invariance is enforced within the features. In NeuralXC, the rotational invariance of the features can be guaranteed by summing all sub-shell components of the AO-projected density d^{(nl)}=Σₘ₌₋ₗ^{l}Cₙₗₘ² (such as, the trace of the local density matrix), such that the information content is not preserved. In DeePHF, the rotational invariance of the features can be enforced by using the eigenvalues of the local density matrix instead of the trace to build the feature vector for each shell. By contrast, many embodiments provide that OrbNet processes can achieve the rotational invariance of features through the use of SAAOs or through the use of AOs combined with a rotationally equivariant neural network architecture, which involves no loss of information content.

Many embodiments provide that OrbNet processes implement different machine learning methods from NeuralXC and DeePHF. For NeuralXC, the machine learning regression is performed using a Behler-Parrinello type neural network, with the labels associated with a one-body summation over the shells to yield the total energy difference between the level of theory used for the features and the level of theory used for the prediction, i.e., E^{CCSD(T)}−E^{PBE} where PBE refers to the Perdew-Burke-Ernzerhof density functional. For DeePHF, the ML regression is performed using a dense neural network, with the labels associated with a one-body summation over the shells to yield the total correlation energy.

In contrast, OrbNet processes in accordance with many embodiments use a GNN for the machine learning regression. Certain embodiments provide the results using a multi-head graph attention mechanism and/or a performer attention mechanism and residual blocks that greatly improve the representation capacity of the model, to learn complex chemical environments. Unlike the pre-tuned aggregation coefficients in DeePHF, OrbNet processes also offer a flexible framework for learning orbital interactions and could be naturally transferred to downstream tasks.

Several embodiments provide that OrbNet processes possess better inference and training efficiency compared to NeuralXC and DeePHF. In NeuralXC and DeePHF, a large-basis-set SCF calculation may be required to obtain high-fidelity feature values. OrbNet processes in accordance with some embodiments may require only a minimal basis for SCF to reach chemical accuracy for prediction, which can lead to about 100 times to about 1000 times speedup for feature generation.

In many embodiments, OrbNet processes can provide accurate prediction of correlation energies using input features from minimal-basis HF calculations. Some embodiments provide that the OrbNet methods can be about 10-fold more accurate than DeePHF for the prediction of CCSD(T) correlation energies given the same amount of training data.

In several embodiments, OrbNet processes can provide better transferability than DeePHF and NeuralXC. For DeePHF, transferability across diverse organic molecules (the QM7b-T dataset) shows much lower prediction accuracy compared to the OrbNet processes in accordance with embodiments. When trained on 7-heavy-atom organic molecules (the QM7b-T dataset) and tested on larger 13-heavy-atom organic molecules (the GDB13-T dataset), the OrbNet processes exhibit better prediction accuracy than DeePHF and NeuralXC and provide great transferability.

Systems and methods for synthesizing molecules with specific molecular system properties and atomic-orbital-based machine learning (OrbNet) processes that can be utilized in the design and/or synthesis of molecules in accordance with various embodiments of the invention are discussed further below.

### Atomic-Orbital-Based Machine Learning Processes

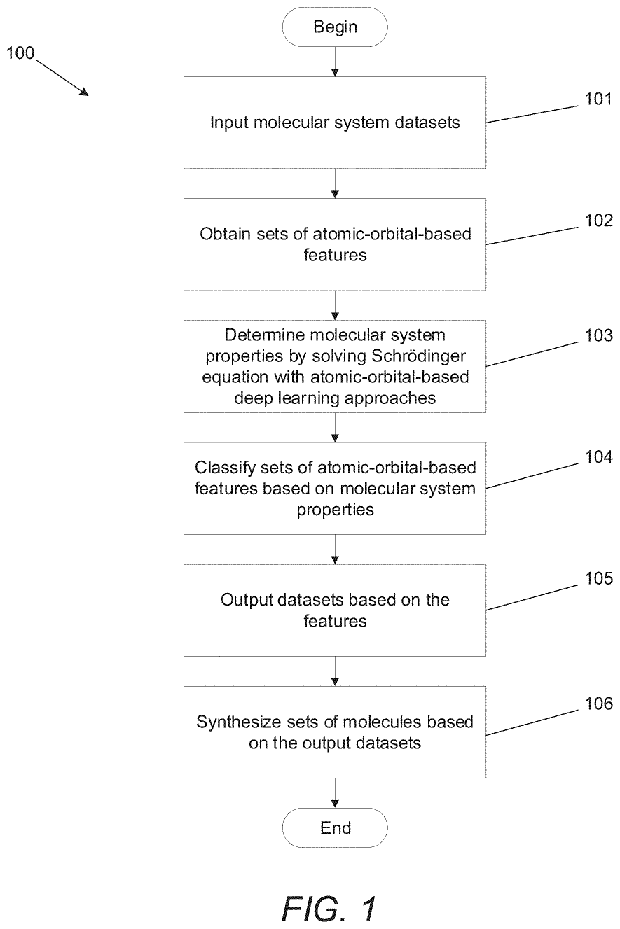

Many embodiments utilize accurate and transferable OrbNet processes to predict properties including (but not limited to) correlated wavefunction energies based on input features using computations including (but not limited to) a self-consistent field calculation. A method for synthesizing molecules using an OrbNet process in accordance with an embodiment of the invention is illustrated in FIG. 1. The process 100 can begin by obtaining a molecular system dataset (101). Some embodiments include input datasets that include molecules with the same elements. In a number of embodiments, input datasets can include molecules with different types of molecular bonds. In several embodiments, input datasets can include molecules with different geometries. Some embodiments include input datasets that include different compositions of the same elements. In many embodiments, datasets can include different molecules and elements. As can readily be appreciated, any of a variety of input datasets can be utilized as appropriate to the requirements of specific applications in accordance with various embodiments of the invention.

Sets of atomic-orbital based (AO-based) features for the input datasets can be obtained based on atomic orbitals (102). In several embodiments, the AO based features include (but are not limited to) a set of features based on AOs, a set of features based on (but not limited to) symmetry adapted atomic orbitals (SAAOs), derivatives of a set of AOs, and/or derivatives of a set of SAAOs. In some embodiments, the AO features can include (but are not limited to) quantum operators of the molecular systems. In several embodiments, input AO-based features can include (but are not limited to): elements of a Fock (F) matrix, elements of a Coulomb (J) matrix, elements of a Hartree-Fock exchange (K) matrix, elements of a density (P) matrix, elements of an orbital centroid distance (D) matrix, elements of a core Hamiltonian (H) matrix, and/or elements of an overlap (S) matrix. Many embodiments provide the quantum operators can be computed with Kohn-Sham density functional theory including (but not limited to): the exchange-correlation operator, the exchange-correlation operators' approximations, and the exchange-correlation operators' components. Several embodiments provide that the quantum operators can be computed with density functional tight binding theory calculation and/or other semi-empirical electronic structure theory methods (e.g. GFN1-xTB) including (but not limited to): the shell-resolved charges and approximations to the J, K, F, P, D, H, S and/or exchange-correlation operators. Several embodiments include that quantum operators can be properties of the molecular systems. Examples of the properties include (but are not limited to): dipole moment, interatomic distance matrix, and/or continuum solvation energy. Many embodiments implement neural networks including (but not limited to) graph neural networks to parameterize matrixes including (but not limited to) a Fock (F) matrix, a Coulomb (J) matrix, a Hartree-Fock exchange (K) matrix, a density (P) matrix, an orbital centroid distance (D) matrix, a core Hamiltonian (H) matrix, and an overlap (S) matrix to generate AO-based features. As can readily be appreciated, any of a variety of input AO based features can be utilized as appropriate to the requirements of specific applications.

In certain embodiments, quantum chemistry calculations are performed using OrbNet processes (103). In a number of embodiments, the computations can be performed on a local computing device. In several embodiments, the calculations are performed on a remote server system. OrbNet processes can be trained with AO-based features of the input datasets.

During a training process (not shown) OrbNet processes can learn relationships between AO-based features and properties of molecular systems using a training dataset. In some embodiments, the training datasets can be subsets randomly selected from input datasets. Examples of molecular datasets in such embodiments can include (but are not limited to): QM7b, QM7b-T, QM9, GDB-13, GDB-13-T, DrugBank, DrugBank-T, ChEMBL27, JSCH-2005, sidechain-sidechain interaction subset of the BioFragment database, MD17, and BfDB-SSI. In several embodiments, the training datasets can be sets of molecules from the same or different molecular systems. As can readily be appreciated, any of a variety of training datasets can be utilized as appropriate to the requirements of specific applications in accordance with various embodiments of the invention.

The OrbNet processes can utilize a trained model that describes relationships between AO based features and properties of molecular systems to perform a ranking and/or categorization (104) of at least the molecules in the input dataset. In many embodiments, the OrbNet processes can also identify novel molecules and/or molecules that are not in the input dataset based upon regions of the feature space that contain molecules that the model predicts will have desirable properties. The various ways in which OrbNet processes can be utilized to identify molecular systems having desirable properties in accordance with various embodiments of the invention including specific examples are discussed further below.

In many embodiments, the trained OrbNet processes generate output datasets of molecular system properties (105). The molecular system properties can include (but are not limited to): (1) computable properties of molecules such as solutions of the many-body Schrodinger equation including ground and/or excited state mean field energies, ground and/or excited state many body correlation energies, potential energy surfaces, total and/or relative conformer energies, electronic energies, correlation energies, AO-pair and/or SAAO-pair contributions, mean-field energies, single-point energies, molecular orbital energies, thermal properties, forces, inter-atomic forces, vibrational frequencies (hessian), dipole moments, electron densities, excited state energies, linear-response excited states and forces, electronic spectra, rotational spectra, nuclear resonance spectra, and/or vibrational spectra; and (2) experimentally measurable properties of molecules such as activity coefficients, solubility, pKa, pH, partition coefficients, vapor pressures, melting, boiling, and flash points, solvation free energies, redox potential, electrical conductivity, ionic conductivity, thermal conductivity, light absorption frequency, light absorption intensity, light absorption efficiency, viscosity, ADME properties, toxicity, drug toxicity, binding affinity, and/or protein binding affinity. A number of embodiments implement the derivatives of AO and/or SAAO features as input and are able to predict response properties including (but not limited to): forces, optimized geometries, inter-atomic forces, dipoles, and/or linear-response excited states. As can readily be appreciated, the specific features used as molecular system properties are largely only limited by the requirements of specific applications. Based on the output datasets, molecules with sets of desired molecular system properties can be identified and synthesized (106).

While various processes for synthesizing chemicals using OrbNet processes are described above with reference to FIG. 1, any of a variety of processes that utilize machine learning to estimate the properties of molecular systems can be utilized in the design and/or synthesis of chemicals as appropriate to the requirements of specific applications in accordance with various embodiments of the invention. For example, molecular systems can be synthesized in a process that utilizes a generative OrbNet process to identify the molecular system as having molecular properties satisfying certain criteria using techniques similar to those discussed below. Processes for designing molecules with desired properties in accordance with various embodiments of the invention are discussed further below.

### Determining Molecular Structures

In many embodiments, OrbNet processes enable real-time chemical modeling, and design, and provides a platform that can be utilized to perform these activities in a collaborative manner. In several embodiments, the OrbNet processes are implemented in software packages that can execute on a local computer or on a remote server. Additionally, the software packages according to some embodiments, can perform calculations on many possible chemical modifications and return rank-ordered recommendations for the most promising chemical modifications. With parallel computation all of the results can be returned in seconds. In this way, processes similar to the various processes for designing molecular systems described above can be performed and the results used to generate intuitive and interactive graphical user interfaces that enable any of a variety of experimental chemists to utilize OrbNet in the design and/or synthesis of chemicals.

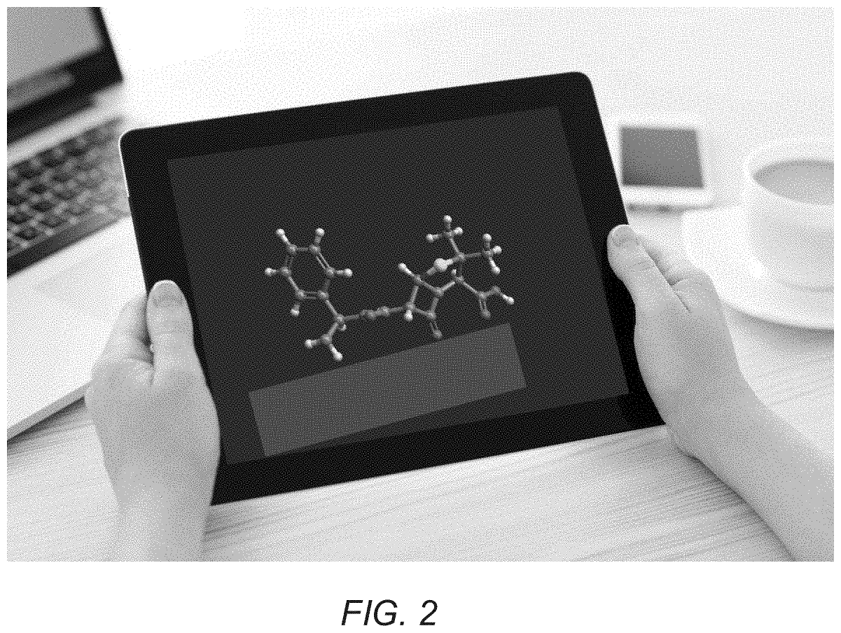

A user interface that can be generated by software using a ML process implemented in accordance with an embodiment of the invention is conceptually illustrated in FIG. 2. In many embodiments, the software can enable any experimental chemist, instead of only expert computational chemists, to identify molecular systems possessing desirable chemical properties. For example, user interfaces can be implemented for the software that can enable the design and synthesis of molecular systems by any of a variety of experimental chemists including (but are not limited to): medicinal chemists, synthetic chemists, material scientists, and/or biochemists.

While various processes for designing molecules using OrbNet processes are described above with reference to FIG. 2, any of a variety of processes that utilize machine learning to estimate the properties of molecular systems can be utilized in the design and synthesis of chemicals as appropriate to the requirements of specific applications in accordance with various embodiments of the invention. Processes for performing AO based feature generation in accordance with various embodiments of the invention are discussed further below.

### Atomic Orbital Representation for Molecules

The Schrödinger equation (Eq. 1) can be used to simulate chemical reactions, but quantum correlation may make it an intractable problem to solve. Approximate numerical methods such as DFT can suffer from a punitive scaling and speed-accuracy tradeoffs, which may be impractical for large-scale applications. The quantities derived from stationary solutions of Eq. 1, e.g. E(R), may be learned to address this challenge. The problem of empirically approximating E(R) has been known as determining a molecule's force field. While constructing a force field may require extensive domain expertise on engineering its functional form, machine-learning approaches have been proposed to approximate E(R) from data with higher flexibility, using either hand-crafted features or graph neural networks based on distance information and more recently with generalized geometric information. The empirical approaches regard the molecule as a (classical) point cloud of atom nuclei coordinates (R in Ψ(rₑ; R)), and are unaware of the quantum-mechanical interactions carried by the electrons (rₑ in Ψ(rₑ; R)). On the other hand, the work that focuses on constructing molecular representations with quantum-mechanics signatures may require a numerical calculation cost similar to DFT for obtaining the representation, and some may require compute-intensive feature processing to enforce symmetry.

Equivariance has been proposed as a unifying concept for deep learning in the presence of a symmetry prior. With ‘baked-in’ symmetry, equivariant neural networks have been introduced for homogeneous grid and Euclidean data, and can be generalized to gauge-symmetries for manifolds in the contexts of geometrical or mesh-based observations and high-energy physics problems. Some approaches can be applied to molecular modelling, focusing on the 3D rotational symmetry; while the architectures are designed for classical point-cloud-based molecular representations.

Many embodiments implement a deep learning architecture in OrbNet processes for learning chemical properties with quantum-mechanical molecular representation and gauge symmetry. Several embodiments construct molecular representations based on the tight-binding approximated wavefunction and atomic orbitals which better encode the physics prior and are infinitely differentiable. Many embodiments implement gauge-equivariance for quantum operators represented in atomic orbitals. OrbNet processes with AO-based features implement O(3)-covariant embedding and interaction blocks to parameterize the equivariant map to learn on the basis of atomic orbitals and avoid manually fixing the reference system. OrbNet processes with AO-based features in accordance with some embodiments of the invention take inputs from quantum operators instead of vectors in [symbol] ³, which differs from point-cloud-based equivariant networks. Certain embodiments provide that OrbNet processes with AO-based features are equivariant with respect to non-orientation-preserving transformations through tracking the parity of spherical tensors, which may not be properly treated in SE(3) equivariant neural networks. The expressive power limitations present in many equivariant neural networks can be alleviated by a normalization scheme, such as (but not limited to) RepNorm which is utilized in OrbNet processes in accordance with many embodiments of the invention. Several embodiments provide that the RepNorm normalization scheme can give rise to more robust learning in OrbNet setups and be applied to other equivariant networks.

Instead of directly learning E(R) solely relying on the nuclear position information R, several embodiments parameterize a functional [symbol] _{θ} that learns the target property y from an approximate wavefunction Ψ₀(rₑ;R) that can be obtained at low computational cost:

y_{θ}= [symbol] _{θ}[Ψ₀(rₑ;R)] (2)

For molecular systems, Ψ₀(rₑ;R) can be represented in atomic orbitals and quantum operators. Formal notations to intersect with the symbolic conventions employed in quantum mechanics are provided.

Definition of Dirac's Bra-Kets: by letting V be a Hilbert space over [symbol] , for u,v∈V, their Hermitian inner product is denoted by [symbol] u|v [symbol] . |v [symbol] is a ket and [symbol] u| is a bra. When V is a finite dimensional vector space, a bra [symbol] u| can be a row vector and a ket |v [symbol] as a column vector. In physics, |v [symbol] can be referred to as a quantum state. Single-electron quantum states can be used in the real space [symbol] ³, where the Hilbert space is the function space of square-integrable functions: V=L²( [symbol] ³). The inner product is given by [symbol] u|v [symbol] = [symbol] u*(r)v(r)dr, where u*(r) denotes the complex conjugation of u(r).

An atomic orbital |Φ_{A}^{n,l,m} [symbol] takes the functional form

$${Φ}_{A}^{n , l , m} ( r ) := {R}_{n , l}^{{z}_{A}} ( ‖ r - {R}_{A} ‖ ) {Y}_{lm} ( \frac{r - {R}_{A}}{‖ r - {R}_{A} ‖} )$$

where R_{A} is the nuclei position of atom A, z_{A} denotes the atomic number of atom A, R_{n,l}^{z}^{_{A}} is called a radial function which does not depend on the direction of r−R_{A}, and Yₗₘ is a spherical harmonic of rank l and degree m. In quantum mechanics, the indices n, l, m are principal, angular, and magnetic quantum numbers. For hydrogen-like atoms, Φ_{A}^{n,l,m}(r) with certain forms of R_{n,l}^{z}^{_{A}}(r) are known as the exact wavefunction solution of Eq. 1, and for molecular systems they can be employed as the basis functions to numerically represent the many-electron wavefunction. In most cases, the collection of atomic orbitals is neither mutually orthogonal nor a complete basis of V, but serves as a computationally tractable representation basis of Ψ(rₑ;R).

Definition of quantum operators: a (single-electron-reduced) quantum operator [symbol] : V→V is a self-adjoint linear operator defined on V=L²( [symbol] ³). [symbol] |u [symbol] denotes the quantum operator acting on a ket vector. Given a set of kets {|ϕᵢ [symbol] }, Oᵢⱼ:= [symbol] ϕᵢ| [symbol] |ϕⱼ [symbol] is a matrix representation of [symbol] . For the set of kets given by atomic orbitals {|Φ_{A}^{n,l,m} [symbol] } of a molecule, the short-hand notation [symbol] Φ| [symbol] |Φ [symbol] is used to denote the matrix representation of [symbol] in {|Φ_{A}^{n,l,m} [symbol] }.

The molecule can be represented by [symbol] Φ| [symbol] _{Ψ}_{₀}|Φ [symbol] , the matrix representation of quantum operators [symbol] _{Ψ}_{₀} built from an approximate wavefunction Ψ₀(rₑ;R) in the basis of atomic orbitals {|Φ_{A}^{n,l,m} [symbol] } of the given molecule. [symbol] _{θ} can be constructed as [symbol] _{θ}[Ψ₀]= [symbol] _{θ}[ [symbol] Φ| [symbol] _{Ψ}_{₀}|Φ [symbol] ] mapping [symbol] _{Ψ}_{₀} represented in atomic orbitals to the target y, and θ can be determined from data. [symbol] Ψ| [symbol] _{Ψ}_{₀}|Φ [symbol] is an AO-molecular representation serving as the input signals to [symbol] _{θ}. The approximate quantum operators [symbol] _{Ψ}_{₀} can be efficiently computed via a tight-binding Hamiltonian. The required computational time to generate [symbol] _{Ψ}_{₀} is at least 1000-fold lower than obtaining the ground-truth (e.g. a DFT calculation), and [symbol] Φ| [symbol] _{Ψ}_{₀}|Φ [symbol] is infinitely differentiable.

Equivariants in AO molecular representations are provided. Given a transformation g, a map f is said to be equivariant if g∘f=f∘g. Constructing [symbol] _{θ} to correctly describe the physical symmetries in the molecular system may require the map [symbol] _{θ} to be equivariant under certain gauge transformations [symbol] . Gauge transformations [symbol] on an AO-molecular representation [symbol] Φ| [symbol] |Φ [symbol] are composed from translation [symbol] , rotation [symbol] , inversion [symbol] applied to the atom coordinates R, orbital phase transformation [symbol] applied to Ψ₀, and local gauge transformations g_{A} applied to |Φ_{A} [symbol] . It can be shown that the AO-molecular representations [symbol] Φ| [symbol] |Φ [symbol] are by construction invariant to [symbol] and g_{A} without loss of information content, but the global gauge transformations generated by [symbol] , i.e. O(3) need to be explicitly treated within the formulation of [symbol] . The action of [symbol] and [symbol] on [symbol] Φ| [symbol] |Φ [symbol] can be obtained based on group representation theory to address the latter O(3) symmetry.

Lemma 1 O(3) acting on an AO molecular representation is provided. For the global rotation [symbol] and the global inversion [symbol] , the actions on [symbol] Φ| [symbol] |Φ [symbol] is given by

$$\begin{matrix}ℛ [ 〈 {Φ}_{A}^{{n}_{1} , {l}_{1} , {m}_{1}} \} \vert \hat{𝒪} \vert {Φ}_{B}^{{n}_{2} , {l}_{2} , {m}_{2}} 〉 ] = {\sum }_{{m}_{1}^{′} , {m}_{2}^{′}} {{D}^{*} ( ℛ )}_{{m}_{1}^{′} , {m}_{1}^{′}}^{{l}_{1}} 〈 {Φ}_{A}^{{n}_{1} , {l}_{1} , {m}_{1}^{′}} \vert \hat{𝒪} \vert {Φ}_{B}^{{n}_{2} , {l}_{2} , {m}_{2}^{′}} 〉 {D ( ℛ )}_{{m}_{2}^{′} , {m}_{2}}^{{l}_{2}} & ( 3 ) \\ [ 〈 {Φ}_{A}^{{n}_{1} , {l}_{1} , {m}_{1}} \} \vert \hat{𝒪} \vert {Φ}_{B}^{{n}_{2} , {l}_{2} , {m}_{2}} 〉 ] = {( - 1 )}^{{l}_{1} + {l}_{2}} 〈 {Φ}_{A}^{{n}_{1} , {l}_{1} , {m}_{1}^{′}} \vert \hat{𝒪} \vert {Φ}_{B}^{{n}_{2} , {l}_{2} , {m}_{2}^{′}} 〉 & ( 4 )\end{matrix}$$

where A, B are both atom indices, and D^{l}( [symbol] ) are called Wigner-D matrices known for transforming spherical harmonics Yₗₘ given a rotation [symbol] .

### Learning Atomic Orbital Interactions

Several embodiments provide the construction of the gauge-equivariant map [symbol] _{θ}. Some embodiments implement O(3)-covariant neural network layers acting on an AO-molecular representation [symbol] Φ| [symbol] |Φ [symbol] . In some embodiments, the ‘local’ blocks can be O_{AA}:= [symbol] Φ_{A}| [symbol] |Φ_{A} [symbol] and ‘non-local’ blocks can be O_{AB}:= [symbol] Φ_{A}| [symbol] |Φ_{B} [symbol] in the formulation.

Certain embodiments implement Wigner-Eckart to spherical atomic embeddings. Since O_{AA} is only locally ‘seen’ by atom A without geometric constraints from surrounding atoms, some embodiments extract features that do not depend on the orientation of |Φ_{A} [symbol] without loss of information. A correspondence utilizing the Wigner-Eckart theorem:

[symbol]

Yₗ_{₁}ₘ_{₁}|Tₘ^{(l)}|Yₗ_{₂}ₘ_{₂} [symbol] =Tₗ_{₁}_{,l}_{₂}^{(l)}Cₗ_{₂}ₘ_{₂}_{;lm}^{l}^{₁}^{,m}^{₁} (5)

where \hat{T}ₘ^{(l)} is an irreducible spherical tensor operator of rank l and degree m, defined as [symbol] \hat{r}|\hat{T}ₘ^{(l)}|\hat{r}′ [symbol] =Yₗₘ(\hat{r})δ(\hat{r}−\hat{r}′) where \hat{r}, \hat{r}′∈S²; Tₗ_{₁}_{,l}_{₂}^{(l)} denotes a rotational-invariant scalar value that does not depend on m₁, m₂ and m. Cₗ_{₂}ₘ_{₂}_{;lm}^{l}^{₁}^{,m}^{₁} are Clebsch-Gordan (CG) coefficients known for relating tensor products of representations in SO(3) to its irreducible representations. {\hat{T}ₘ^{(l)}} forms an orthogonal basis for an arbitrary spherical tensor operator \hat{Γ}:L²(S²)→L²(S²), giving rise to the expansion \hat{Γ}=Σ_{1,m}γ^{lm}\hat{T}ₘ^{(l)}. In the absence of indices A and n, given [symbol] Yₗ_{₁}ₘ_{₁}|\hat{Γ}|Yₗ_{₂}ₘ_{₂} [symbol] , γ^{lm} can be recovered as the desired basis-independent embeddings. Keeping this motivation, for [symbol] Φ_{A}| [symbol] |Φ_{A} [symbol] , atomic embeddings h_{A}^{n,l,m} in accordance with certain embodiments can be obtained by making use of a set of auxiliary basis {|\tilde{Φ}_{A}^{n,l,m} [symbol] }:

$${h}_{A}^{n , l , m , p = 0} = {\sum }_{{n}_{1} , {n}_{2}} {\sum }_{{l}_{1} , {l}_{2}} {\sum }_{{m}_{1} , {m}_{2}} 〈 {Φ}_{A}^{{n}_{1} , {l}_{1} , {m}_{1}} \vert \hat{𝒪} \vert {Φ}_{A}^{{n}_{2} , {l}_{2} , {m}_{2}} 〉 ( {\overline{\tilde{Φ}}}_{A}^{n , l , m} \vert ( \vert {Φ}_{A}^{{n}_{1} , {l}_{1} , {m}_{1}} 〉 ⊗ \vert {Φ}_{A}^{{n}_{2} , {l}_{2} , {m}_{2}} 〉 ) \qquad ( 6 )$$

where |Φ_{A}^{n,l,m} [symbol] are constructed as products of Gaussian functions and spherical harmonics and the basis overlap coefficients [symbol] \tilde{Φ}_{A}^{n,l,m}|(|Φ_{A}ⁿ^{₁}^{,l}^{₁}^{,m}^{₁} [symbol] ⊗|Φ_{A}ⁿ^{₂}^{,l}^{₂}^{,m}^{₂} [symbol] ) can be factorized into scalar contants and CG coefficients; using Eq. 5 and identity relations of CG coefficients, it can be shown that h_{A} at each (n, l) is a spherical tensor covariantly transformed as [symbol] [h_{A}^{n,l}]=D^{l}( [symbol] )·h_{A}^{n,l} under rotation. p∈{0,1} is an index for tracking the parity of a spherical tensor; under inversion [symbol] , a tensor with even (p+l) is invariant, but a tensor with odd (p+l) flips its sign.

Some embodiments provide covariant atomic orbital linear combinations. The non-local blocks O_{AB} encodes interactions between atomic orbitals centered on nuclei positions R_{A} and R_{B}. Since atomic orbitals |Φ_{A} [symbol] and |Φ_{B} [symbol] are spatially separated, O_{AB} cannot be feasibly decomposed into simpler components as done for O_{AA}. Certain embodiments provide a physically-motivated scheme to learn on O_{AB} based on tensor contractions. To perform an update to the attribute on atom centers h_{A}^{t} [symbol] h_{A}^{t+1}, a set of gauge tensor V_{AB}^{t,i} in accordance with some embodiments can be learned for each pair of atoms (A, B):

(V_{AB}^{t,i})^{l,m}=W_{1,i}^{l,t}(h_{A}^{t})^{l,m,p=0}+Yₗₘ(\hat{R}_{AB})(W_{2,i}^{l,t}∥h_{A}^{t}∥) (7)

where \hat{R}_{AB} is the Cartesian direction vector between atom centers A and B, ∥•∥ denotes taking the gauge-invariant content of a spherical tensor, ∥x∥=⊕_{l,p}\sqrt{Σₘ₌₋ₗ^{l}(x^{l,m,p})²}. Several embodiments provide that W_{1,i}^{l,t} and W_{2,i}^{l,t} are learnable linear functions. Eq. 7 is a linear map on spherical tensors h_{A}^{t} and Y(\hat{R}_{AB}), then it follows that V_{AB}^{t,i} is also a spherical tensor covariant under the actions of O(3). Since the inner product of two spherical tensors of the same rank is an O(3)-invariant scalar, contracting V_{AB}^{t,i} with the bra-dimension of O_{AB} formed by the combined indices (n_{A},l_{A},m_{A}) gives rise to a new spherical tensor defined in its ket-space, the message tensor m_{AB}^{t,i} in accordance with several embodiments:

m_{AB}^{t,i}=V_{AB}^{t,i}·O_{AB}⊕V_{BA}^{t,i}·O_{BA} (8)

Applying the above learnable operations in Eq. 7 and Eq. 8 is equivalent to a linear projection in the Hilbert space spanned by atomic orbitals:

|ψ_{AB}ⁱ [symbol] =V_{AB}^{t,i}|Φ_{A} [symbol] +V_{AB}^{t,i}|Φ_{B} [symbol] ;m_{AB}^{t,i}= [symbol] Ψ_{A}⊕Ψ_{B}|\hat{P} [symbol] |ψ_{AB}ⁱ [symbol] (9)

where |ψ_{AB}ⁱ [symbol] is a linear-combination of atomic orbitals (LCAO), \hat{P}=Î−Σ_{A} O_{AA}|Φ_{A} [symbol] [symbol] (Φ_{A}| is a projector removing the contribution from self-interactions which are already captured by Eq. 6. Consequently m_{AB}^{t,i} is the expectation value of the quantum operator \hat{P} [symbol] in the mixed basis of atomic orbitals (bra-side) and LCAOs (ket-side). Eq. 7 is referred as an LCAO layer.

A number of embodiments provide message passing for AO-LCAO interactions. m_{AB}^{t,i} can be aggregated for updating the representation on atom center A, h_{A}^{t}, analogous to a message passing between nodes and edges in realizations of graph neural networks. Some embodiments incorporate the classical geometric information of atomic positions R through spherical harmonics and couple it with m_{AB}^{t,i}, given by the following proposed O(3)-covariant message passing scheme:

$${( {\tilde{m}}_{A}^{t} )}^{l , m} = {\sum }_{B \neq A} {⊕}_{i , j} ( {( {m}_{A B}^{t , i} )}^{l , m} + {Y}_{l m} ( {\hat{R}}_{A B} ) ( {W}_{3 , i}^{l , t} ‖ {m}_{A B}^{t , i} ‖ ) ) \cdot {a}_{A B}^{t , j} \qquad ( 10 )$$

where W_{3,i}^{l,t} are learnable linear functions, a_{AB}^{t,j} are scalar-valued weights to improve the network capacity which is parameterized as multi-head attentions:

a_{AB}^{t}=MLP((W₄^{t},z_{AB}^{t})⊙κ_{AB}/\sqrt{nₐ}) where z_{AB}^{t}=⊕_{n,l,p}Σₘ₌₋ₗ^{l}(h_{A}^{t})^{n,l,m,p}·(h_{B}^{t})^{n,l,m,p} (11)

κ_{AB}:=W_{κ}(⊕ₖΣₙ_{_{A}}_{,l}_{_{A}}Σₙ_{_{B}}_{,l}_{_{B}}ξ^{k}(∥O_{AB}ⁿ^{_{A}}^{,l}^{_{A}}^{;n}^{_{B}}^{,l}^{_{B}}∥)) are scalar weights shared across all update steps t, where ξ^{k} are Morlet wavelet radial basis functions. MLP denotes a 2-layer multi-layer perceptron, W₄^{t}, W_{κ} are learnable linear functions and nₐ denotes the number of attention heads (i.e. length of a_{AB}^{t}). The attention mechanism Eq. 11 lifts the channel width limitation without increasing memory costs as opposed to explicitly expanding O_{AB}, and it coincides with attentions in SE(3)-transformers. Many embodiments provide that the aggregated equivariant messages \tilde{m}_{A}^{t} are to be interacted with h_{A}^{t} through an equivariant interaction block to complete the update h_{A}^{t} [symbol] h_{A}^{t+1}.

### A Generalized Equivariant Nonlinearity

Many embodiments provide normalization schemes to alleviate the expressive power problem in equivariant neural networks. Equivariant neural networks may be successful at addressing symmetry priors, but many realizations exhibit limitations on nonlinearities. Directly applying activation functions such as ReLU on a spherical tensor (e.g. on xyz components of a vector) may violate equivariance. This issue is also present in point-cloud-based equivariant molecular neural networks and can be relieved in some architectures by applying gated operations parameterized by scalar features to l>0 features. However, such approaches may not be integrated with techniques such as Batch Normalization that are known to improve learning, and may pose challenges on setting up the neural network training in practice, such as sensitivity to weight initializations.

Several embodiments implement the normalization scheme including (but not limited to) RepNorm on spherical tensors to alleviate the expressive power problem. Given a spherical tensor x, RepNorm can be defined: x [symbol] (x̅,\hat{x}) where x̅ and \hat{x} are given by:

$${\overline{x}}^{n , l , p} : = \frac{‖ {x}^{n , l , p} ‖ - {μ}_{x}^{n l , p}}{{σ}_{x}^{n , l , p}} and {\hat{x}}^{n , l , m , p} = \frac{{x}^{n , l , m p}}{‖ {x}^{n , l , p} ‖ + 1 / {β}^{n , l , p + ∈}} \qquad ( 12 )$$

where μₓ^{n,l,p} and σₓ^{n,l,p} are mean and variance estimates of the invariant content ∥x∥ which can be obtained from either batch or layer statistics; β^{n,l,p} are positive, learnable scalars controlling the fraction of tensor scale information from x to be retained in \hat{x}, and ϵ is a numerical stability factor set to 10⁻³ in implementation in accordance with certain embodiments. The RepNorm operation in Eq. 12 factorizes the spherical tensor x to the normalized scalar-valued tensor x̅ that allows for being transformed by a scalar NN, and a ‘pure-gauge’ tensor \hat{x} that can be recomposed later to finish updating x. In Eq. 12, 0 is always a fixed point of the map x [symbol] \hat{x} and the direction information in x is not explicitly touched; therefore RepNorm can preserve equivariance and does not introduce artifacts such as unphysical symmetry breaking. RepNorm in accordance with some embodiments improves training stability and eliminates the need for hand-tuning weight initializations and learning rates across different tasks.

### Atomic-Orbital-Based Feature Generation

Many embodiments implement features from a low-cost electronic-structure calculation in the basis of AOs. Some embodiments include a variety of processes that can be utilized to generate AO-based features. AO-based features in OrbNet processes in accordance with several embodiments can be determined by mean field methods. In certain embodiments, AO-based features in OrbNet processes can be computed using (but not limited to) Hartree-Fock theory, density functional theory, or semi-empirical theory. A number of central objects of these methods include (but are not limited to) the Fock (F) matrix, the density (P) matrix, and the overlap (S) matrix. These matrices in accordance with certain embodiments can be determined from the molecular geometry by performing the mean-field computation. Several embodiments implement the matrices to determine the AO-based input features for OrbNet processes.

Many embodiments provide an end-to-end framework to generate AO-based features for OrbNet processes. In some embodiments, the Fock matrix can be parameterized by a neural network including (but not limited to) a graph neural network (GNN). Such embodiments avoid using mean-field computations. Certain embodiments provide that the Fock matrix parameterization:

F=Dec[GNN(R,Z)] (13)

Where R are the nuclear coordinates of the atoms in the molecule, Z are the atomic numbers of the atoms in the molecule, and Dec is a decoding module. Several embodiments provide that the nodes of GNN correspond to atoms and the edges correspond to interatomic interactions. The elements of the Fock matrix are:

F_{μv}=Dec_{l(μ),l(v)}^{e}[h(μ)[GNN(R,Z)],h(v)[GNN(R,Z)]]S_{μv} (14)

where μ and v index AO basis functions and l(μ) is the total angular momentum corresponding to basis function l, and h(μ)[GNN( . . . )] is the node representation corresponding to the atom on which basis function μ is centered.

The form of the decoder Dec_{l(μ),l(v)}^{e}, is a multilayer perceptron (MLP) in accordance with some embodiments. It is indexed by a pair of AO angular momenta. In several embodiments, it may be implemented as a set of MLPs with one MLP per angular momentum pair. In certain embodiments, it may be implemented as a single multi-task MLP whose heads each correspond to an angular momentum. Several embodiments represent quantum mechanical matrices in the STO-6G basis set. Many embodiments provide that the GNN can be trained either separately from the OrbNet model or in combination with the OrbNet model.

Many embodiments provide that OrbNet features can be determined from the Fock matrix. In some embodiments, the density matrix may be determined by diagonalizing the Fock matrix:

$$\begin{matrix}FC = SC ∈ & ( 15 ) \\ {P}_{μ v} = {\sum }_{i = 1}^{{n}_{elec} / 2} {C}_{μ i}^{*} {C}_{vi} & ( 16 )\end{matrix}$$

where n_{elec}/2 is the number of electrons in the molecule and * denotes complex conjugation.

As can readily be appreciated, any of a variety of operations can be evaluated for the AOs which can be used as input AO-based features and any of a variety of input AO-based features can be selected as appropriate to the requirements of a specific application.

### OrbNet for Atomic-Orbital-Based Features

Many embodiments provide equivariant interaction block as a modular component to construct [symbol] _{θ}, performing an update h_{A}^{t} [symbol] h_{A}^{t+1} given another spherical tensor g_{A} (e.g. \tilde{m}_{A}^{t} in Eq. 10, or h_{A}^{t} itself) to be interacted with h_{A}^{t}:

f_{A}^{t}=MLP₁(h̅_{A}^{t})⊙ĥ_{A}^{t}, where (h̅_{A}^{t},ĥ_{A}^{t})=RepNorm(h_{A}^{t}) (17)

u_{A}^{n,l,m,p}=g_{A}^{n,l,m,p}+Σₗ_{₁}_{,l}_{₂}Σₘ_{₁}_{,m}_{₂}Σₚ_{₁}_{,p}_{₂}(f_{A}^{t})^{n,l}^{₁}^{,m}^{₁}^{,p}^{₁}g_{A}^{n,l}^{₂}^{,m}^{₂}^{,p}^{₂}Cₗ_{₁}ₘ_{₁}_{;l}_{₂}ₘ_{₂}^{lm}δₗ_{₁}₊ₗ_{₂}₋ₗ^{p}^{₁}^{+p}^{₂}^{-p} (18)

h_{A}^{t+1}=h_{A}^{t}+MLP₂(û_{A})⊙û_{A}, where(ū_{A},û_{A})=RepNorm(u_{A}) (19)

where δᵢ^{j} is a Kronecker delta function, and MLP₁ and MLP₂ denote multi-layer perceptrons. For computational efficiency, in the parity-aware spherical tensor coupling in Eq. 18, the angular momentum indices (l₁,l₂) in accordance with some embodiments are restricted within the range {(l₁,l₂); l₁+l₂<lₘₐₓ,
where lₘₐₓ is the maximum angular momentum considered in the implementation.

Once the representations h_{A}^{t} are updated to the last step h_{A}^{t}^{_{f}}, a pooling operation {h_{A}^{t}^{_{f}}} [symbol] y_{θ} in accordance with several embodiments can be employed to readout the target prediction y∈ [symbol] . Due to gauge-equivariance in the OrbNet model formulation, the physics prior of the learning task can be flexibly addressed through designing pooling schemes without the need of modifying the model architecture. Pooling operations can be defined for representative classes of quantum-chemical properties based on the algebra structure of [symbol] and whether y is an extensive or intensive property. This setting enables learning on challenging tensorial properties including (but not limited to) dipole moments and electron density.

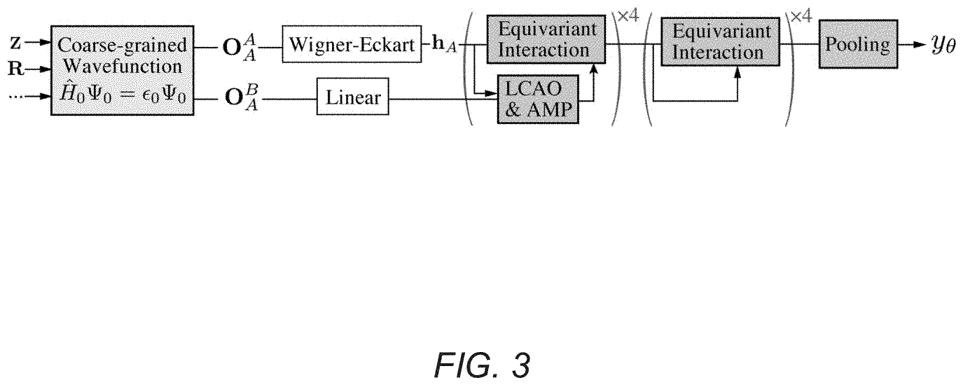

Some embodiments assemble OrbNet models, namely [symbol] _{θ}, by stacking the NN building blocks. A top-level view of the architecture of OrbNet for AO based features in accordance with an embodiment of the invention is illustrated in FIG. 3.

### Symmetry-Adapted-Atomic-Orbital-Based Feature Generation

Many embodiments implement features from a low-cost electronic-structure calculation in the basis of SAAOs. Many embodiments include a variety of processes that can be utilized to generate SAAOs features. In several embodiments, SAAOs can be derived from the set and/or a subset of transformed atomic orbital basis for the molecular system and/or other external potential. Certain embodiments provide that the SAAOs can be obtained via the reduced density matrix of the molecular system in the atomic orbital representation. In a number of embodiments, the SAAOs can be obtained via schemes based on eigenvalues of the Fock matrix in the atom orbital representation and/or the Wigner rotations. Several embodiments provide that SAAO features can be scalar and/or tensor quantities derived from expectation values of quantum operators and/or the derivatives of expectation values of quantum operators with respect to the SAAOs. Examples of quantum operators include (but are not limited to): elements of a Fock (F) matrix, elements of a Coulomb (J) matrix, elements of a Hartree-Fock exchange (K) matrix, elements of a density (P) matrix, element of an orbital centroid distance (D) matrix, element of a core Hamiltonian (H) matrix, and/or element of an overlap (S) matrix. Some embodiments implement SAAO features based on quantum operators in (tight-binding) density functional theory calculations and/or other semi-empirical electronic structure theory methods including (but not limited to): the shell-resolved charges and approximations to the J, K, F, P, D, H, S, and/or exchange-correlation operators. Many embodiments provide the operators can be in Kohn-Sham density functional theory including (but not limited to): the exchange-correlation operator, the exchange-correlation operators' approximations, and the exchange-correlation operators' components. Several embodiments include that quantum operators can be properties of the molecular systems. Examples of the properties include (but are not limited to): dipole moment, interatomic distance matrix, continuum solvation energy. As can readily be appreciated, any of a variety of quantum operations can be evaluated for the AO which can be used as input SAAOs features and any of a variety of input SAAOs features can be selected as appropriate to the requirements of a specific application.

Sets of SAAO features in many embodiments have no explicit dependence on atom types, thus OrbNet processes can enhance chemical transferability of the training results. In several embodiments, the smooth variation and local linearity of pair correlation energies as a function of SAAO features of different molecular geometries and different molecules can be beneficial to the transferability of OrbNet processes.

Many embodiments implement a transferable mapping from input feature values {f} to the regression labels that are quantum mechanical energies,

E≈E^{ML}[{f}] (20)

Several embodiments provide the generation of SAAO features. Let {Φ_{n,l,m}^{A}} be the set of AO basis functions with atom index A and the standard principal and angular momentum quantum numbers, n, l, and m. Let C be the corresponding molecular orbital coefficient matrix obtained from a mean-field electronic structure calculation, such as HF theory, DFT, or a semi-empirical method. The one-electron density matrix of the molecular system in the AO basis is then

$${P}_{µ v} = 2 {\sum }_{i ∈ o c c} {C}_{µ i} {C}_{v i} \qquad ( 21 )$$

(for a closed-shell system). A rotationally invariant symmetry-adapted atomic-orbital (SAAO) basis {Φ_{n,l,m}^{A}} by diagonalizing diagonal density-matrix blocks associated with indices A, n, and l, can be constructed, such that

Pₙₗ^{A}Yₙₗ^{A}=Yₙₗ^{A}diag(λₙₗₘ^{A}) (22)

where [Pₙₗ^{A}]_{mm′}=P_{nlm,nlm′}^{A}. For s orbitals (l=0), this symmetrization procedure can be trivial, and can be skipped. By construction, SAAOs are localized and consistent with respect to geometric perturbations of the molecule, and in contrast with localized molecular orbitals (LMOs) obtained from minimizing a localization objective function (Pipek-Mezey, Boys, etc.), SAAOs can be obtained by a series of very small diagonalizations, without the need for an iterative procedure. The SAAO eigenvectors Yₙₗ^{A} are aggregated to form a block-diagonal transformation matrix Y that specifies the full transformation from AOs to SAAOs:

$$\vert {\hat{Φ}}_{p} 〉 = {\sum }_{µ} {Y}_{µ p} \vert {Φ}_{μ} \qquad ( 23 )$$

where μ and p index the AOs and SAAOs respectively.

Several embodiments employ ML features {f} comprised of tensors obtained by evaluating quantum-chemical operators in the SAAO basis. Hereafter, all quantum mechanical matrices can be represented in the SAAO basis, including the Fock matrix (F), the Coulomb matrix (J), and the Hartree-Fock exchange matrix (K), the density matrix (P), orbital centroid distance matrix (D), the core Hamiltonian matrix (H), and the overlap matrix (S).

Many embodiments provide approximated Coulomb and exchange SAAO feature generation. When a semi-empirical quantum chemical theory is employed, the computational bottleneck of SAAO feature generation becomes the J and K terms, due to the need to compute four-index electron-repulsion integrals. Some embodiments implement a generalized form of the Mataga-Nishimoto-Ohno-Klopman formula, as in the sTDA-xTB method,

$${( pq \vert rs )}^{MNOK} = {\sum }_{A} {\sum }_{B} {Q}_{oq}^{A} {Q}_{rs}^{B} {γ}_{AB} \qquad ( 24 )$$

Here, A and B are atom indices, p, q, r, s are SAAO indices, and

$${γ}_{AB}^{\{ J , K ]} = {( \frac{1}{{R}_{AB}^{{y}_{\{ J , K \}}} + {η}^{- {y}_{\{ J , K \}}}} )}^{1 / {y}_{\{ J , K \}}} \qquad ( 25 )$$

where R_{AB} is the distance between atoms A and B, η is the average chemical hardness for the atoms A and B, and y_{{J,K}} are empirical parameters specifying the decay behavior of the damped interaction kernels, γ_{AB}^{{J,K}}. In certain embodiments, y_{{J}}=4 and y_{{K}}=10 are used. The transition density Q_{pq}^{A} is calculated from a Löwdin population analysis,

$${Q}_{pq}^{A} = {\sum }_{µ ∈ A} {Y}_{µ p}^{′} {Y}_{µ p}^{′} \qquad ( 26 )$$

where the pth column of Y′=YS^{1/2} contains the expansion coefficients for the pth SAAO in the symmetrically orthogonalized AO basis. This yields approximated J and K matrices for featurization,

$$\begin{matrix}{J}_{pq}^{MNOK} = {( pp \vert qq )}^{MNOK} = {\sum }_{A , B} {Q}_{pp}^{A} {Q}_{qq}^{B} {γ}_{AB}^{I} & ( 27 ) \\ {K}_{pq}^{MNOK} = {( pp \vert qq )}^{MNOK} = {\sum }_{A , B} {Q}_{pp}^{A} {Q}_{qq}^{B} {γ}_{AB}^{K} & ( 28 )\end{matrix}$$

A naive implementation of Eqs.27 and 28 is [symbol] (N⁴), the leading asymptotic cost. However, this scaling may be reduced to [symbol] (N²) with negligible loss of accuracy through a tight-binding approximation. Computation of J^{MNOK} and K^{MNOK} is not the leading order cost for feature generation and such tight-binding approximation is thus not employed.

While various processes for generating SAAOs features for OrbNet processes are described above, any of a variety of processes that can generate SAAOs features can be utilized in the OrbNet processes as appropriate to the requirements of specific applications in accordance with various embodiments of the invention. Processes for designing graph neural network models for OrbNet processes with SAAOs features in accordance with various embodiments of the invention are discussed further below.

### OrbNet for Symmetry-Adapted-Atomic-Orbital-Based Features

In many embodiments, OrbNet processes provide efficient evaluation of the features in the SAAO basis. A number of embodiments of the invention utilize machine learning models including (but not limited to) Graph Neural Network (GNN) models that receive SAAO features as a direct input and output estimates of molecular properties for the received SAAO features as an output. Several embodiments provide that OrbNet utilizes a GNN architecture with edge and node attention and message passing layers, and a prediction phase to ensure extensivity of the resulting energies. Many embodiments provide the mapping of features from semi-empirical-quality features to DFT-quality labels with OrbNet processes. Certain embodiments provide that OrbNet processes can be implemented in the mean-field method used for features (i.e. allowing for Hartree-Fock, DFT, etc.) and the level of theory used for generating labels (i.e. allowing for coupled-cluster and other correlated-wavefunction-method reference data). Various ways in which OrbNet processes can estimate molecular properties from sets of features describing molecular systems in accordance with different embodiments of the invention are discussed further below.

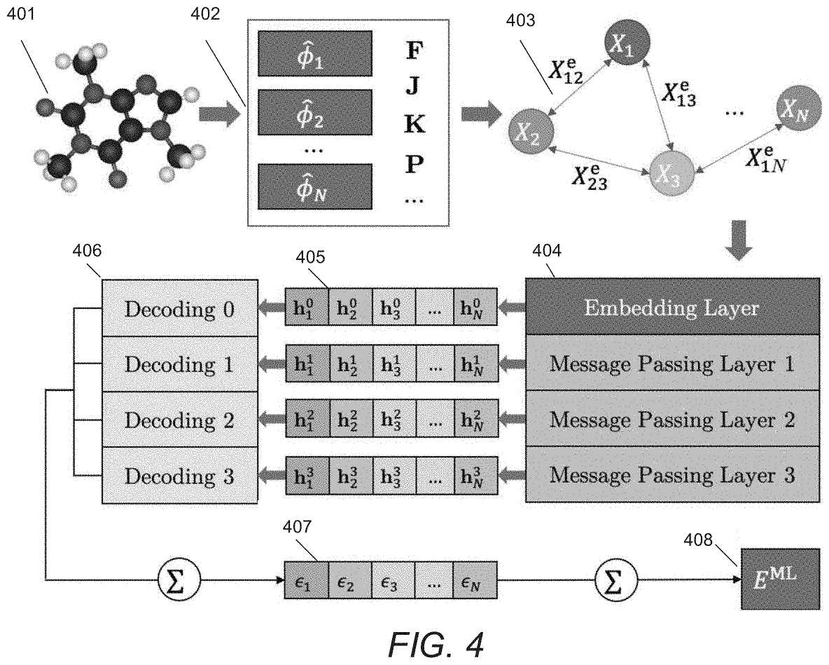

Many embodiments implement OrbNet for SAAO features to encode the molecular system as graph-structured data and utilize a graph neural network (GNN) machine-learning architecture. The GNN represents data as an attributed graph G (V, E, X, X^{e}), with nodes V, edges E, node attributes X: V→ [symbol] ^{n×d}, and edge attributes X^{e}: E→ [symbol] ⁿ^{ₑ}^{×e}, where n=|V|, nₑ=|E|, and d and e are the number of attributes per node and edge, respectively. A graph depicting the workflow of the OrbNet process in accordance with an embodiment of the invention is illustrated in FIG. 4. A low-cost mean-field electronic structure calculation can be performed for the molecular system (401). The resulting SAAOs and the associated quantum operators can be constructed (402). An attributed graph representation (403) can be built with node and edge attributes corresponding to the diagonal and off-diagonal elements of the SAAO tensors. The attributed graph can be processed by the embedding layer and message passing layers (404) to produce transformed node and edge attributes. The transformed node attributes for the encoding layer and each message passing layer can be extracted (405) and passed to MPL-specific decoding networks (406). The node-resolved energy contributions ϵᵤ can be obtained by summing the decoding networks outputs node-wise (407), and the final extensive energy prediction (408) can be obtained from a one-body summation over the nodes.

In several embodiments, OrbNet employs a graph representation for a molecular system in which node attributes correspond to diagonal SAAO features Xᵤ=[Fᵤᵤ,Jᵤᵤ,Kᵤᵤ,Pᵤᵤ,Hᵤᵤ] and edge attributes correspond to off-diagonal SAAO features Xᵤᵥ^{e}=[Fᵤᵥ,Jᵤᵥ,Kᵤᵥ,Dᵤᵥ,Pᵤᵥ,Sᵤᵥ,Hᵤᵥ]. By introducing an edge attribute cutoff value for edges to be included, non-interacting molecular systems separated at infinite distance are encoded as disconnected graphs, thereby satisfying size-consistency.

The model capacity can be enhanced by introducing nonlinear input-feature transformations to the graph representation via radial basis functions,

hᵤ^{RBF}=[Φ₁^{h}(\tilde{X}ᵤ),Φ₂^{h}(\tilde{X}ᵤ), . . . ,Φₙ_{ᵣ}^{h}(\tilde{X}ᵤ)] (29)

eᵤᵥ^{RBF}=[Φ₁^{e}(\tilde{X}ᵥᵤ^{e}),Φ₂^{e}(\tilde{X}ᵤᵥ^{e}), . . . ,Φₘ_{ᵣ}^{e}(\tilde{X}ᵤᵥ^{e})] (30)

where \tilde{X} and \tilde{X}^{e} are n×d and m×e matrices with pre-normalized attributes. Sine basis functions Φₙ^{h}(r)=sin(Σnr) are used for node embedding. Some embodiments employ 0-th order spherical Bessel functions for edge embedding,

$${Φ}_{m}^{e} ( r ) = {j}_{0}^{m} ( r / {c}_{X} ) \cdot {I}_{X} ( r ) = \sqrt{\frac{2}{{c}_{X}}} \frac{sin ( π mr / {c}_{X} )}{r / {c}_{X}} \cdot {I}_{X} ( r ) \qquad ( 31 )$$

where c_{X} (X∈{F, J, K, D, P, S, H}) is the operator-specific upper cutoff value to \tilde{X}ᵤᵥ^{e}. To ensure that the feature varies smoothly when a node enters the cutoff, some embodiments implement the mollifier I_{X}(r):

$${I}_{X} ( r ) = \{ \begin{matrix}exp ( - \frac{{c}_{X}^{2}}{{( \vert r \vert - {c}_{X} )}^{2}} + 1 ) , & if 0 \leq \vert r \vert < {c}_{X} \\ 0 , & if \vert r \vert \geq {c}_{X}\end{matrix} \qquad ( 32 )$$

Note that Φₘ^{e}(r) decays to zero as an edge approaches the cutoff to ensure size-consistency, and the mollifier is infinite order differentiable at the boundaries, which eliminates representation noise that can arise from geometric perturbation of the molecule. To enforce that the output is constant at machine precision when adding arbitrary numbers of zero edge features, which is critical for the extraction of analytical gradients and training potential energy surfaces, some embodiments implement an ‘auxiliary edge’ scheme to be integrated with the message passing mechanism,

eᵤᵥ^{aux}=W^{aux}·eᵤᵥ^{RBF} (33)

where W^{aux} is a trainable parameter matrix. The radial basis function embeddings are transformed by neural network modules to yield 0-th order node and edge attributes,

hᵤ⁰=Encₕ(eᵤᵥ^{RBF}), eᵤᵥ⁰=Encₑ(hᵤ^{RBF}) (34)

where Encₕ and Encₑ are residual blocks comprising 3 dense neural network layers. In contrast to atom-based message passing neural networks in accordance with some embodiments, this additional embedding transformation captures the interactions among the physical operators.

The node and edge attributes are updated via the Transformer-motivated message passing mechanism. For a given message passing layer (MPL) t+1, the information carried by each edge can be encoded into a message function mᵤᵥ^{t} and associated attention weight mᵤᵥ^{t}, and can be accumulated into node features through a graph convolution operation. The overall message passing mechanism is given by:

$${h}_{u}^{t + 1} = {h}_{u}^{t} + σ ( {W}_{h}^{t} \cdot [ {⊕}_{j} ( {\sum }_{v ∈ N ( u )} {w}_{uv}^{t , j} \cdot {m}_{uv}^{t} ) ] + {b}_{h}^{t} ) \qquad ( 35 )$$

where mᵤᵥ^{t} is the message function computed on each edge:

mᵤᵥ^{t}=σ(Wₘ^{t}·[hᵤ^{t}⊙hᵥ^{t}⊙eᵤᵥ^{t}]+bₘ^{t}) (36)

and the convolution kernel weights, wᵤᵥ^{t,j}, are evaluated as (multi-head) attention scores to characterize the relative importance of an orbital pair,

wᵤᵥ^{t,j}=σₐ(Σ[(Wₐ^{t,j}·hᵤ^{t})⊙(Wₐ^{t,j}·hᵥ^{t})⊙eᵤᵥ^{t}⊙eᵤᵥ^{aux}]/nₑ) (37)

where the summation is applied over the elements of the vector in the summand. Here, the index j specifies a single attention head, and nₑ is the dimension of hidden edge features eᵤᵥ^{t}, ⊕ denotes a vector concatenation operation, ⊙ denotes the Hadamard product, and · denotes the matrix-vector product. The edge attributes can be updated according to

eᵤᵥ^{t+1}=σ(Wₑ^{t}·mᵤᵥ^{t}+bₑ^{t}) (38)

Wₘ^{t}, Wₕ^{t}, Wₑ^{t}, bₘ^{t}, bₕ^{t}, bₑ^{t} are MPL-specific trainable parameter matrices, Wₐ^{t,i} are MPL- and attention-head-specific trainable parameter matrices, σ(·) is an activation function with a normalization layer, and σₐ(·) is the activation function used for generating attention scores.

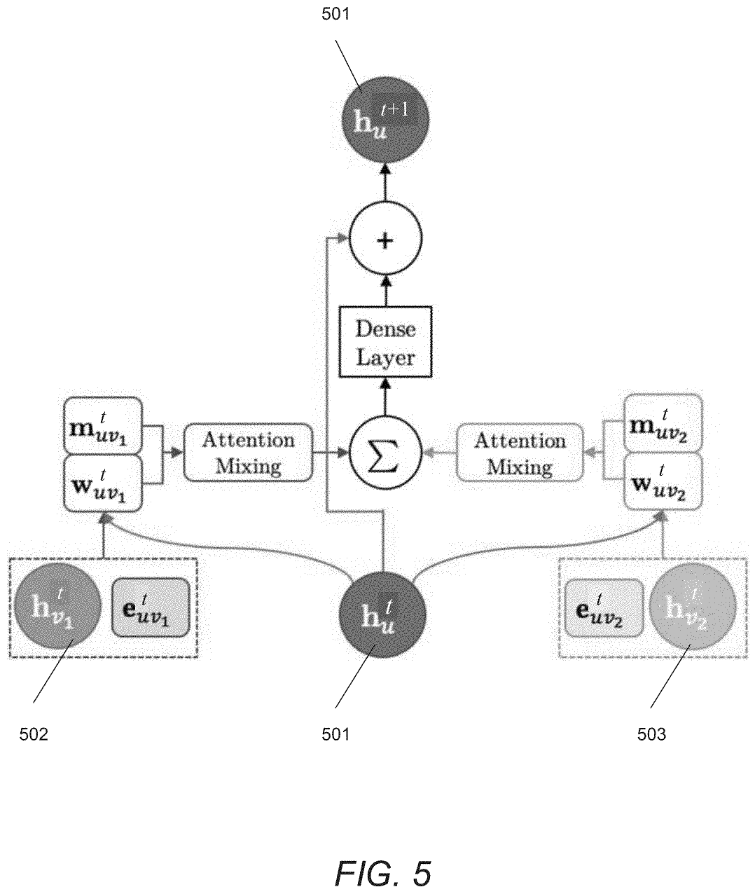

A graph of the OrbNet message passing layer (MPL) for SAAOs in accordance with an embodiment of the invention is illustrated in FIG. 5. For the t+1 MPL, the attributes of a given node (501) can be updated due to interactions with nearest-neighbor nodes (502 and 503), which depend on both the nearest-neighbor node attributes and the nearest-neighbor edge attributes. The node and edge features (i.e., hᵤ^{t}, hᵥ^{t}, and eᵤᵥ^{t}) combine to produce a message mᵤᵥ^{t} (Eq. 32) and multi-head attention score wᵤᵥ^{t} (Eq. 33) which undergo attention mixing. The attention-weighted messages from each nearest-neighbor node and edge are combined and passed into a dense layer, the result of which is added to the original node attributes to perform the update (Eq. 31).

The decoding phase of OrbNet in accordance with several embodiments can be designed to ensure the size-extensivity of energy predictions. The employed mechanism outputs node-resolved energy contributions for the embedding layer (t=0) and all MPLs (t=1, 2, . . . , T) to predict the energy components associated with all nodes and MPLs. The final energy prediction E^{ML} can be obtained by first summing over l for each node u and then performing a one-body sum over nodes (i.e., orbitals), such that

$${E}^{ML} = {\sum }_{u ∈ V} {ɛ}_{u} = {\sum }_{u ∈ V} {\sum }_{t = 0}^{T} {Dec}^{t} ( {h}_{u}^{t} ) \qquad ( 39 )$$

where the decoding networks Dec^{t} are multilayer perceptrons.

Many embodiments incorporate a multi-task learning strategy in OrbNet processes to improve learning efficiency. In several embodiments, OrbNet processes can be trained with both molecular energies and other computed properties of the quantum mechanical wavefunction. To enable multi-task learning and to improve the learning capacity of the OrbNet model, several embodiments implement atom-specific attributes, f_{A}^{t}, and global molecule-level attributes, q^{t}, where t is the message passing layer index and A is the atom index. The whole-molecule and atom-specific attributes allow for the prediction of auxiliary targets through multi-task learning, thereby providing physically motivated constraints on the electronic structure of the molecule that can be used to refine the representation at the level of AO-based features.

Several embodiments provide the analytical gradient theory for OrbNet. The analytical gradient theory for OrbNet in accordance with certain embodiments may be essential for the calculation of inter-atomic forces and other response properties including (but not limited to) dipoles and linear-response excited states.

In many embodiments, for the prediction of both the electronic energies and the auxiliary targets, only the final atom-specific attributes, f_{A}^{T}, are employed, since they self-consistently incorporate the effect of the whole-molecule and node- and edge-specific attributes. The electronic energy can be obtained by combining the approximate energy E_{TB} from the extended tight-binding calculation and the model output E_{NN}, the latter of which is a one-body sum over atomic contributions; the atom-specific auxiliary targets d_{A} can be predicted from the same attributes.

$$\begin{matrix}{\hat{E}}_{out} = {E}_{TB} + {E}_{NN} = {E}_{TB} + {\sum }_{A} {∈}_{A} = {E}_{TB} + {\sum }_{A} [ Dec ( {f}_{A}^{T} ) + {E}_{A}^{C} ] & ( 40 ) \\ {\hat{d}}_{A} = {Dec}^{aux} ( {f}_{A}^{T} ) & ( 41 )\end{matrix}$$

Here, the energy decoder Dec and the auxiliary-target decoder Dec^{aux} are residual neural networks built with fully connected and normalization layers, and EE are element-specific, constant shift parameters for the isolated-atom contributions to the total energy.

Many embodiments provide that the OrbNet processes can be end-to-end differentiable by employing input features including (but not limited to) the AO-based features that are smooth functions of both atomic coordinates and external fields. Several embodiments provide the analytic gradients of the total energy E^{out} with respect to the atom coordinates. Some embodiments employ local energy minimization with respect to molecular structure to demonstrate the quality of the learned potential energy surface.

Using a Lagrangian formalism, the analytic gradient of the predicted energy with respect to an atom coordinate x can be expressed in terms of contributions from the tight-binding model, the neural network, and additional constraint terms:

$$\frac{d {E}_{o u t}}{d x} = \frac{d {E}_{T B}}{d x} + {\sum }_{f ∈ \{ F , D , P , S , H \}} [ \frac{\partial {E}_{N N}}{\partial f} \frac{\partial f}{\partial x} ] + Tr [ W \frac{\partial {S}^{A O}}{\partial x} ] + T r [ z \frac{\partial {F}^{A O}}{\partial x} ] \qquad ( 42 )$$

Here, the third and fourth terms on the right-hand side are gradient contributions from the orbital orthogonality constraint and the Brillouin condition, respectively, where F^{AO} and S^{AO} are the Fock matrix and orbital overlap matrix in the atomic orbital (AO) basis. In some embodiments, the analytical gradient for OrbNet can be based on a tight-binding (GFN-xTB) model. The tight-binding gradient

$$\frac{d {E}_{T B}}{d x}$$

in accordance with several embodiments can be the tight-binding gradient. In certain embodiments, the neural network gradients with respect to the input features

$$\frac{\partial {E}_{N N}}{\partial f}$$

can be obtained using reverse-mode automatic differentiation.

Several embodiments implement graph- and atom-level auxiliary tasks to improve the generalizability of the learned representations for molecules. Some embodiments employ multi-task learning with respect to the total molecular energy and atom-specific auxiliary targets. The atom-specific targets can be similar to the features introduced in the DeePHF model, obtained by projecting the density matrix into a basis set that does not depend upon the identity of the atomic element,

dₙₗ^{A}=[EigenVals_{m,m′}([^{O}Dₙₗ^{A}]_{m,m′})∥EigenVals_{m,m′}([^{V}Dₙₗ^{A}]_{m,m′})] (43)

Here, the projected density matrix is given by ([^{O}Dₙₗ^{A}]_{m,m′}=Σ_{i∈occ} [symbol] αₙₗₘ^{A}|Ψᵢ [symbol] [symbol] Ψᵢ|α_{nlm′}^{A} [symbol] , and the projected valence-occupied density matrix is given by ([^{V}Dₙₗ^{A}]_{m,m′}=Σj_{∈valocc} [symbol] αₙₗₘ^{A}|Ψⱼ [symbol] [symbol] Ψⱼ|α_{nlm′}^{A} [symbol] , where |Ψ_{i,j} [symbol] are molecular orbitals from the reference DFT calculation, |αₙₗₘ^{A} [symbol] is a basis function centered at atom A with radial index n and spherical-harmonic degree l and order m. The indices i and j runs over all occupied orbitals and valence-occupied orbital indices, respectively, and ∥ denotes a vector concatenation operation. The auxiliary target vector d_{A} for each atom A in the molecule is obtained by concatenating dₙₗ^{A} for all n and l.

While various processes for designing graph neural networks with message passing layers for OrbNet processes for SAAO features are described above with reference to FIG. 4 and FIG. 5, any of a variety of processes that utilize deep learning models can be utilized in the design of OrbNet processes implementing SAAOs features as appropriate to the requirements of specific applications in accordance with various embodiments of the invention. Processes for identifying AO-based feature distance metrics in accordance with various embodiments of the invention are discussed further below.

### Chemical Space Structure Discovery

Processes in accordance with various embodiments of the invention can rely upon the use of distance metrics that measure the distance between the AO-based features including (but not limited to) SAAO features of different molecular systems in feature space. In many embodiments, chemical space structure discovery is further enhanced by utilizing subspace embedding techniques to discover the local and global structures of AO feature space. As is discussed further below, any of a variety of distance measures and/or structure discovery techniques can be utilized as appropriate to the requirements of specific applications in accordance with various embodiments of the invention.

Many embodiments implement AO features including (but not limited to) a set of distance measures between a number of AOs including (but not limited to): a pair, a trio, a quartet, in the space of AO features. In this space, a distance can be defined which distinguishes pairs based on their AO features. As can readily be appreciated, any of a variety of distance metric implementations can be utilized as appropriate to the requirements of specific applications in accordance with various embodiments of the invention.

While systems and methods that include various AO feature distance metrics are described above, any of variety of processes for measuring distance between the AO-based features of different molecular systems can be utilized in OrbNet processes as appropriate to the requirements of specific applications in accordance with various embodiments of the invention. Processes for generating databases of AO-based features in accordance with various embodiments of the invention are discussed further below.

### Generating Databases of AO-Based Features

Processes in accordance with various embodiments of the invention are capable of generating databases of AO-based features. As is discussed further below, any of a variety of AO-based feature databases can be utilized as appropriate to the requirements of specific applications in accordance with various embodiments of the invention.

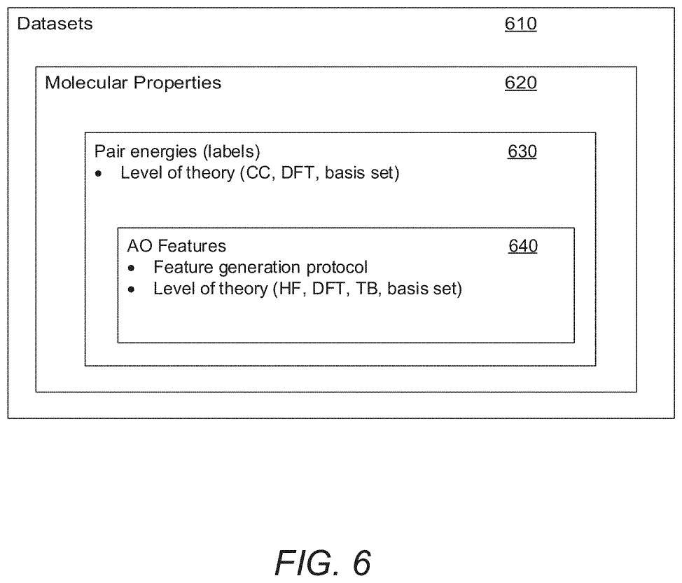

Many embodiments implement OrbNet processes that store, organize, and classify databases that include (but are not limited to) atomic orbitals which form the basis for the feature values associated with an AO basis and/or SAAOs. In some embodiments, the AO-basis- and/or SAAO-associated feature values can be output from OrbNet processes, using processes similar to those described above with respect to FIG. 1. In some embodiments, an AO-based feature database is utilized that is organized based on a set of distance measures between a number including (but not limited to): a pair, a trio, and a quartet, of atomic orbitals in the AO original feature space and/or a subspace and/or latent space of the AO feature space. FIG. 6 schematically illustrates database structures in accordance with an embodiment of the invention. The databases 610 can contain molecular properties 620. The molecular properties can include (but are not limited to) associated pair energies 630. The associated pair energies can be calculated using processes including (but not limited to) coupled cluster theory and/or DFT theory. The associated pair energies can be utilized to determine input AO-based features including (but not limited to) SAAO features 640. The AO-based features can be determined by (but not limited to) feature generation protocols applying various levels of quantum chemistry theories such as semi-empirical tight-binding, different basis sets from Hartree-Fock (HF), or different basis sets from density function theory (DFT). As can readily be appreciated, the specific features used in the generation of AO-based feature databases are largely only limited by the requirements of specific applications. Furthermore, databases can be generated using more complex representations of quantum chemical information including (but not limited to) attributed graphs. In several embodiments, databases are constructed in which quantum chemical information for molecular systems is described using attributed graphs constructed using atomic-orbital-based features G(V, E, X, X^{e}) with node features corresponding to diagonal AO blocks and edge features corresponding to off-diagonal AO blocks. Some embodiments implement node features corresponding to SAAO features (Xᵤ=[Fᵤᵤ,Jᵤᵤ,Kᵤᵤ,Pᵤᵤ,Hᵤᵤ]) and the edge features corresponding to off-diagonal SAAO features (X^{e}ᵤᵥ=[Fᵤᵥ,Jᵤᵥ,Kᵤᵥ,Dᵤᵥ,Pᵤᵥ,Sᵤᵥ,Hᵤᵥ]). In a number of embodiments, quantum chemical information represented as attributed graphs in this way can be utilized within a variety of OrbNet processes including (but not limited to) OrbNet processes that perform multi-task learning to learn associations between the attributed graph structures and chemical properties from a training data set. A benefit of the graph representation is that they can provide permutation invariance and size-extensivity, and be utilized for general chemical property classification or regression utilizing techniques including (but not limited to) a graph neural network incorporating a generalized message-passing mechanism. As can readily be appreciated, quantum chemical information can be represented using any of a variety of techniques and/or structures within databases and the represented information can be utilized in a variety of machine learning and/or generative processes similar to those described herein to facilitate the synthesis of molecular systems having desirable chemical properties as appropriate to the requirements of specific applications. Accordingly, embodiments of the invention should be understood as not being limited to any particular representation of quantum chemical information, but instead by understood as general techniques that are applicable to any representation of quantum chemical information.

The databases 610 can be queried to generate datasets corresponding to particular sets of molecules, molecular geometries, level of theory, or any combination thereof. Various embodiments employ SQL databases such as MySQL or no-SQL databases such as MongoDB distributed across one or more computers. The databases, according to various embodiments, can be queried to find AO-based features nearby to a given set of AO-based features on the basis of a distance metric measured between sets of AO-based features in the space. Several embodiments enable the databases to be queried to find molecular systems on the basis of the AO-based feature values associated with the atomic orbitals associated with those molecular systems. Examples of such embodiments can include (but are not limited to): employing k-d trees in the space of AO-based features. As can readily be appreciated, any of a variety of implementations of database indexes and/or to facilitate searching can be utilized as appropriate to the requirements of specific applications in accordance with various embodiments of the invention.

While various processes for generating orbital pairs databases are described above, any variety of orbital pairs databases of different molecular systems can be utilized in OrbNet processes as appropriate to the requirements of specific applications in accordance with various embodiments of the invention. Processes for harvesting AO features in accordance with various embodiments of the invention are discussed further below.

### Atomic-Orbital-Based Feature Harvesters

Processes in accordance with various embodiments of the invention rely upon harvesting AO-based features including (but not limited to) SAAO features from quantum chemistry calculations. As is discussed further below, any of a variety of AO-based feature harvesters can be utilized as appropriate to the requirements of specific applications in accordance with various embodiments of the invention.

Many embodiments implement OrbNet processes to collect and harvest AO-based feature values from the output of quantum chemistry calculations. Some embodiments of the AO-based feature values including (but not limited to) SAAO feature values collected from the OrbNet processes can include the AO-based feature values based on the distance between a pair/trio/quartet of molecular orbitals to the AO-based feature values that are stored within a database of atomic orbitals. Some other embodiments of the AO-based feature values collected from the OrbNet processes eliminate the AO-based feature values based on the distance between a pair of atomic orbitals to the AO-based feature values that are stored within the databases of atomic orbitals.

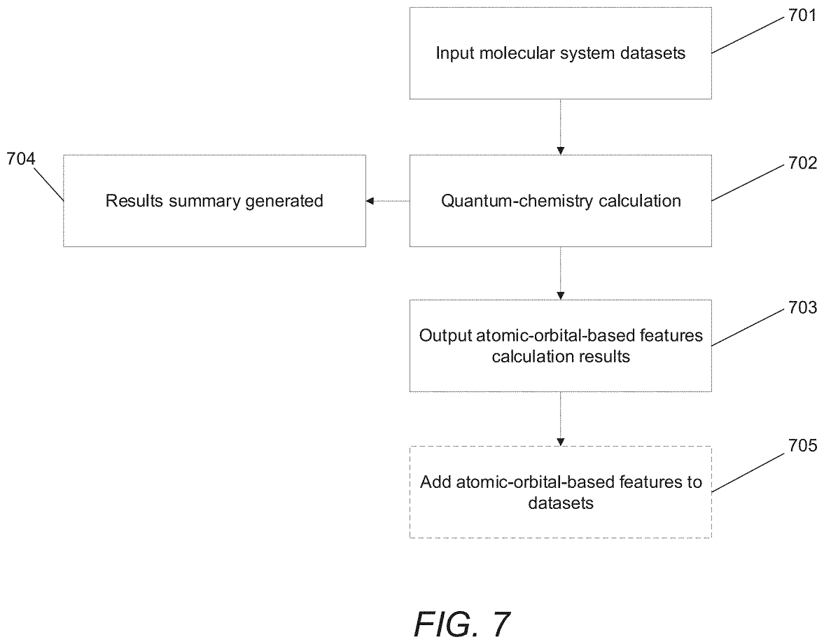

A method for collecting and harvesting AO-based features using an OrbNet process in accordance with an embodiment is illustrated in FIG. 7. Datasets of molecular systems can be generated as input 701. Quantum chemistry calculations can be applied to input datasets 702. Quantum chemistry calculations in accordance with some embodiments can be performed on remote servers including (but not limited to) the internet cloud. The calculation can generate and output corresponding AO-based features 703. These features can be stored in a database of AO-based features 705. Molecules from the calculation results can also be used for synthesis of such molecules 704.

While various processes for harvesting AO features are described above, any variety of processes that can collect and harvest AO features of different molecular systems can be utilized in OrbNet processes as appropriate to the requirements of specific applications in accordance with various embodiments of the invention. Processes for machine learning regression method in accordance with various embodiments of the invention are discussed further below.

### Machine Learning Regression

Processes in accordance with various embodiments of the invention rely upon machine learning techniques including (but not limited to) machine learning regression. As is discussed further below, any of a variety of machine learning regression methods can be utilized as appropriate to the requirements of specific applications in accordance with various embodiments of the invention.

Many embodiments include OrbNet processes that incorporate AO-based feature databases to determine accurate molecular system properties. Several embodiments use databases of arbitrary molecular systems with their associated properties and the difference between the molecular properties as a training set to regress a model including (but not limited to) an OrbNet model for the molecular properties as a function of AO-based features and/or other features. Some embodiments rank and/or order candidate molecules on the basis of the trained model(s). Certain embodiments classify and/or sort candidate molecules on the basis of the trained model(s). A number of embodiments propose candidate molecules and then optimize them on the basis of the trained model(s). Several embodiments invert the trained model(s) to predict AO-based feature values including (but not limited to) SAAO feature values that can lead to desired values of the molecular properties. Many embodiments implement the inverted model(s) to optimize, rank, sort, classify and/or predict molecules with desired molecular properties. Examples of such properties include (but are not limited to) solubility, binding affinity, binding affinity for proteins, redox potential, pKa, electrical conductivity, ionic conductivity, thermal conductivity, light absorption frequency, light absorption intensity, and light absorption efficiency.

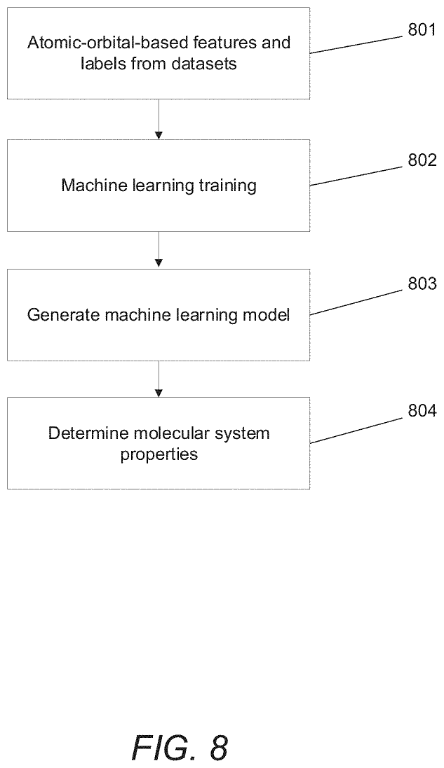

Examples of such embodiments are illustrated in FIG. 8. AO-based features and labels from accurate reference calculations can be extracted from the AO databases 801. Many embodiments use the AOs for evaluating matrix elements of the operators for feature generation. A machine learning model can be trained based on the selected AO-based features including (but not limited to) SAAO features 802. A trained model can be used to predict the labels from these features 803 and/or can be utilized in generative processes. The model may be used to predict accurate molecular system properties including (but not limited to) SAAO-resolved properties, whole molecule properties, and quantum mechanical energies 804. Such embodiments of machine learning regression can include but are not limited to: graph neural network (GNN). Some embodiments implement a GNN with a multi head graph attention mechanism and/or performer attention mechanism and residual blocks to improve the representation capacity to learn complex chemical environments. As can readily be appreciated, any of a variety of machine learning regression processes can be utilized as appropriate to the requirements of specific applications in accordance with various embodiments of the invention.

In many embodiments, the molecular system properties that are determined using the OrbNet process include but are not limited to AO-pair contributions to correlation energies, quantum mechanical energies, forces, vibrational frequencies (hessian), dipole moments, response properties, excited state energies and forces, inter atomic forces, optimized geometries, and spectra. As can readily be appreciated, any of a variety of molecular system properties can be utilized as appropriate to the requirements of specific applications in accordance with various embodiments of the invention. Some embodiments implement the prediction of forces and hessians that can be used to optimize the geometry of the molecular system to a local minimum or saddle point. Several embodiments include that the prediction of forces can be used to run molecular dynamics. Yet some embodiments include the prediction of energies and forces that can be used to perform configurational sampling. The predictions, according to several embodiments, can be made for high-level theories on the basis of AO-based feature values that are obtained using one level of electronic structure theory. Examples of high-level theories can include (but not limited to) DFT with a hybrid exchange correlation functional. As can readily be appreciated, the specific features used as high-level theories are largely only limited to the requirements of specific applications. In some embodiments, the prediction can be made for large basis set on the basis of AO-based feature values that may include data from a small basis set. Examples of a small basis set can include (but are not limited to) a minimal basis set. As can readily be appreciated, the specific features used as small basis set are largely only limited to the requirements of specific applications. Examples of a large basis set can include (but are not limited to) a different and larger basis set compared to the small basis set. As can readily be appreciated, the specific features used as large basis set are largely only limited to the requirements of specific applications.

As the amount of quantum simulation data increases, OrbNet processes in accordance with many embodiments of the invention can utilize online learning techniques to continuously update OrbNet models without retraining the models using the entirety of the original training data set. As can readily be appreciated, any of a variety of online ML techniques can be utilized to update previously trained OrbNet models using additional quantum simulation data as appropriate to the requirements of specific applications in accordance with various embodiments of the invention. In several embodiments, software implementations of OrbNet models can provide user interfaces that enable a user to efficiently update an existing OrbNet model using additional sources of quantum simulation data selected by the user including (but not limited) streams of quantum simulation data.

While various processes for machine learning regression are described above, any variety of machine learning regression methods can be utilized in ML processes as appropriate to the requirements of specific applications in accordance with various embodiments of the invention including (but not limited to) ML processes that are trained using graph representations of quantum chemical information (see discussion above). Processes for molecular synthesis in accordance with various embodiments of the invention are discussed further below.

### Molecular Synthesis

Processes in accordance with various embodiments of the invention can be utilized to synthesize molecules. In several embodiments, OrbNet processes are utilized to conduct a virtual screen of a set of candidate molecular systems based upon a set of one or more criteria related to chemical properties predicted by the OrbNet model. In a number of embodiments, a molecular system is identified using an inverse design or generative process in which a search of an AO-based feature space (or a suitable embedding thereof) is performed based upon a set of one or more criteria related to chemical properties predicted by OrbNet. Sets of AO-based features including (but not limited to) SAAO features that are predicted to possess desirable chemical properties by the OrbNet model can then be utilized to identify molecular structures corresponding to the AO-based features that are likely to possess the desired chemical properties. As is discussed further below, any of a variety of chemical property criteria can be utilized to perform virtual screening and/or inverse molecular design as appropriate to the requirements of specific applications in accordance with various embodiments of the invention.

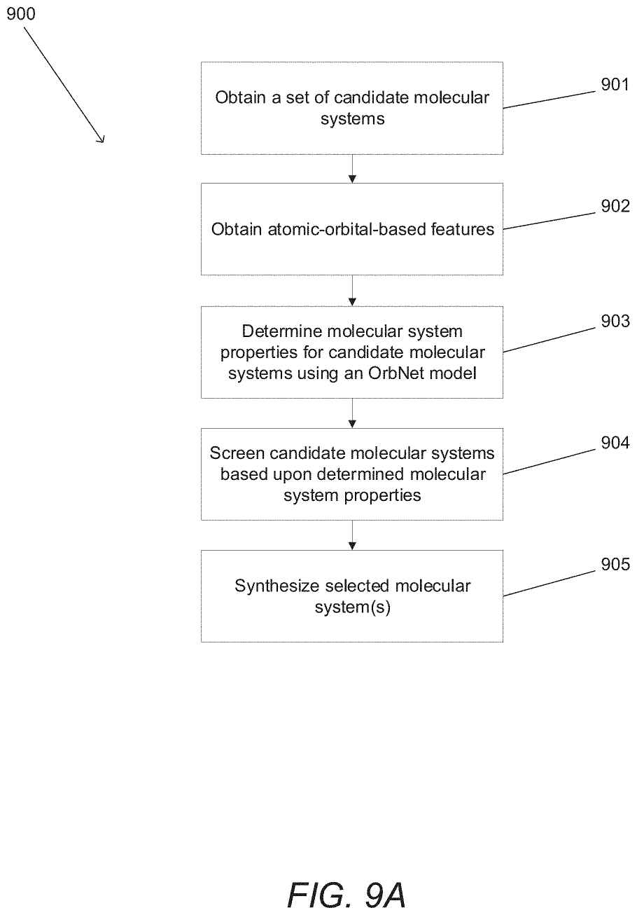

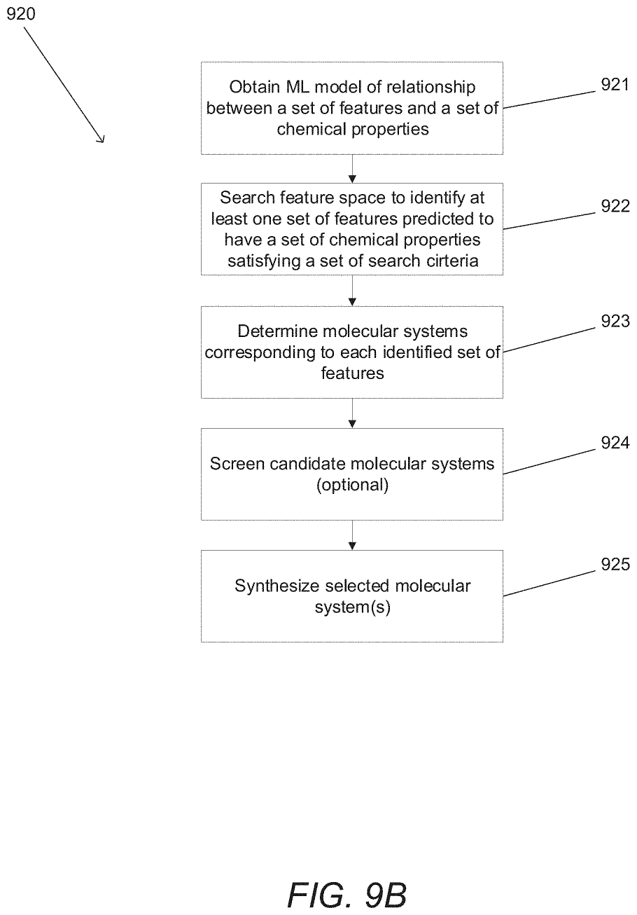

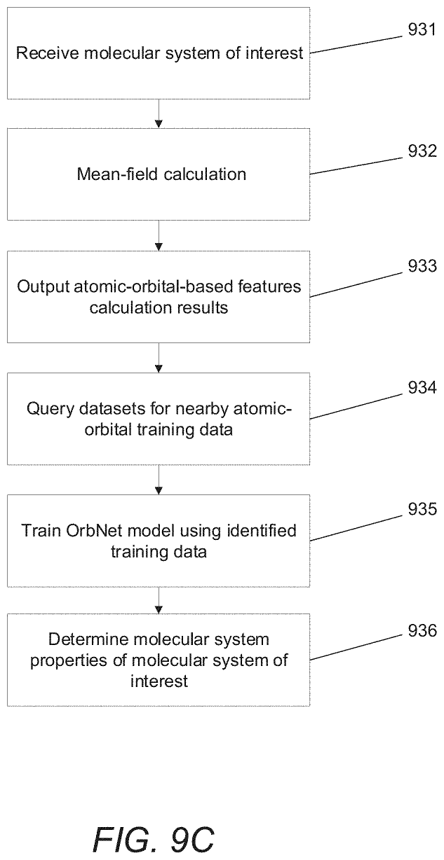

Many embodiments implement OrbNet processes that screen a set of candidate molecular systems based upon a set of criteria related to one or more desirable chemical properties to identify a molecular structure to synthesize. A method for screening candidate molecular systems molecules using an OrbNet process as part of a process for synthesizing a molecular system having a set of desirable characteristics in accordance with an embodiment of the invention is illustrated in FIG. 9A. The process 900 includes obtaining (901) a set of candidate molecular systems that are provided as inputs to the virtual screening process. In several embodiments, a quantum chemistry representation of the candidate molecular systems is obtained. In the illustrated embodiment, the candidate molecular systems are described (902) by a set of atomic-orbital-based features.

In several embodiments, an ML model that estimates one or more chemical properties based upon a quantum chemistry representation of a molecular system can be utilized in the virtual screening of the set of candidate molecular systems. In the illustrated embodiment, molecular system properties for the candidate molecular systems are predicted (903) using an OrbNet model trained using a process similar to any of the various processes described above. As can readily be appreciated, the specific ML model depends largely upon the quantum chemistry representation utilized to represent the candidate molecular systems, any processes utilized to reduce the dimensionality of the feature space of the quantum chemistry representation, the specific chemical properties predicted by the ML model, and/or the requirements of specific applications.

Predicted chemical properties of candidate molecular systems can be utilized to screen the candidate molecular systems in accordance with one or more criteria related to a desirable set of molecular system chemical properties. In many embodiments, additional criteria can also be utilized as part of the screen including known chemical properties of particular molecular systems such as (but not limited to) water solubility and/or toxicity. In several embodiments, the synthesis process can also further optimize the chemical structure of an identified molecular system to further enhance one or more desirable chemical properties. As can readily be appreciated, decreasing an undesirable chemical property can be treated in an equivalent manner to increasing a desirable chemical property. The candidate molecular system(s) determined to satisfy the set of criteria of the screening process can be output as report information, and/or synthesized (905).

While many quantum chemistry ML processes utilize candidate molecular systems as a starting point, the process of training a ML model based upon features derived from quantum chemistry information can inherently define a feature space that can be used for inverse molecule design. Accordingly, systems and methods in accordance with many embodiments of the invention utilize a quantum chemistry feature space to identify sets of quantum chemistry features that are likely to result in a molecular system with desirable chemical properties, and then identify molecular systems corresponding to the identified set of quantum chemistry features.

A process for synthesizing a molecular system having a desired set of chemical properties identified using an inverse molecule design process in accordance with an embodiment of the invention is illustrated in FIG. 9B. The process 920 includes obtaining (921) a ML model that describes the relationship between a set of features and a set of chemical properties. As can readily be appreciated, an OrbNet model can be utilized that is obtained using a process similar to any of the variety of processes for training OrbNet models described above. In a number of embodiments, an ML model trained based upon alternative quantum chemistry representations of molecular systems including (but not limited to) attributed graph representations can also be utilized. As can readily be appreciated, the specific ML model that is utilized depends largely upon the requirements of a particular application.

A search (922) can then be performed within the feature space of the ML model to identify sets of features that the ML model predicts will have a set of chemical properties that satisfy a set of search criteria.

As can readily be appreciated, the feature space corresponds to quantum chemical representations of molecular systems. Therefore, the inverse molecular design process involves identification (923) of a molecular system possessing a quantum chemical representation corresponding to the identified set of features. In a number of embodiments, the mapping of a set of features in the feature space of the ML model to a molecular system can be achieved using a feature-structure map. In several embodiments, the feature-structure map can be learned from a set of training data in which molecular structures with bonding information and/or any other atomic representations are annotated with sets of features in the feature space. As can readily be appreciated, any of a variety of training data sets and/or machine learning processes can be utilized to learn a process for mapping from a feature space to specific molecular structures.

In a number of embodiments, the inverse molecule design process yields a set of candidate molecular systems with predicted chemical properties. An addition screen can be performed (924) to filter the list of candidate molecular systems based upon a variety of criteria including (but not limited to): complexity of chemical synthesis, known toxicity, water solubility, and/or any of a variety of alternative chemical properties. When an appropriate candidate molecular system is identified, a report can be generated and/or the selected molecular system synthesized (925).

While various processes for identifying molecular structures for synthesis are described above, any of a variety of processes that identify molecular structures using ML models can be utilized to perform chemical synthesis as appropriate to the requirements of specific applications in accordance with various embodiments of the invention. ML processes can also be utilized in the context of quantum chemistry calculations for a variety of additional purposes. Processes for using ML in quantum chemistry calculations in accordance with various embodiments of the invention are discussed further below.

### Molecular “Fitting Room”

In a number of embodiments, a particular molecular system of interest can be utilized to identify a set of relevant AO-based feature training data from a database of molecular systems for which chemical properties are known. The database of molecular systems can be queried to identify AOs based upon distance in feature space between AOs represented within the database and AOs of the molecular system of interest. A distance metric can be utilized to measure the distance between AO-based features of molecules in the database and the AO-based features of the molecular system of interest. In this way, a molecular-system-specific training data set can be generated for the purposes of training an OrbNet model to predict the chemical properties (e.g. quantum mechanical energy) of the molecular system of interest.

A specific process for training an OrbNet model for estimating the chemical properties of a specific candidate molecular system in accordance with an embodiment of the invention is illustrated in FIG. 9C. The OrbNet process receives (931) as an input a specific molecular system. A set of AO-based features including (but not limited to) SAAO features for the molecular orbitals of the specific molecular system are generated. In the illustrated embodiment, the AO-based features are generated by performing (932) mean-field calculations and obtaining (933) AO-based features based upon the results of the calculations. The AO-based features can then be utilized to query (934) a database to identify AOs that are described within the database that are proximate in AO-based feature space to the AOs of the specific molecular system of interest. The AO-based features of the proximate AOs and their chemical properties can then be utilized to train (935) an OrbNet model that can then be utilized to accurately predict (936) the chemical properties of the specific molecular system that was the input of the process. As can readily be appreciated, training OrbNet models in the specific region in feature space occupied by a particular specific molecular system can greatly increase the accuracy with which estimates can be made of the chemical properties of that specific molecular system.

While the discussion of the processes described above with reference to FIG. 9C largely focus on processes for identifying training data in an AO-based feature space, similar processes can be performed using any of a variety of AO-based representations of molecular systems including (but not limited to) attributed graph representations. Systems and methods that utilize ML processes and ML models similar to those described above to provide quantum chemistry calculations for specific molecular systems are discussed further below.

### Quantum Chemistry Program

Processes in accordance with various embodiments of the invention rely upon quantum chemistry properties. As is discussed further below, any of a variety of quantum chemistry predictions of AO-based features of different molecular systems can be utilized as appropriate to the requirements of specific applications in accordance with various embodiments of the invention.

Many embodiments implement physics-based quantum chemistry predictions as input AO-based features including (but not limited to) SAAO features of molecular systems during OrbNet processes. Several embodiments implement predictions of physics-based quantum chemistry for the molecular system on the basis of AO-based features. Some embodiments include that the output results can include molecular system properties. Various embodiments of quantum chemistry programs include (but are not limited to) coupled-cluster theory and density functional theory. As can readily be appreciated, the specific features used as quantum chemistry programs are largely only limited to the requirements of specific applications. Many embodiments are incorporated in software packages.

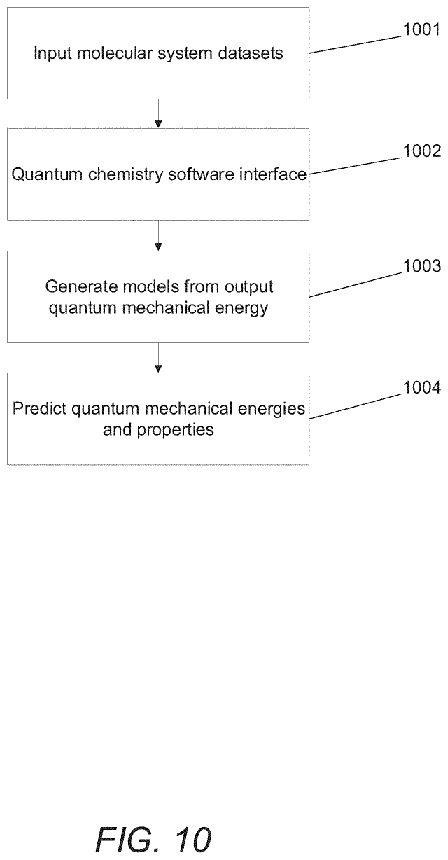

A system for incorporating an OrbNet process into a software package in accordance with an embodiment of the invention is illustrated in FIG. 10. A user can provide input to a quantum chemistry software package 1001. The user can perform physics-based calculations 1102. Results of the calculations can be replaced with the predictions of a ML model from the AO-based features corresponding to the user inputs 1003. Generalizations can include accelerating rather than replacing physics-based calculations using models based on AO-based features to predict intermediate quantities 1004; and generation of the machine learned model using these strategies.

In some embodiments, software packages incorporating OrbNet processes can be operated on a user-friendly platform, examples of such embodiments include (but are not limited to): smart phones, tablets, and computers. As can readily be appreciated, the specific features used as user platforms are largely only limited to the requirements of specific applications. According to some embodiments, the software package performs quantum simulations in seconds via a backend cloud-based deployment of OrbNet processes.

While various processes for generating quantum chemistry predictions from AO-based features are described above, any variety of processes that predict molecular system properties based on AO-based features can be utilized in OrbNet processes as appropriate to the requirements of specific applications in accordance with various embodiments of the invention. Various examples implementing OrbNet processes in accordance with various embodiments of the invention are discussed further below.

### Exemplary Embodiments

The following section provides specific examples of the use of different OrbNet processes to determine molecular compositions and structures for synthesis. Examples 1-9 implement OrbNet processes with SAAO features. Examples 10-13 implement OrbNet processes with AO features. As can readily be appreciated, OrbNet processes can be implemented in any of a variety of different ways and/or using any of a variety of different software packages. It will be understood that the specific embodiments are provided for exemplary purposes and are not limiting to the overall scope of the disclosure, which must be considered in light of the entire specification, figures and claims.

### Computational Details of Examples 1 and 2

Examples 1 and 2 use the QM7b-T (a thermalized version of the QM7b set of 7211 molecules with up to seven C, O, N, S, and CI heavy atoms) and the GDB-13-T (a thermalized version of the GDB-13 set of molecules with thirteen C, O, N, S, and CI heavy atoms). For these datasets, training and test geometries are sampled at 50 fs intervals from ab initio molecular dynamics trajectories performed using the B3LYP/6-31g* level of theory and a Langevin thermostat at 350 K.

Minimal-basis Hartree-Fock (HF) calculations are performed using the STO-3G AO basis. Large-basis HF calculations are performed using the cc-pVTZ AO basis. And semi-empirical xtb calculations are performed using the non-self-consistent GFN0-xTB method. These calculations and the corresponding generation of SAAOs are performed using the ENTOS QCORE package. For DFT label values, the ωB97X-D functional is employed in a Def2-TZVP AO basis set; these calculations are also performed using ENTOS QCORE.

Density fitting for both Coulomb and exchange integrals is employed for Hartree-Fock and DFT results in Examples 1 and 2. The frozen core approximation is used in all cases.

### Example 1: Minimal Basis to Large Basis HF Energy with OrbNet of SAAO Features

Many embodiments implement OrbNet to predict the large basis set (i.e., cc-pVTZ) Hartree-Fock (HF) energy of the molecular system from features computed using a cheap minimal-basis (i.e., STO-3G) HF calculation. The regression labels are the difference between the large-basis and the small-basis HF atomization energies, i.e.

ΔE^{ML}≈(E^{TZ}−E_{free}^{TZ})−(E^{SZ}−E_{free}^{SZ}) (44)

where E^{TZ} and E^{SZ} denote the HF energy obtained from the large and minimal basis set; E_{free}^{TZ} and E_{free}^{SZ} denote the summation of ground-state free atom energies of the molecule obtained from the large and minimal basis set, respectively.

The accuracy of the ML predictions is reported in Table 1. Table 1 includes MAE results for learning the STO-3G to predict the cc-pVTZ HF atomization targets, using F, D, and P under the SAAO basis for graph featurization. The model is trained on 6500 QM7b-T molecules, and results are reported from models trained using either 1 or 7 thermally sampled geometries for each molecule. Chemical accuracy is reached for the normalized MAE on both QM7b-T and GDB-13-T.

TABLE 1
MAE results for learning the STO-3G to predict the cc-pVTZ
HF atomization targets.

| Training size | QM7b-T MAE (mH) | GDB13-T MAE/7HA (mH) |
|---|---|---|
| 6500*1geo | 1.776 | 2.075 |
| 6500*7geo | 0.861 | 1.557 |

### Examples 2: xTB to DFT Energy with OrbNet of SAAO Features

Many embodiments implement OrbNet to predict the energy of a high-level theory (i.e., DFT with the ωB97X-D range-separated hybrid functional and Def2-TZVP AO basis) of the molecular system from features computed using a low-computational-cost semi-empirical method (i.e., GFN0-xTB). As GFN0-xTB is a non-self-consistent field-based method, features are obtained with a small pre-factor for the O(N³) operation while avoiding the possibility of convergence difficulties that can plague large molecular systems. The regression labels are the difference between the high-level DFT and the GFN0-xTB atomization energies, i.e.

ΔE^{ML}≈(E^{DFT}−E_{free}^{DFT})−(E^{xTB}−E_{free}^{xTB})−ΔE^{fit} (45)

where ΔE^{fit} is a correction term obtained from a linear fitting on the training set to the atomization energy difference with respect to the number of atoms in the molecule for each element.

The accuracy of the ML predictions is reported in Table 2. Table 2 includes MAE results for learning the GFN0-x TB to predict the ωB97X-D/Def2-TZVP DFT atomization targets, using F, J, K, D, and P under the SAAO basis for graph featurization. The model is trained on 6500 QM7b-T molecules, and results are reported from models trained using either 1 or 7 thermally sampled geometries for each molecule. Cost reduction is about 1000-fold or more of computing features from GFN0-xTB calculations in comparison to the full computational cost of the popular ωB97X-D/Def2-TZVP level of theory.

TABLE 2
MAE results for learning the GFN0-x TB to predict the
ωB97X-D/Def2-TZVP DFT atomization targets.

| Training size | QM7b-T MAE (mH) | GDB13-T MAE/7HA (mH) |
|---|---|---|
| 6500*1geo | 2.151 | 3.066 |
| 6500*7geo | 1.329 | 2.520 |

### Computational Details of Examples 3 and 4

Examples 3-4 implement the following datasets: the QM7b-T dataset (which has seven conformations for each of 7211 molecules with up to seven heavy atoms of type C, O, N, S, and Cl), the QM9 dataset (which has locally optimized geometries for 133885 molecules with up to nine heavy atoms of type C, O, N, and F), the GDB-13-T dataset (which has six conformations for each of 1000 molecules from the GDB-13 dataset with up to thirteen heavy atoms of type C, O, N, S, and Cl), DrugBank-T (which has six conformations for each of 168 molecules from the DrugBank database with between fourteen and 30 heavy atoms of type C, O, N, S, and Cl), and the Hutchison conformer dataset (which has up to 10 conformations for each of 622 molecules with between nine and 50 heavy atoms of type C, O, N, F, P, S, CI, Br, and I). Thermalized geometries from the DrugBank dataset can be sampled at 50 fs intervals from ab initio molecular dynamics trajectories performed using the B3LYP/6-31g level of theory and a Langevin thermostat at 350 K. For results reported in Example 3, the pre-computed DFT labels from Ramakrishnan et al. are employed. (See, e.g., R. Ramakrishnan, et al., Sci. Data, 2014, 1, 1-7; the disclosure of which is incorporated herein by reference.) For results reported in Example 4, all DFT labels can be computed using the ωB97X-D functional with a Def2-TZVP AO basis set and using density fitting for both the Coulomb and exchange integrals using the Def2-Universal-JKFIT basis set; these calculations are performed using PSI4. Semi-empirical calculations are performed using the GFN1-xTB method using the ENTOS QCORE package, which is also employed for the SAAO feature generation.

For the results in Examples 3-4, OrbNet models can be trained using the following training-test splits of the datasets. For results on the QM9 dataset, 3054 molecules are removed due to a failed a geometric consistency check. Then 110000 molecules are randomly sampled for training and used 10831 molecules for testing. The training sets of 25000 and 50000 molecules in Example 3 are subsampled from the 110000-molecule dataset. For the QM7b-T dataset, two sets of training-test splits are generated; for the model trained on the QM7b-T dataset only (Model 1 in Example 4), 6500 different molecules (with 7 geometries for each) are randomly selected from the total 7211 molecules for training, holding out 500 molecules (with 7 geometries for each) for testing; for Models 2-4 in Example 4, a 361-molecule subset of this 500-molecules set is used for testing, and the remaining 6850 molecules of QM7b-T are used for training. For the GDB13-T dataset, 948 different molecules (with 6 geometries for each) are randomly sampled for training, holding out 48 molecules (with 6 geometries for each) for testing. For the DrugBank-T dataset, 158 different molecules (with 6 geometries for each) are randomly sampled for training, holding out 10 molecules (with 6 geometries for each) for testing. No training on the Hutchison conformer dataset is performed. Since none of the training datasets for OrbNet includes molecules with elements of type P, Br, and I, the molecules in the Hutchison dataset that included elements of these types are excluded. Sixteen molecules are excluded due to missing DLPNO-LCCSD(T) reference data; an additional eight molecules are excluded on the basis of DFT convergence issues for at least one conformer using PSI 4.

Table 3 summarizes the hyperparameters used for training OrbNet for the results in Example 3 and 4. A pre-transformation on the input features is performed from F, J, K, D, P, H, and S to obtain \tilde{X} and \tilde{X}^{e}: all diagonal SAAO tensor values Xᵤᵤ are normalized to range [0, 1) for each operator type to obtain \tilde{X}ᵤ; for off-diagonal SAAO tensor values, \tilde{X}ᵤᵥ=−ln(|Xᵤᵥ|) is taken for X∈F,J,K,P,S,H, and \tilde{D}ᵤᵥ=Dᵤᵥ. The model hyperparameters are selected within a limited search space; the cutoff hyperparameters c_{X} are obtained by examining the overlap between feature element distributions between the QM7b-T and GDB13-T datasets. The same set of hyperparameters is used throughout Example 3 and 4.

TABLE 3
Model hyperparameters employed in OrbNet in Examples 3 and 4.
All cutoff values are in atomic units.

| Hyper- | | Value or |
|---|---|---|
| parameter | Meaning | name |
| nᵣ | Number of basis functions for node | 8 |
| | embedding | |
| mᵣ | Number of basis functions for edge | 8 |
| | embedding | |
| nₕ | Dimension of hidden node attributes | 256 |
| nₑ | Dimension of hidden edge embedding | 64 |
| nₐ | Number of attention heads | 4 |
| L | Number of message passing layers | 3 |
| L_{enc} | Number of dense layers in Encₕ and Encₑ | 3 |
| L_{dec} | Number of dense layers in a decoding | 4 |
| | network | |
| σ | Activation function | Swish |
| σₐ | Activation function for attention | TanhShrink |
| | generation | |
| γ | Batch normalization momentum | 0.4 |
| c_{F} | Cutoff value for [symbol] | 8.0 |
| c_{J} | Cutoff value for [symbol] | 1.6 |
| c_{K} | Cutoff value for [symbol] | 20.0 |
| c_{D} | Cutoff value for [symbol] | 9.45 |
| c_{P} | Cutoff value for [symbol] | 14.0 |
| c_{S} | Cutoff value for [symbol] | 8.0 |
| c_{H} | Cutoff value for [symbol] | 8.0 |

To provide additional regularization for predicting energy variations from the configurational degree of freedom, training is performed on loss function of the form

$$ℒ ( \hat{E} , E ) = ( 1 - α ) {\sum }_{i} {ℒ}_{2} ( {\hat{E}}_{i} , {E}_{i} ) + α {\sum }_{i} {ℒ}_{2} ( {\hat{E}}_{ι} - {\hat{E}}_{t ( i )} , {E}_{i} - {E}_{t ( i )} ) \qquad ( 46 )$$

For a conformer i in a minibatch, another conformer t(i) of the same molecule is randomly sampled to be paired with i to evaluate the relative conformer loss [symbol] ₂(Êᵢ−Eₜ₍ᵢ₎,Eᵢ−Eₜ₍ᵢ₎), putting additional penalty on the prediction errors for configurational energy variations. E denotes the ground truth energy values of the minibatch, E denotes the model prediction values of the minibatch, and [symbol] ₂ denotes the L2 loss function [symbol] ₂(ŷ, y)=∥ŷ−y∥₂². For all models in Example 3, α=0 is used as only the optimized geometries are available; for models in Example 4, α=0.9 is used for all training setups.

All models in Example 3 and 4 are trained on a single Nvidia Tesla V100-SXM2-32 GB GPU using the Adam optimizer. For all training runs, the minibatch size is set to 64 and use a cyclical learning rate schedule that performs a linear learning rate increase from 3×10⁻⁵ to 3×10⁻³ for the initial 100 epochs, a linear decay from 3×10⁻³ to 3×10⁻⁵ for the next 100 epochs, and an exponential decay with a factor of 0.9 every epoch for the final 100 epochs. Batch normalization is employed before every activation function a except for that used in the attention heads, σₐ.

### Example 3: The QM9 Formation Energy with OrbNet of SAAO Features

Many embodiments provide the prediction of accurate DFT energies using input features obtained from the GFN1-xTB method. The GFN family of methods can be useful for the simulation of large molecular system (1000s of atoms or more) with time-to-solution for energies and forces on the order of seconds. However, this applicability can be limited by the accuracy of the semi-empirical method, thus creating a natural opportunity for “delta-learning” the difference between the GFN1 and DFT energies on the basis of the GFN1 features. In several embodiments, regression labels can be associated with the difference between high-level DFT and the GFN1-xTB total atomization energies,

E^{ML}≈E^{DFT}−E^{GFN1}−ΔEₐₜₒₘₛ^{fit} (47)

where the last term is the sum of differences for the isolated-atom energies between DFT and GFN1 as determined by a linear model. This approach yields the direct ML prediction of total DFT energies, given the results of a GFN1-xTB calculation.

Many embodiments compare OrbNet process with other ML methods in predicting the total energy task, U₀, from the QM9 dataset. QM9 is composed of organic molecules with up to nine heavy atoms at locally optimized geometries. This test examines the expressive power of the ML models for systems in similar chemical environments. Results for OrbNet are presented both without ensemble averaging of independently trained models (i.e., predicting only on the basis of the first of trained model) and with ensemble averaging the results of five independently trained models (OrbNet-ens5). Ensembling of OrbNet in accordance with some embodiments may help reduce the OrbNet prediction error by about 10% to about 20%. Several embodiments implement OrbNet with multi-task learning. OrbNet with multi-task learning is trained with both molecular energies and other computed properties of the quantum mechanical wavefunction. The learning efficiency can be improved by incorporating physically motivated constraints on the electronic structure through multi-task learning. OrbNet with multi-task learning shows improved accuracy on energy prediction tasks for the QM9 dataset, at a computational cost that is thousand-fold or more reduced compared to conventional quantum chemistry calculations (such as density functional theory) that offer similar accuracy. Prediction results of the QM9 dataset from methods utilizing graph representations of atom-based features, including SchNet, PhysNet, DimeNet, and DeepMoleNet are provided. (See, e.g. K. Schutt, et al., Advances in neural information processing systems, 2017, 991-1001; O. T. Unke, et al., J. Chem. Theory Comput., 2019, 15, 3678-3693; J. Klcpera, et al., International Conference on Learning Representations, 2019; the disclosures of which are incorporated herein by references.) DimeNet employs a directional message passing mechanism and PhysNet and DeepMoleNet employ supervision based on prior physical information to improve the model transferability. Many embodiments provide that OrbNet provides greater accuracy and learning efficiency than all previous deep-learning methods.

Table 4 lists MAEs (in meV) for predicting the QM9 dataset of total energies at the B3LYP/6-31G (2df,p) level of theory. Results are listed for a single model (OrbNet), ensembling over 5 models (OrbNet-ens5), OrbNet with multi-task learning (OrbNet-multi), SchNet, PhysNet, DimeNet, and DeepMoleNet.

TABLE 4
MAEs for predicting the QM9 dataset of total energies for different ML models

| Training | | | PhysNet- | | | | OrbNet- | OrbNet- |
|---|---|---|---|---|---|---|---|---|
| size | SchNet | PhysNet | ens5 | DimeNet | DeepMoleNet | OrbNet | ens5 | multi |
| 25,000 | — | — | — | — | — | 11.6 | 10.4 | 8.08 |
| 50,000 | 15 | 13 | 10 | — | — | 8.22 | 6.80 | 5.89 |
| 110,000 | 14 | 8.2 | 6.1 | 8.02 | 6.1 | 5.01 | 3.92 | 3.87 |

### Example 4: Transferability and Conformer Energy Predictions with OrbNet of SAAO Features

Many embodiments provide transferability of OrbNet processes. In several embodiments, OrbNet is trained on datasets of relatively small molecules (for which high-accuracy data is more readily available) and then tested on datasets of larger and more diverse molecules. Some embodiments provide the performance of OrbNet on a series of datasets containing organic and drug-like molecules.

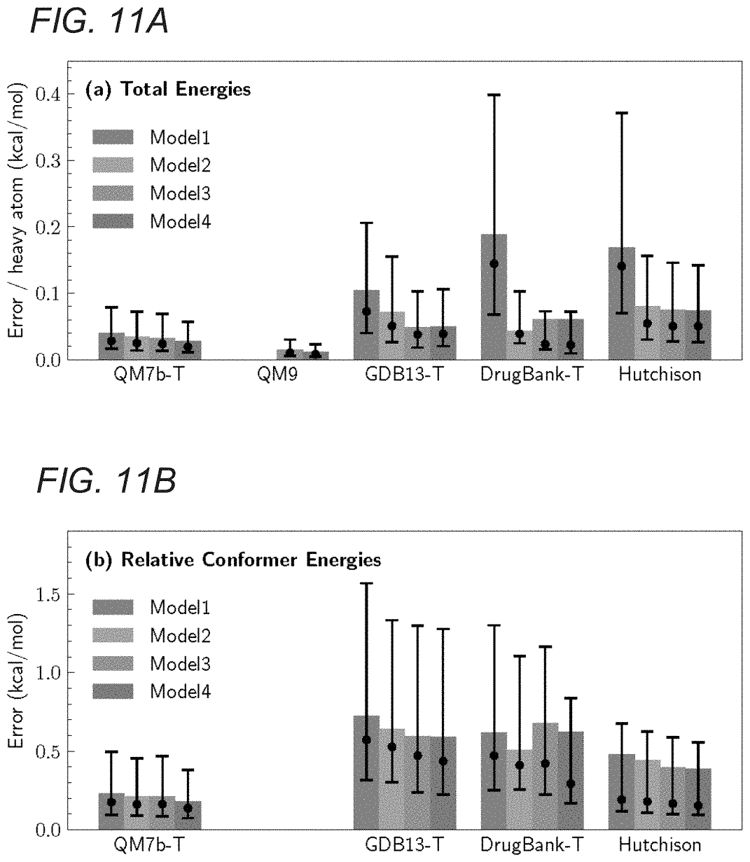

Prediction errors for molecular total energies and relative conformer energies using OrbNet models in accordance with an embodiment of the invention are illustrated in FIGS. 11A and 11B respectively. In FIGS. 11A and 11B, OrbNet models are trained with increasing amounts of data. The mean absolute error (MAE) is indicated by the bar height, the median of the absolute error is indicated by a black dot, and the first and third quantiles for the absolute error are indicated as the lower and upper bars. Using the training-test splits described in Computational Details in Example 3 and 4, Model 1 is trained using data from the QM7b-T dataset; Model 2 is trained using data from the QM7b-T, GDB13-T, and DrugBank-T datasets; Model 3 is trained using data from the QM7b-T, QM9, GDB13-T, and DrugBank-T datasets; and Model 4 is obtained by ensembling five independent training runs with the same data as used for Model 3. Predictions are made for total energies (FIG. 11A) and relative conformer energies (FIG. 11B) for held-out molecules from each of these datasets, as well as for the Hutchison conformer dataset. Training and prediction employ energies at the ωB97X-D/Def2-TZVP level of theory. All energies in kcal/mol.

OrbNet predictions improve with additional data and with ensemble modeling. The median and mean of the absolute errors consistently decrease from Model 1 to Model 4 except for a non-monotonicity in the DrugBank-T MAE, likely due to the relatively small size of that dataset. FIG. 11B shows that Model 1, which includes only data from QM7b-T yields relative conformer energy predictions on the DrugBank-T and Hutchison datasets (which include molecules with up to 50 heavy atoms) with an accuracy that is comparable to the more heavily trained models. All of the OrbNet models predict relative conformer energies with MAE and median prediction errors that are well within the 1 kcal/mol threshold of chemical accuracy, across all four test datasets. Predictions for QM9 using Models 1 and 2 are not included, since QM9 includes F atoms whereas the training data in those models do not; relative conformer energies are not predicted for QM9 since they are not available in this dataset. Although total energy prediction error for OrbNet is slightly larger per heavy atom on the Hutchison dataset than for the other datasets, the relative conformer energy prediction error for the Hutchison dataset is slightly smaller than for GDB13-T and DrugBank-T. This might be due to the fact that the Hutchison dataset involves locally minimized conformers that are less spread in energy per heavy atom than the conformers of the thermalized datasets.

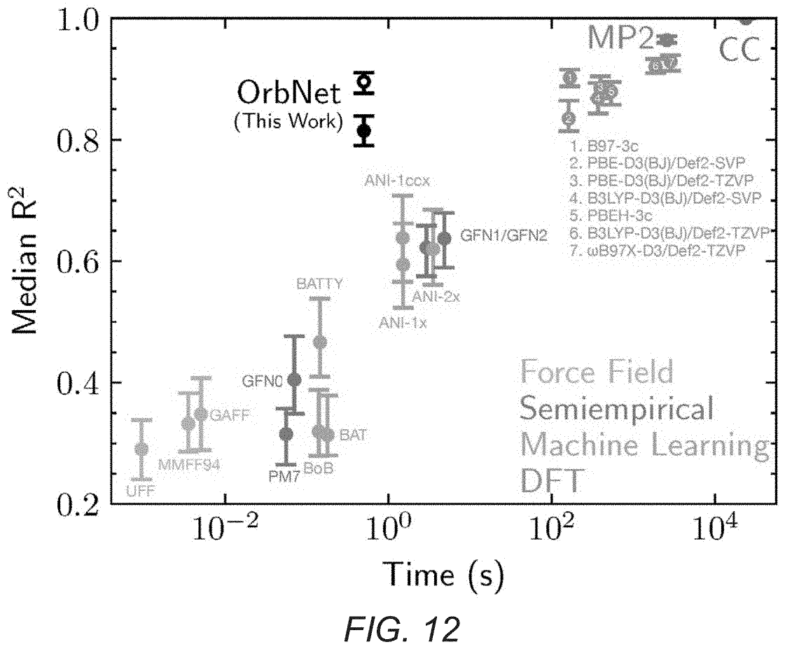

Comparison of the accuracy and computational-cost tradeoff for a range of potential energy methods for the Hutchison conformer benchmark dataset in accordance with an embodiment of the invention is illustrated in FIG. 12. FIG. 12 presents a direct comparison of the accuracy and computational cost of OrbNet in comparison to a variety of other force-field, semi-empirical, machine-learning, DFT, and wavefunction methods. For the Hutchison conformer dataset of drug-like molecules which range in size from nine to 50 heavy atoms, the accuracy of the various methods is evaluated using the median R² of the predicted conformer energies in comparison to DLPNO-CCSD(T) reference data and with computation time evaluated on a single CPU core.

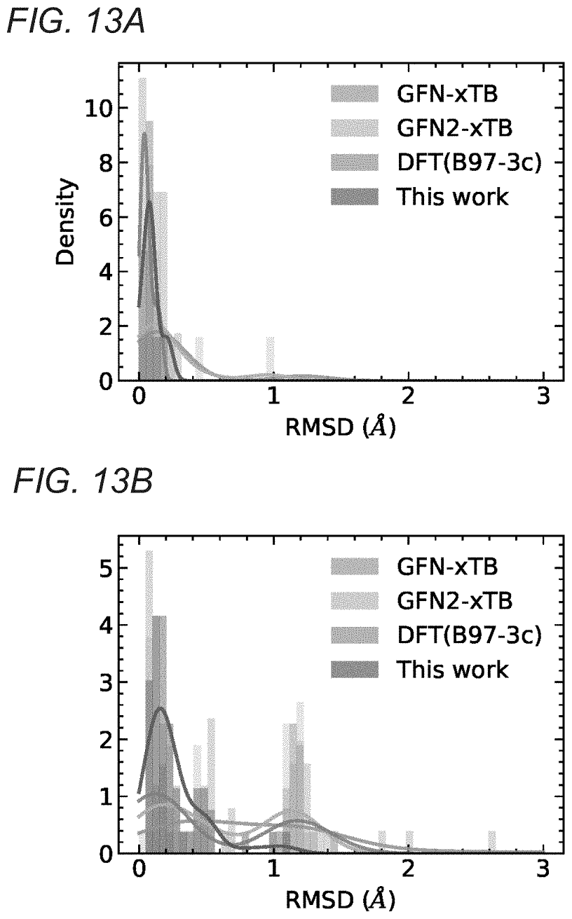

The OrbNet conformer energy predictions in FIG. 12 are reported using Model 4 (i.e., with training data from QM7b-T, GDB13-T, DrugBank-T, and QM9 and with ensemble averaging over five independent training runs). The solid black circle indicates the median R² value (0.81) of the OrbNet predictions relative to the DLPNO-CCSD(T) reference data, as for the other methods; this point provides a direct comparison to the accuracy of the other methods. The open black circle indicates the median R² value (0.90) of the OrbNet predictions relative to the ωB97X-D/Def2-TZVP reference data against which the model is trained; this point indicates the accuracy that would be expected of the Model 4 implementation of OrbNet if it has employed coupled-cluster training data rather than DFT training data. Error bars correspond to the 95% confidence interval, determined by statistical bootstrapping. We performed timings for OrbNet on a single core of an Intel™ Core i5-1038NG7 CPU @ 2.00 GHz, finding that the OrbNet computational cost is dominated by the GFN1-xTB calculation for the feature generation. OrbNet uses ENTOS QCORE for the GFN1-xTB calculation calculations. The reported timings for GFN1-xTB to be slow in Hutchison, particularly in comparison to the GFN0-xTB timings. (See, e.g., G. Hutchison, et al., Int. j. Quantum Chem., 2020, e26381; the disclosure of which is incorporated herein by reference.) For GFN0-xTB, timings with ENTOS QCORE are similar to those reported in Hutchison, which is sensible given that the method involves no self-consistent field (SCF) iteration. However, whereas Hutchison indicates GFN1-xTB timings that are 43-fold slower than GFN0-xTB, OrbNet shows this ratio to be about 4.5 with ENTOS QCORE. To account for the issue of code efficiency in the GFN1-xTB implementation and to control for the details of the single CPU core used in the timings versus in Hutchison, the OrbNet timing is normalized in FIG. 13 with respect to the GFN0-xTB timing from Hutchison. The CPU neural-network inference costs for OrbNet are negligible contribution to this timing.

Many embodiments show that OrbNet enables the prediction of relative conformer energies for drug-like molecules with an accuracy that is comparable to DFT but with a computational cost that is 1000-fold reduced from DFT to realm of semi-empirical methods. Several embodiments indicate that OrbNet provides improvements in prediction accuracy over currently available ML and semi-empirical methods for realistic applications, without significant increases in computational cost.

### Computational Details of Example 5

Many embodiments provide that training of OrbNet in Example 5 includes optimized and thermalized geometries of molecules up to 30 heavy atoms from the QM7b-T, QM9, GDB13-T, and DrugBank-T datasets. Model training uses the dataset splits of Model 3 in Example 4. DFT labels are computed using the ωB97X-D3 functional with a Def2-TZVP AO basis set and using density fitting for both the Coulomb and exchange integrals using the Def2-Universal-JKFIT basis set.

For results in Example 5, geometry optimization is performed for the DFT, OrbNet, and GFN-xTB calculations by minimizing the potential energy using the BFGS algorithm with the Translation-rotation coordinates (TRIC); geometry optimizations for GFN2-xTB are performed using the default algorithm in the XTB package. All local geometry optimizations are initialized from pre-optimized structures at the ωB97X-D3/Def2-TZVP level of theory. For the B97-3c method, the mTZVP basis is employed.

All DFT and GFN-xTB calculations are performed using ENTOS QCORE; GFN2-xTB calculation are performed using XTB package.

### Example 5: Molecular Geometry Optimizations with OrbNet of SAAO Features

Several embodiments implement OrbNet with multi-task learning. OrbNet with multi-task learning is trained with both molecular energies and other computed properties of the quantum mechanical wavefunction. The learning efficiency can be improved by incorporating physically motivated constraints on the electronic structure through multi-task learning. OrbNet with multi-task learning model shows improved accuracy on molecular geometry optimizations on conformer datasets at a computational cost that is thousand-fold or more reduced compared to conventional quantum chemistry calculations (such as density functional theory) that offer similar accuracy.

A practical application of energy gradient (i.e., force) calculations is to optimize molecule structures by locally minimizing the energy. Many embodiments provide the accuracy of the OrbNet potential energy surface in comparison to other methods of comparable and greater computational cost. Test are performed for the ROT34 and MCONF datasets, with initial structures that are locally optimized at the high-quality level of ωB97X-D3/Def2-TZVP DFT with tight convergence parameters. ROT34 includes conformers of 12 small organic molecules with up to 13 heavy atoms; MCONF includes 52 conformers of the melatonin molecule which has 17 heavy atoms. From these initial structures, a local geometry optimization is performed using the various energy methods, including OrbNet, the GFN family of semi-empirical methods, and the relatively low-cost DFT functional B97-3c. The error in the resulting structure with respect to the reference structures optimized at the ωB97X-D3/Def2-TZVP level is computed as root mean squared distance (RMSD) following optimal molecular alignment. This test investigates whether the potential energy landscape for each method is locally consistent with a high-quality DFT description.

The molecular geometry optimization accuracy for the ROT34 and MCONF datasets, reported as the best-alignment root-mean-square-deviation (RMSD) compared to the reference DFT geometries at the ωB97X-D3/Def2-TZVP level in accordance with an embodiment of the invention is illustrated in FIG. 13A and FIG. 13B. The distribution of errors is plotted as histograms (with overlaying kernel density estimations). Timings correspond to the average cost for a single force evaluation for the MCONF dataset on a single Intel™ Xeon Gold 6130 @ 2.10 GHz CPU core. FIG. 13A and FIG. 13B show the resulting distribution of errors for the various methods over each dataset. Table 5 reports the mean errors and the percentage of optimized structures that correspond to incorrect geometries (i.e., RMSD>0.6 Angstrom). While the GFN semi-empirical methods provide a computational cost that is comparable to OrbNet, the resulting geometry optimizations are substantially less accurate, with a significant fraction of the local geometry optimizations relaxing into structures that are inconsistent with the optimized reference DFT structures (i.e., with RMSD in excess of 0.6 Angstrom). In comparison to DFT using the B97-3c functional, OrbNet provides optimized structures that are of comparable accuracy for ROT34 and that are more accurate for MCONF. However, OrbNet is over 100-fold less computationally expensive.

TABLE 5
The mean errors and the percentage of optimized structures that
correspond to incorrect geometries.

| | Mean RMSD (Å) | | Incorrect geometries | | Time/step |
|---|---|---|---|---|---|
| Method | ROT34 | MCONF | ROT34 | MCONF | MCONF |
| GFN-xTB | 0.23 | 0.90 | 8% | 52% | <1 s |
| GFN2-xTB | 0.21 | 0.60 | 8% | 44% | <1 s |
| DFT (B97-3c) | 0.06 | 0.51 | 0% | 37% | >100 s |
| OrbNet | 0.09 | 0.26 | 0% | 6% | <1 s |
| DFT (ωB97X-D3) | — | — | — | — | >1000 s |

### Computational Details of Examples 6-9

OrbNet Denali processes in accordance with several embodiments are implemented in Examples 6-9. OrbNet Denali processes have the following modifications compared to OrbNet processes in Examples 1-5: 1). The attention mechanism is replaced with performer attention. The performer attention mechanism can result in decreased memory use and negligible test accuracy degradation. 2). The number of message passing steps increases from 2 to 3. 3). Batch normalization layers are replaced with layer normalization layers. 4). The regression labels are modified to account for charged molecules. Examples 6-9 using OrbNet Denali model in accordance with several embodiments implement increased model and data scale which can lead to near-DFT performance. In some embodiments, OrbNet Denali model uses about 21 million trainable parameters and about 2.5 million training data.

Many embodiments provide a large collection of training data for OrbNet Denali in Examples 6-9. Some embodiments implement ChEMBL molecules in the training data. The ChEMBL27 database can be downloaded from the ChEMBL web service. Simplified molecular-input line-entry system (SMILES) strings containing 50 or fewer atoms of the elements C, O, N, F, S, Cl, Br, I, P, Si, B, Na, K, Li, Ca, or Mg and no isotope specifications are kept. SMILES strings that do not resolve to a closed-shell Lewis structure are discarded. All SMILES strings corresponding to molecules in the Hutchison conformer benchmark set are removed from the training dataset.

From this subset, a final surviving selection of 116,943 unique SMILES strings corresponding to neutral molecules are randomly chosen. Up to four conformers for each SMILES string are initially generated through the Entos Breeze conformer generator and optimized at the GFN1-xTB level. For each of these four energy-minimized conformers, non-equilibrium geometries are generated using Entos Breeze through either normal-mode sampling (NMS) at 300K or ab initio molecular dynamics (AIMD) sampling for 200 fs at 500k; in both cases at the GFN1-xTB level of theory. These thermalization methods are selected randomly with equal weight. This procedure results in a total of 1,771,191 equilibrium and non-equilibrium geometries.

Several embodiments implement protonation states and tautomers in training data. A subset of 26,186 SMILES strings are randomly chosen from the list of filtered ChEMBL SMILES strings. For each of these, up to unique 128 protonation states are identified using Dimorphite-DL version 1.2.4 and four of these protonation states are selected at random. The same conformer generation algorithm and non-equilibrium geometry sampling algorithms are applied to the four protonation states, resulting in a total of 215,866 unique geometries.

Some embodiments implement salt complexes and non-bonded interactions in training data. From the list of filtered ChEMBL SMILES strings, a number of SMILES strings are selected and randomly paired with between one to three salt molecules from the list of common salts in the ChEMBL Structure Pipeline. This procedure may result in a total of 21,735 salt complexes. For each of these complexes, four conformers are created through a conformer pipeline, and NMS sampling is used to generate four non-equilibrium geometries for each conformer. This resulted in 271,084 unique geometries. Additionally, the structures in the JSCH-2005 and the sidechain-sidechain interaction (SSI) subset of the BioFragment Database are added to the dataset.

Certain embodiments implement small molecules in the training data. A list of common chemical moieties and bonding patterns in organic molecules is created to avoid biasing the datasets to represent only large drug-like molecules, and used to enumerate the chemistry of small molecules with relatively “exotic” compositions, resulting in around 15,000 SMILES strings. For each of these, SMILES strings are created by randomly substituting hydrogen atoms for halogens, and carbon for silicon. This procedure can result in a total of 40,565 SMILES strings, for which conformers are generated through the conformers pipeline, resulting in a total of 94,588 unique geometries.

All DFT single-point calculations in Examples 6-9 are carried out in ENTOS QCORE version 0.8.17 at the ωB97X-D3/def2-TZVP level of theory using the in-core density fitting with the neese=4 DFT integration grid.

Many embodiments provide training details in Examples 6-9 for OrbNet Denali processes. PyTorch v1.7.1 and the Deep Graph Library (DGL) v0.6 are used to implement and train the model. PyTorch's Distributed Data Parallel (DDP) strategy is used to train the model on multiple GPUs using data parallelism. The OrbNet Denali model is trained on the OLCF Summit supercomputer using 96 NVIDIA V100-SXM2 (32G) GPUs with a batch size of 4 per GPU for 300 epochs, totaling 6912 GPU-hours of training. The learning rate is linearly warmed-up for the first 100 epochs and cosine annealed to zero for the remaining 200 epochs. During this process, the maximum learning rate is 3e-4. The Adam optimizer is used. The 1.8 TB dataset is randomly split into four shards. Each Summit node, comprising 6 GPUs, is assigned to one of these four shards such that each shard is used on ¼ of the nodes.

The regression labels in the OrbNet Denali model are described in Eq. 45. In Eq. 45, E^{DFT} is the reference DFT (i.e. ωB97X-D3/def2-TZVP) energy and E^{GFN1} is the GFN1-xTB energy. In OrbNet Denali model, ΔEₐₜₒₘₛ^{fit} is given by

$$Δ {E}_{a t o m s}^{f i t} = {\sum }_{i} Δ {E}_{{Z}_{i}}^{f i t} - Δ {E}_{{e}^{-}}^{f i t} q \qquad ( 48 )$$

where i indexes atoms within a molecule, Zᵢ is the atomic number of atom i, and q is the total charge of the molecule. ΔE_{Z}_{ᵢ}^{fit} and ΔEₑ_{^{−}}^{fit} are parameters and are fit to E^{DFT}−E^{GFN1} with ordinary least squares prior to OrbNet training.

OrbNet Denali 10% model in accordance with some embodiments is trained on 10% of the training data, sampled at random. All other training details are the same. Table 6 provides a comparison of models presented in Examples 6-9 to Examples 1-5.

TABLE 6
Comparison of OrbNet models in Examples 1-9

| | # of | # of | | |
|---|---|---|---|---|
| | trainable | training | Description of | Multi- |
| Model | parameters | data | training data | task? |
| OrbNet model in | ~1.8 million | ~0.2 million | QM7b-T, QM9, | No |
| Examples 1-4 | | | GDB-13-T, | |
| | | | DrugBank-T | |
| OrbNet model in | ~1.8 million | ~0.2 million | QM7b-T, QM9, | Yes |
| Example 5 | | | GDB-13-T, | |
| | | | DrugBank-T | |
| OrbNet Denali | ~21 million | ~0.2 million | 10% of training | No |
| 10% | | | data in | |
| | | | Computational | |
| | | | Details of | |
| | | | Examples 6-9 | |
| OrbNet Denali | ~21 million | ~2.5 million | Training data in | No |
| | | | Computational | |
| | | | Details of | |
| | | | Examples 6-9 | |

### Example 6: The GMTKN55 Collection with OrbNet of SAAO Features

The General Main-group Thermochemistry, Kinetics, and Non-covalent Interactions 55 (GMTKN55) dataset is a collection of 55 datasets aimed at probing the accuracy of quantum mechanics (QM) methods across a variety of chemical problems, ranging from reaction energies and electronic properties to non-covalent interaction energies and conformational properties. The dataset consists of 55 individual subsets with a total of 1505 relative energies based on 2462 single-point calculations. The high-level reference energies for the molecules in GMTKN55 may be best-estimates calculated using a range of extrapolative protocols based on CCSD and CCSD(T) calculations collected from several different sources.

The performance of QM methods on GMTKN55 can be presented via aggregated scores based on weighting of the mean absolute deviation to a reference, the WTMAD-1 or WTMAD-2 scores, with the difference between the two being the relative weighting of the individual subsets.

For the subsets in OrbNet training data, the WTMAD-1 and WTMAD-2 score are 5.97 and 9.84, compared to the high-level reference energies. Considering all subsets where the elements and spin-states are present in the training set, but where the chemical space is not necessarily covered by the OrbNet training data (for example transition states, inorganic systems, etc), WTMAD-1 and WTMAD-2 are not substantially increased, at 7.19 and 9.85 against the high-level reference energies.

When the weighted scores are calculated against ωB97X-D3/def2-TZVP reference energies (the same method used to generate the OrbNet training data), the WTMAD-1 and WTMAD-2 scores are 3.67 and 6.37, respectively. For the version of OrbNet Denali trained on 10% of the data, the WTMAD-1 and WTMAD-2 scores are 7.77 and 12.16, respectively, against the ωB97X-D3/def2-TZVP reference, demonstrating positive effects of increasing the dataset size. The WTMAD-1 and WTMAD-2 between ωB97X-D3/def2-TZVP and the high-level reference energies are 3.67 and 6.37, respectively, which in some sense constitutes an upper bound for the accuracy versus high-level reference energies of an OrbNet model trained on ωB97X-D3/def2-TZVP data.

In comparison, the popular semi-low-cost DFT method B97-3c has WTMAD-1 and WTMAD-2 values for GMTKN55 of 5.76 and 10.22, respectively, compared to the high-level references, very close to the OrbNet scores. For this dataset, OrbNet is roughly 100 times faster than B97-3c. Another low-cost QM method is the series of GFNn-xTB (n∈{0, 1, 2}) methods. For these methods, the WTMAD-1 values are 45.9, 20.9, and 15.4 for GFN0-xTB, GFN1-xTB and GFN2-xTB respectively, with the same series of WTMAD-2 numbers being 75.8, 35.9, and 27.4. GFN1-xTB is the baseline method used to generate the input for OrbNet Denali, and that, despite of the relatively poor performance across GMTKN55, OrbNet yields DFT-quality energy predictions.

For the machine learning potentials ANI-1ccx and ANI-2x, the WTMAD-n scores over the subsets that only contain neutral singlet molecules with the elements that are covered by the individual methods can be calculated. For the ANI model that is parametrized against CCSD(T) reference data, namely ANI-1cc, the WTMAD-1 and WTMAD-2 values are 15.5 and 24.2, respectively. For the ANI-2x model which, similarly to OrbNet Denali, is parametrized on DFT-level data, the WTMAD-1 and WTMAD-2 are 14.2 and 23.9, respectively.

In terms of coverage of common chemistry problems to which a general-purpose machine learning potential can be applied, OrbNet Denali can provide a broad coverage of GMTKN55. OrbNet Denali covers 37 out of the 55 subsets, due to the OrbNet training set not covering the elements He, Be, and Al as well as a few heavy metals, and well as spin states other than singlets, for example used to calculate ionization potentials and electron affinities. When extrapolating out of the training distribution to these other subsets, OrbNet Denali provides reasonable, but less accurate, results due to its basis in GFN1-xTB. The corresponding numbers for ANI-1ccx and ANI-2x are 14 and 20, respectively. ANI-1ccx covers only neutral, singlet molecules with the elements H, C, N, and 0, while ANI-2x extends this coverage to the elements F, CI, and S. The family of GFNn-xTB methods have been parametrized on data up to Radon (Z=87) and also handles systems with odd numbers of electrons, and is therefore able to cover GMTKN55.

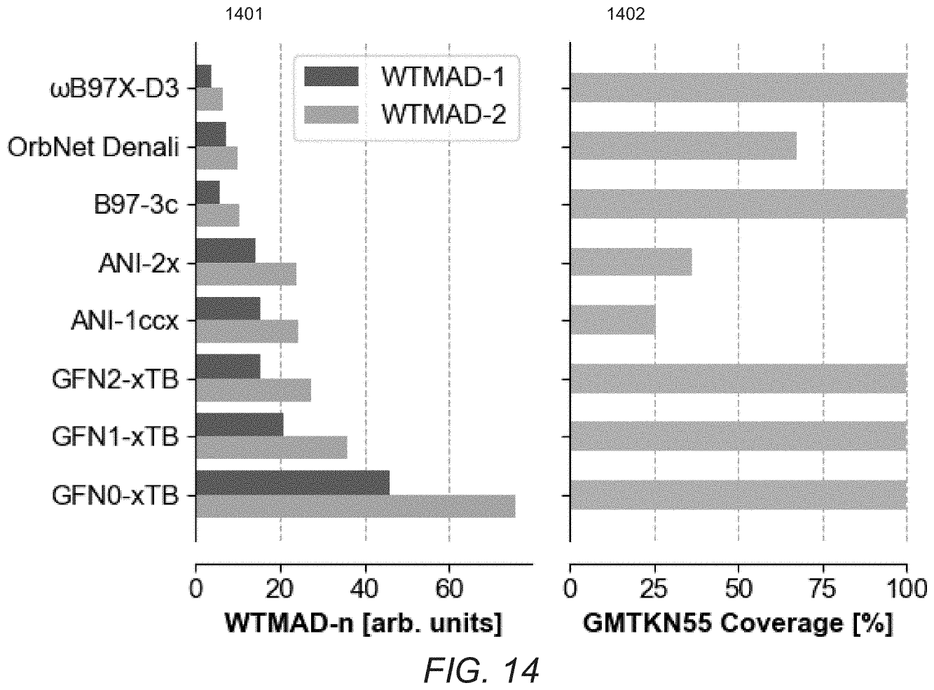

A graphical overview of WTMAD-n values and the coverage of GMTKN55 subsets for each method in accordance with an embodiment of the invention is illustrated in FIG. 14. Statistics over the accuracy and coverage of the GMTKN55 dataset is shown for a selection of methods, sorted by WTMAD-2 scores, relative to reference high-level estimates. The aggregated WTMAD-1 and WTMAD-2 metrics in arbitrary units, calculated on subsets, that are covered by each method are shown in 1401. The percentage of GMTKN55 subsets consisting of molecules with elements, charged states, and spin states that are allowed within each model are shown in 1402. The def2-TZVP basis set is used for the ωB97X-D3 calculations.

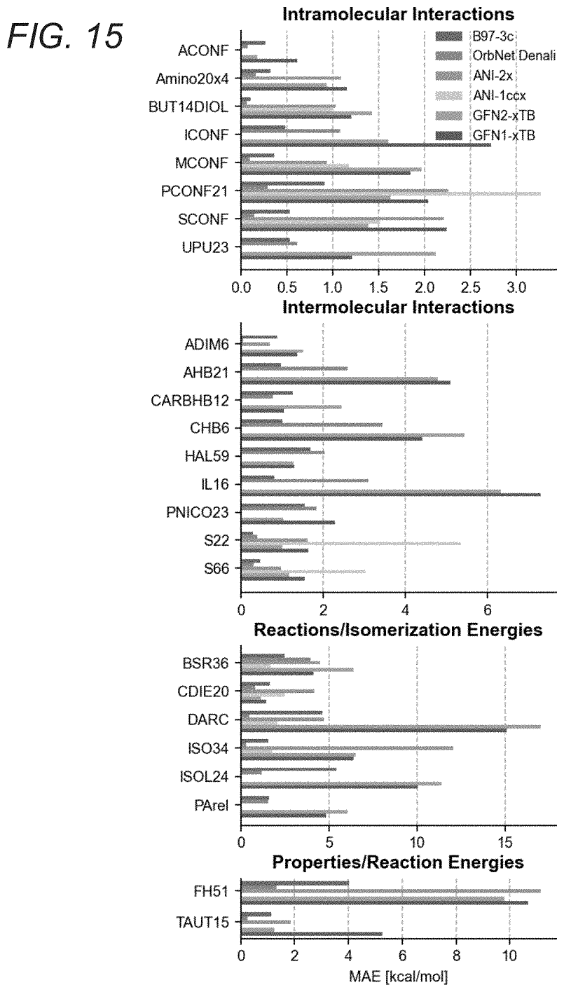

MAE in kcal/mol for the subsets of the GMTKN55 covered by OrbNet Denali training data relative to ωB97X-D3/def2-TZVP in accordance with an embodiment of the invention is illustrated in FIG. 15. For ANI-1ccx and ANI-2x, values are left out for those subsets that contain elements or charge states that are not allowed within those models.

### Example 7: Conformer Scoring with OrbNet of SAAO Features

Accurate determination of the ensemble of thermally accessible conformers is key to modelling molecules. Example 7 includes results for a benchmark for conformer energetics. This benchmark contains up to ten poses for each of ˜700 drug-like molecules. Each molecule is comprised of elements from the set C, H, N, O, S, Cl, F, P, Br, I, and contains between nine and fifty heavy atoms with a total charge between −1 and +2.

Accuracy in this benchmark for a given method is reported as median R² and is determined as follows. For every molecule, the correlation coefficient (R²) is computed between the conformer energies of that molecule and the reference (DLPNO-CCSD(T)) energies. The median is then taken over the set of R² values corresponding to all molecules in the benchmark.

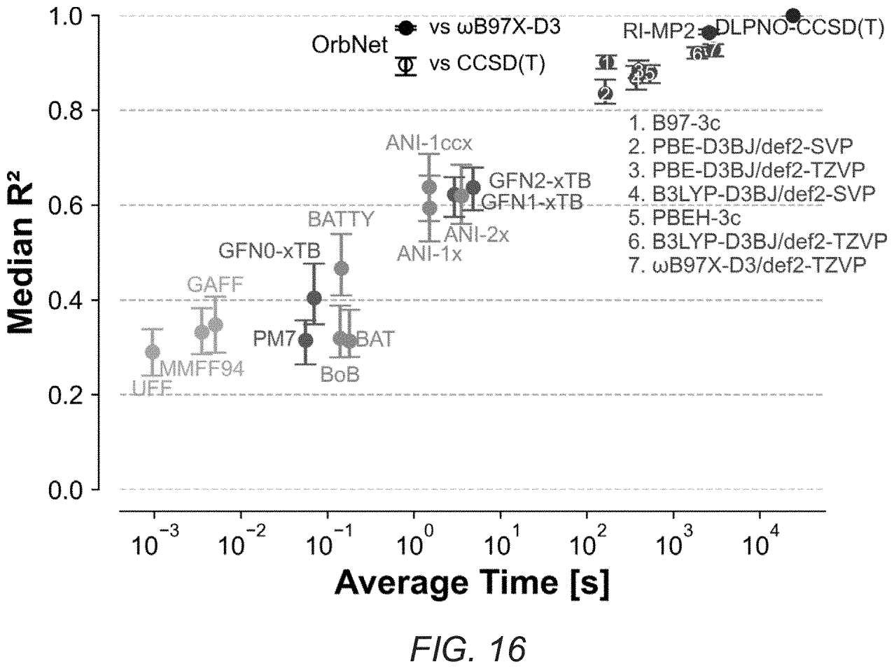

A comparison between computational cost and the resulting accuracy for a number of methods for the Hutchison conformer benchmark set in accordance with an embodiment of the invention is illustrated in FIG. 16. The comparison of OrbNet Denali to a representative sample of computational chemistry methods, including force fields, machine learning, semi-empirical, density functional theory, and wavefunction theory is shown. The horizontal axis shows the average time of a single conformer energy computation, while the vertical axis denotes the median R² correlation coefficient for the molecules in the dataset. Error bars denote the 95% confidence interval for this number and are obtained through bootstrapping. The median correlation coefficient versus DLPNO-CCSD(T) is shown for all methods (filled circles, open, white circle for OrbNet). Additionally, the median correlation coefficient versus ωB97X-D3/def2-TZVP reference energies is shown for OrbNet (filled, black circle). This reference corresponds to the level of theory used to train the model.

For methods other than OrbNet, a strong correlation can be observed between accuracy and the logarithm of the average execution time of the method. OrbNet Denali instead provides a median R² of about 0.90±0.02 versus the reference DLPNO-CCSD(T) at an average execution time of approximately one second per molecule. The uncertainty refers to the 95% confidence interval and is obtained by bootstrapping the dataset. GFN1-xTB, the method used to generate input for OrbNet, provides a median R² of 0.62±0.04 with a similar execution time to OrbNet. The median R² between OrbNet and ωB97X-D3/def2-TZVP, the same method used to generate the training data for OrbNet, is 0.973±0.004, highlighting that OrbNet is able to learn its underlying method to high accuracy. Compared to the ωB97X-D3/def2-TZVP level of theory which provides a similar accuracy to DLPLO-CCSD(T) with a median R² of 0.92±0.02, OrbNet results in a more than thousand-fold speedup. This number also serves as an upper bound for the accuracy of a model trained on ωB97X-D3/def2-TZVP data, and suggests that to increase the median R² for OrbNet compared to DLPNO-CCSD(T), it may be necessary to train on data that exceeds the accuracy of DFT.

### Example 8: Non-Covalent Interactions (S66x10) with OrbNet of SAAO Features

A standard benchmark for the accuracy of non-covalent interaction can be the S66x10 benchmark set. This dataset consists of 66 different molecular dimers and their equilibrium geometries, along with 9 additional displacements along the center-of-mass axis and corresponding CCSD(T)/CBS extrapolated binding energies.

For OrbNet Denali, the MAE and RMSE to CCSD(T)/CBS are 0.75 and 1.01 kcal/mol respectively. These numbers are close to the MAE and RMSE for the method used to generate the training data, ωB97X-D3/def2-TZVP, at 0.70 and 0.91 kcal/mol. Comparing OrbNet Denali to ωB97X-D3/def2-TZVP, smaller MAE and RMSE values can be found at 0.46 and 0.65 kcal/mol, respectively. For OrbNet Denali, trained on 10% of the data, these numbers increase to 0.67 and 0.85, respectively, suggesting that the increased training data size can be beneficial, but also that it may be impossible for the model to substantially surpass the accuracy of the training data. The numbers referred to in this section are summarized in Table 7. For rows marked with an asterisk (*), OrbNet predictions are compared to binding energies calculated at the ωB97X-D3/def2-TZVP level. The latter reference corresponds to the same method used to generate the training data for OrbNet Denali.

TABLE 7
The MAE and RMSE binding energies for the S66x10
benchmark versus CCSD(T)/CBS reference binding energies.

| | MAE | RMSE |
|---|---|---|
| Model | [kcal/mol] | [kcal/mol] |
| OrbNet Denali | 0.75 | 1.01 |
| ωB97X-D3/def2-TZVP | 0.70 | 0.91 |
| B97-3c | 0.49 | 0.64 |
| GFN2-xTB | 1.19 | 1.75 |
| GFN1-xTB | 1.35 | 1.87 |
| GFN0-xTB | 2.35 | 3.10 |
| ANI-2x | 1.44 | 1.96 |
| ANI-1ccx | 2.82 | 3.95 |
| *OrbNet Denali | 0.46 | 0.65 |
| *OrbNet Denali 10% | 0.67 | 0.85 |

### Example 9: Torsional Profiles of Druglike Molecules with OrbNet of SAAO Features

A benchmark for empirical potentials can be the accuracy to which torsional profiles can be reproduced. The TorsionNet500 benchmark compiles torsional profiles of 500 chemically diverse fragments containing the elements H, C, N, O, F, S, and Cl. For these torsion profiles, reference energies at the ωB97X-D3/def2-TZVP level are computed, corresponding to the level of theory used to train OrbNet Denali. Some embodiments benchmark the performance of OrbNet Denali by comparing several different measures of accuracy. An overview can be found in Table 8. In Table 8, The reference energies are calculated at the ωB97X-D3/def2-TZVP level of theory, except for rows marked with an asterisk (*), benchmarked against a B3LYP/6-31G* reference. For a number of methods, the following statistics are shown: the percentage of the 500 torsion profiles for which the Pearson correlation coefficient (R) is greater than 0.9, the average Pearson R over the torsion profiles, the MAE and RMSE of the relative energies of the torsion profiles and, lastly, the percentage of torsion profiles, where the local minimum angle of the reference profile corresponds to a point within 20° in the test profiles which is also no more than 1 kcal/mol from the global minimum.

The first measure is the number of torsion profiles where the Pearson correlation coefficient (R) between the reference energies and the predicted energies is greater than 0.9. For OrbNet Denali, this can be true for about 99.4% of the profiles, while for OrbNet Denali (10%), the corresponding number is about 98.8\%, with average Pearson R values of 0.995 and 0.988, respectively. Second, the average MAE and RMSE for the torsion profiles are 0.12 and 0.18 kcal/mol for the full OrbNet Denali models, and 0.23 and 0.34 kcal/mol for OrbNet Denali (10%). Finally, both OrbNet Denali models correctly predict the location of the global minimum to within 20° and its energy to within 1 kcal/mol for all 500 profiles. Embodiments provide that these results are achieved when the OrbNet Denali training set contains no torsion profiles.

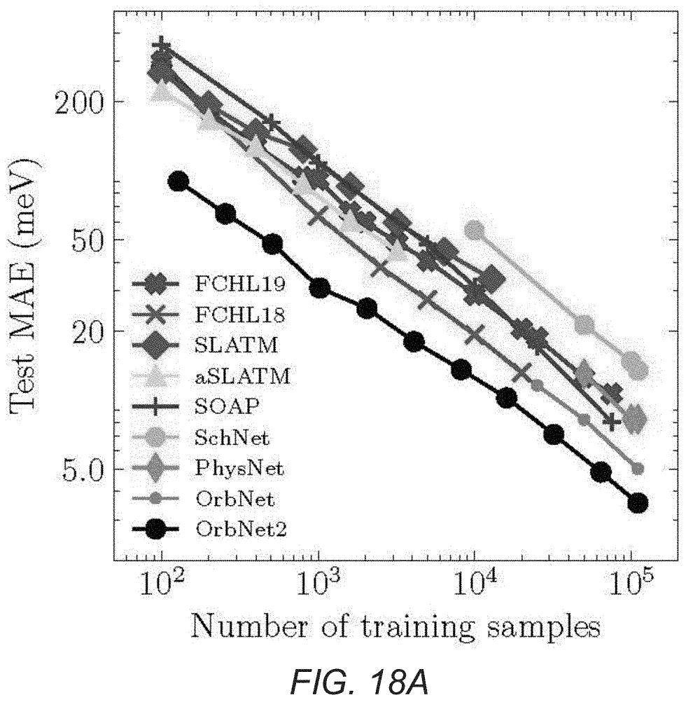

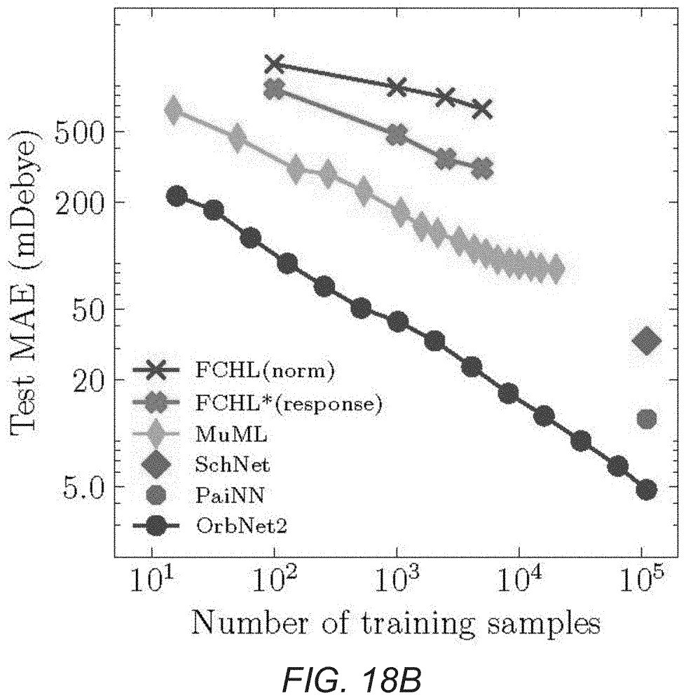

For OrbNet's baseline method, GFN1-xTB, the same figures are much lower with only 65.6% of the profiles having R>0.9, and with an average R value of 0.832, and the average MAE and RMSE 0.94 and 1.3 kcal/mol, while capturing a good minimum for 89.4% of the predicted profiles. 25 torsion energy profiles of OrbNet Denali versus GFN1-xTB stratified by the error of OrbNet Denali in accordance with an embodiment is illustrated in FIG. 18. FIG. 18 show torsional profiles of 25 druglike molecules from the TorionNet500 database, stratified to represent quintiles of the OrbNet error relative to the same torsional profiles calculated at the ωB97X-D3/def2-TZVP level of theory, shown as a reference (black). The same torsion profiles are shown for OrbNet's baseline method, GFN1-xTB (red). OrbNet Denali reproduces every point along every torsion within chemical accuracy of 1 kcal/mol in every case. For all but the 5 worst cases, the OrbNet Denali torsion profile is qualitatively identical to that of the reference method. On the other hand, GFN1-xTB shows large errors for the majority of torsion profiles. In some cases, the shape of the GFN1-xTB profile is qualitatively incorrect. Overall, this suggests that the accuracy of OrbNet is not solely due to the use of a good baseline method, but rather that OrbNet is able to capture the subtle differences correctly in the torsion profiles.

Torsion profiles calculated using other DFT method, B97-3c, are compared to the reference profiles. For B97-3c, the MAE and RMSE with regard to the ωB97X-D3 profile are 0.29 and 0.43 kcal/mol. These numbers may highlight that OrbNet Denali is almost three times closer to the DFT reference than the variation between these two DFT methods. OrbNet can therefore be considered on-par with DFT methods for this application.

OrbNet is also compared to the Merck Molecular Mechanics Force Field 94 (MMFF94) and the two ML-based methods, ANI-2x and TorsionNet. The MMFF94 force field is found to have the lowest accuracy of capturing the ωB97X-D3/def2-TZVP predicted minima, only finding the right minimum within the tolerance about 75.2% of the time, and with higher MAE and RMSE across the torsion profiles, at 1.4 kcal/mol and 5.2 kcal/mol, respectively. For ANI-2x, a low-energy minimum is captured within the 20 tolerance with a 91.8% success rate, compared to the ωB97X-D3/def2-TZVP reference torsion profiles, which is better than MMFF94, GFN0-xTB and GFN1-xTB. ANI-2x may have better accuracy at finding the low-energy minima correctly, but it comes out with a larger MAE and RMSE than GFN0-xTB and GFN1-xTB, maybe due to underestimation of the rotational barriers.

In addition to numbers, some embodiments highlight the benchmarks that compares ANI-2x and TorsionNet on the same structures, but against B3LYP/6-31G(d) single points energies. ANI-2x may be parametrized against ωB97X/6-31G(d) reference data, while TorsionNet is parametrized against B3LYP/6-31G(d) reference data, so it may be possible that the reference data provides a fairer reference for ANI-2x. Against the B3LYP/6-31G(d) reference, TorsionNet is able to locate the low-energy minima with about 83% success, and ANI-2x about 66% success. The MAE and RMSE for TorsionNet against its torsion profile calculated at its own reference level of theory are 0.7 and 1.3 kcal/mol respectively, while the MAE and RMSE for ANI-2x are 1.4 and 2.0 kcal/mol respectively, which is within 0.1 kcal/mol from the same values versus the ωB97X-D3/def2-TZVP reference.

TABLE 8
The performance of eight methods on the
TorsionNet500 benchmark set.

| | | | | | % local |
|---|---|---|---|---|---|
| | % | | | | minima |
| | profiles | | | | with |
| | with | Average | | | Δθ < 20° |
| | Pearson | profile | MAE | RMSE | and ΔE ≤ 1 |
| Model | R > 0.9 | Pearson R | [kcal/mol] | [kcal/mol] | kcal/mol |
| OrbNet | 99.4% | 0.995 | 0.12 | 0.18 | 100.0% |
| Denali | | | | | |
| OrbNet | 98.8% | 0.988 | 0.23 | 0.34 | 100.0% |
| Denali (10%) | | | | | |
| B97-3c | 97.4% | 0.985 | 0.29 | 0.43 | 100.0% |
| GFN2-xTB | 76.4% | 0.881 | 0.73 | 1.0 | 94.0% |
| GFN1-xTB | 65.5% | 0.832 | 0.94 | 1.3 | 89.4% |
| GFN0-xTB | 52.6% | 0.748 | 1.2 | 1.7 | 85.0% |
| MMFF94 | 54.2% | 0.760 | 2.4 | 5.2 | 75.2% |
| ANI-2x | 73.2% | 0.899 | 1.3 | 1.9 | 91.8% |
| *ANI-1ccx | 54% | 0.75 | 1.4 | 2.0 | 66% |
| *TorsionNet | 79% | 0.91 | 0.7 | 1.3 | 83% |

### Example 10: Energy and Dipole Moment Prediction in QM9 Dataset with OrbNet for AO Based Features

Many embodiments implement OrbNet with AO based features in learning quantum-chemical properties including (but not limited to) single-point energies, forces, dipole moment, electron density, molecular orbital energies and thermal properties on various machine learning datasets. Several embodiments perform zero-shot generalization tests for OrbNet models pretrained on energies, against down-stream chemistry tasks that have been exploited to benchmark quantum-chemistry simulation methods. The same set of model hyperparameters are used in Examples 10-13.

In many embodiment, OrbNet processes outperform other methods by at least 150% on QM9 dataset, at least 114% on MD17 dataset, and at least 50-75% on electron densities. Beyond its learning efficiency, OrbNet trained on energies can achieve robust performance on various practical, down-stream chemistry tasks without any model fine-tuning. It offers an accuracy competitive to DFT methods with up to 3 orders of magnitude speedup.

Several embodiments implement OrbNet with AO based features in learning quantum-chemical properties including (but not limited to) energies and dipole moments on QM9 datasets. The QM9 dataset contains 134k small organic molecules with up to 9 heavy (CNOF) atoms in their equilibrium geometries, with scalar-valued chemical properties computed by DFT. Due to its simple chemical composition and multiple tasks, QM9 can be used to benchmark deep learning methods. Training on QM9 targets is carried out using 110,000 random samples as the training set and another 10,831 samples as the test set. OrbNet processes in accordance with several embodiments provide at least about 150% average decrease of MAE relative to other models on all 12 targets. In some embodiments, OrbNet can achieve qualitative improvements on dipole norm μ, electronic spatial extent [symbol] R² [symbol] , HOMO/LUMO energies and gap ϵ_{HOMO}, ϵ_{LUMO}, Δϵ, which are deeply rooted in the electronic structure in their formulations. Experiments are also performed on two representative targets, energy U₀ and dipole vector \vec{μ}. OrbNet outperforms deep learning methods and also pre-engineered approaches at different size of training data.

Table 9 lists prediction MAEs on QM9 targets for models trained on 110k samples. The best/second-best results on each task are marked in bold/by underline. OrbNet outperforms the second-best model (SphereNet) by 150% on average on all 12 targets.

FIGS. 18A-18B illustrate energy and dipole moment prediction with OrbNet of AO based features in accordance with an embodiment of the invention. FIG. 19A compares OrbNet to task-specific models and deep learning methods for energy U₀ in meV vector on QM9 dataset at different training data sizes. FIG. 19B compares OrbNet to task-specific models and deep learning methods for dipole moment \vec{μ} in mDebye vector on QM9 dataset at different training data sizes. OrbNet outperforms deep learning methods and pre-engineered approaches at different size of training data.

TABLE 9
Prediction MAEs on QM9 Dataset

| Target | Unit | SchNet | PhysNet | Cormorant | DimeNet++ | PaiNN | SphereNet | OrbNet |
|---|---|---|---|---|---|---|---|---|
| μ | mD | 33 | 53 | 38 | 29.7 | 12 | 26.9 | 6.3 |
| α | a₀³ | 0.235 | 0.062 | 0.085 | 0.044 | 0.045 | 0.047 | 0.036 |
| ∈_{HOMO} | meV | 41 | 34 | 32.9 | 24.6 | 27.6 | 23.6 | 9.9 |
| ∈_{LUMO} | meV | 34 | 24.7 | 38 | 19.5 | 20.4 | 18.9 | 12.7 |
| Δ∈ | meV | 63 | 42.5 | 38 | 32.6 | 45.7 | 32.3 | 17.3 |
| [symbol] R² [symbol] | a₀³ | 0.073 | 0.765 | 0.961 | 0.331 | 0.066 | 0.292 | 0.030 |
| ZPVE | meV | 1.7 | 1.4 | 2.0 | 1.2 | 1.3 | 1.1 | 1.1 |
| U₀ | meV | 14 | 8.2 | 22 | 6.3 | 5.9 | 6.3 | 3.5 |
| U | meV | 19 | 8.3 | 21 | 6.3 | 5.8 | 7.3 | 3.5 |
| H | meV | 14 | 8.4 | 21 | 6.5 | 6.0 | 6.4 | 3.5 |
| G | meV | 14 | 9.4 | 20 | 7.6 | 7.4 | 8.0 | 5.2 |
| c_{ν} | $\frac{cal}{molK}$ | 0.033 | 0.028 | 0.026 | 0.023 | 0.024 | 0.022 | 0.022 |
| std. MAE | % | 1.76 | 1.37 | 1.44 | 0.98 | 1.01 | 0.94 | 0.47 |
| log. MAE | — | −5.17 | −5.35 | −4.97 | −5.67 | −5.84 | −5.69 | −6.37 |

### Example 11: Energy and Force Prediction in MD17 Dataset with OrbNet for AO Based Features

Many embodiments implement OrbNet with AO based features in learning quantum-chemical properties including (but not limited to) energies and forces on MD17 datasets. The MD17 dataset contains energy and force labels from molecular dynamics trajectories of eight small organic molecules, and can be used to benchmark ML methods for modelling a single instance of potential energy surface. OrbNet is trained on energies and forces of 1000 geometries of each molecule and tested on another 1000 molecules, using reported dataset splits and revised labels. OrbNet can achieve over 110% average improvements on both energy and force predictions, when compared to hand-engineered features combined with kernel regressions kernel methods and graph neural networks. Uncertainties are estimated as the standard deviation of MAE on the test set for 3 independently trained models.

Table 10 lists prediction of MAEs on MD17 energies (in kcal/mol) and forces (in kcal/mol/Å) for models trained on 1000 samples. On average, OrbNet outperforms other energy model (i.e., FCHL19/GPR) by at least 138% and other force model (i.e., NequlP) by at least 114%.

TABLE 10
Prediction MAEs on MD17 energies (in kcal/mol) and forces (in kcal/mol/Å).

| | | Kernel Methods | | | Neural Networks | | |
|---|---|---|---|---|---|---|---|
| Molecule | | sGDML | FCHL19 | DimeNet | NequllP | PaiNN | OrbNet |
| Aspirin | Energy | 0.19 | 0.144 | 0.204 | — | 0.159 | 0.056 ± 0.002 |
| | Forces | 0.68 | 0.481 | 0.499 | 0.348 | 0.371 | 0.181 ± 0.005 |
| Ethanol | Energy | 0.07 | 0.021 | 0.064 | — | 0.063 | 0.017 ± 0.000 |
| | Forces | 0.33 | 0.144 | 0.230 | 0.208 | 0.230 | 0.096 ± 0.002 |
| Malonaldehyde | Energy | 0.10 | 0.035 | 0.104 | — | 0.091 | 0.029 ± 0.001 |
| | Forces | 0.41 | 0.237 | 0.383 | 0.337 | 0.319 | 0.163 ± 0.005 |
| Naphthalene | Energy | 0.12 | 0.028 | 0.122 | — | 0.117 | 0.010 ± 0.000 |
| | Forces | 0.11 | 0.150 | 0.215 | 0.096 | 0.083 | 0.055 ± 0.001 |
| Salicylic Acid | Energy | 0.12 | 0.041 | 0.134 | — | 0.114 | 0.017 ± 0.000 |
| | Forces | 0.28 | 0.220 | 0.374 | 0.238 | 0.209 | 0.095 ± 0.001 |
| Toluene | Energy | 0.10 | 0.039 | 0.102 | — | 0.097 | 0.013 ± 0.001 |
| | Forces | 0.14 | 0.204 | 0.216 | 0.101 | 0.102 | 0.067 ± 0.001 |
| Uracil | Energy | 0.11 | 0.013 | 0.115 | — | 0.104 | 0.013 ± 0.001 |
| | Forces | 0.24 | 0.097 | 0.301 | 0.172 | 0.140 | 0.087 ± 0.005 |
| Benzene | Energy | 0.10 | 0.008 | 0.078 | — | — | 0.002 ± 0.000 |
| | Forces | 0.06 | 0.060 | 0.187 | 0.053 | — | 0.016 ± 0.001 |

### Example 12: Electron Density Prediction with OrbNet for AO Based Features

Many embodiments implement OrbNet with AO based features in learning quantum-chemical properties including (but not limited to) electron density on BfDB-SSI and QM9 datasets. Several embodiments provide prediction of the electron density of molecules ρ(\vec{r}): [symbol] ³→ [symbol] which plays an essential role in both the theoretical formulation and practical construction of DFT. O(3) equivariance of OrbNet enables to efficiently learn ρ(\vec{r}) in a compact atomic-orbital-like basis. Compared to two baselines that are specifically developed for learning ρ(\hat{r}), OrbNet2 achieves about 50-75% reduction in mean L−1 density error

$${∈}_{ρ} : = \frac{\int \vert ρ ( \vec{r} ) - {ρ}_{θ} ( \vec{r} ) \vert d \vec{r}}{\int \vert ρ ( \vec{r} ) \vert d \vec{r}}$$

where ρ_{θ}(\vec{r}) denotes the model-predicted electron density. OrbNet is more efficient at training compared to SA-GPR which has a cubic training time complexity, and at inference compared to DeepDFT which requires evaluating part of the neural network at each grid point \vec{r}.

Table 11 lists electron charge density learning statistics. OrbNet outperforms baselines by at least 52% on BfDB-SSI and at least 75% on QM9 in ϵₚ with significant training and inference efficiency advantages.

TABLE 11
Electron charge density learning statistics.

| | | Mean test error ϵ_{ρ} (%) | | |
|---|---|---|---|---|
| Dataset | Training Samples | SA-GPR | DeepDFT | OrbNet |
| BfDB-SSI | 2000 | 0.29 | — | 0.191 ± 0.003 |
| QM9 | 123835 | — | 0.36 | 0.206 ± 0.001 |

### Example 13: Down-Stream Chemistry Tasks with OrbNet for AO Based Features

Many embodiments provide the performance of OrbNet on metrics interested by chemists. In several embodiments, OrbNet2 model can be trained on the DFT energies of 237k samples with broad chemical space coverage and non-equilibrium geometries, and without any model fine-tuning, directly apply it to down-stream tasks commonly used to benchmark quantum-chemistry simulation methods. In this zero-shot setting, pretrained OrbNet model achieves accuracies similar and/or better than a DFT functional while being around at least 200 times faster (more than 1000 times faster if running OrbNet on GPUs), and is significantly better than representative semi-empirical quantum mechanics or machine learning methods which offer comparable speeds.

Table 12 lists benchmarking OrbNet against representative semi-empirical quantum mechanics (SEQM), machine learning (ML), and density functional theory (DFT) methods on down-steam tasks.

TABLE 12
Benchmarking OrbNet against representative semi-empirical quantum
mechanics (SEQM), machine learning (ML), and density functional
theory (DFT) methods on down-steam tasks.

| | | SEQM | ML | DFT | |
|---|---|---|---|---|---|
| Task/benchmark | Metric | GFN2-xTB | ANI-2x | B97-3c | OrbNet |
| Speed | Relative time-to-solution ↓ | ~5 | ~4 | ~200 | ~1 |
| Data efficiency | Training dataset size ↓ | — | 8.9M E + F | — | 236K E only |
| Drug chemistry | Sample coverage rate ↑ | 100% | 81% | 100% | 100% |
| coverage | | | | | |
| General chemistry | Subset coverage rate ↑ | 100% | 36% | 100% | 67% |
| coverage | | | | | |
| Conformer ranking | R²[DLPNO] ↑ | 0.63 ± 0.04 | 0.63 ± 0.06 | 0.90 ± 0.01 | 0.89 ± 0.02 |
| Torsion profiles | MAE (kcal/mol) ↓ | 0.73 ± 0.01 | 0.90 ± 0.01 | 0.29 ± 0.00 | 0.17 ± 0.00 |
| Reaction energies | WTMAD-2 ↓ | 36.1 ± 3.8 | 19.2 ± 10.1 | 14.6 ± 1.6 | 14.6 ± 4.3 |
| Intra-molecular | WTMAD-2 ↓ | 25.1 ± 2.4 | 29.6 ± 5.8 | 8.6 ± 0.9 | 10.3 ± 1.7 |
| interactions | | | | | |
| Geometry | RMSD (Å) ↓ | 0.21 ± 0.08 | NaN | 0.06 ± 0.01 | 0.06 ± 0.01 |
| optimizations | | 0.60 ± 0.06 | NaN | 0.51 ± 0.07 | 0.18 ± 0.02 |
| | Extrapolative benchmarks | | | | |
| Reaction barriers | WTMAD-2 ↓ | 25.8 ± 3.7 | 41.3 ± 23.2 | 11.8 ± 1.0 | 20.3 ± 3.2 |
| Intra-molecular | WTMAD-2 ↓ | 10.8 ± 1.2 | 44.8 ± 10.9 | 11.1 ± 1.2 | 19.3 ± 3.4 |
| interactions | | | | | |

### DOCTRINE OF EQUIVALENTS

As can be inferred from the above discussion, the above-mentioned concepts can be implemented in a variety of arrangements in accordance with embodiments of the invention. Accordingly, although the present invention has been described in certain specific aspects, many additional modifications and variations would be apparent to those skilled in the art. It is therefore to be understood that the present invention may be practiced otherwise than specifically described. Thus, embodiments of the present invention should be considered in all respects as illustrative and not restrictive.

What is claimed is:

1. A method of synthesizing a molecule comprising,
obtaining a set of atomic orbitals for a molecular system using a computer system;
generating a set of atomic-orbital-based features based upon the set of atomic orbitals of the molecular system using the computer system;
determining at least one molecular system property based on the set of features using an atomic-orbital-based machine learning (OrbNet) model implemented on the computer system; and
when the determined at least one molecular system property satisfies at least one criterion by the computer system, synthesizing the molecular system.

2. The method of claim 1, wherein the set of atomic-orbital-based features comprises an attributed graph representation of atomic-orbital-based features.

3. The method of claim 2, wherein a node feature of the attributed graph representation corresponds to a diagonal atomic orbital block and an edge feature of the attributed graph representation corresponds to an off-diagonal atomic orbital block.

4. The method of claim 1, wherein the set of atomic-orbitals comprises symmetry-adapted-atomic-orbitals (SAAOs) and the set of atomic-orbital-based features comprises a set of features based on atomic-orbitals, a set of features based on SAAOs, derivatives of a set of features based on atomic-orbitals or derivatives of a set of features based on SAAOs.

5. The method of claim 1, wherein:
the molecular system is one of a plurality of candidate molecular systems; and
determining when the determined at least one molecular system property satisfies at least one criterion further comprises:
generating a set of atomic-orbital-based features based upon sets of atomic orbitals for each of the candidate molecular systems;
determining at least one molecular system property for each of the candidate molecular systems based on the set of atomic-orbital-based features of each of the candidate molecular systems using the OrbNet model;
screening the candidate molecular systems based upon the at least one molecular system property determined for each of the candidate molecular systems; and
identifying the molecular system based upon the screening.

6. The method of claim 1, further comprising training the OrbNet model to learn relationships between sets of atomic-orbital-based features and molecular system properties using a training dataset describing a plurality of molecular systems and their molecular system properties.

7. The method of claim 6, wherein training the OrbNet model to learn relationships between sets of atomic-orbital-based features and molecular system properties further comprises:
obtaining a set of atomic orbitals for each molecular system in the training dataset of molecular systems; and
obtaining a set of atomic-orbital-based features based upon the set of atomic orbitals.

8. The method of claim 7 further comprises:
obtaining a set of symmetry-adapted-atomic-orbitals for each molecular system in the training dataset of molecular systems by constructing rotationally invariant symmetry-adapted atomic orbital basis sets; and
obtaining a set of symmetry-adapted-atomic-orbital-based features based upon at least the symmetry-adapted-atomic-orbitals.

9. The method of claim 7, wherein obtaining the set of atomic orbitals comprises calculating one mean-field electronic structure selected from the group consisting of Hartree-Fock theory, density functional theory, and a semi-empirical method, and obtaining the set of atomic-orbital-based features comprises calculating one mean-field electronic structure selected from the group consisting of Hartree-Fock theory, density functional theory, and a semi-empirical method.

10. The method of claim 7, wherein obtaining the set of atomic orbitals comprises parameterizing at least one quantum mechanical operator appeared in the formulation of an electronic structure method selected from the group consisting of Hartree-Fock theory, density functional theory, and a semi-empirical method by a neural network, and obtaining the set of atomic-orbital-based features comprises parameterizing at least one quantum mechanical operator appeared in the formulation of an electronic structure method selected from the group consisting of Hartree-Fock theory, density functional theory, and a semi-empirical method by a neural network.

11. The method of claim 10, wherein the neural network comprises a graph neural network, wherein at least one node of the graph neural network corresponds to at least one atom, and at least one edge of the graph neural network corresponds to at least one interatomic interaction.

12. The method of claim 10, wherein training the OrbNet model and the neural network takes place simultaneously.

13. The method of claim 8, wherein determining the symmetry-adapted-atomic-orbitals comprises diagonalizing at least one diagonal density-matrix block.

14. The method of claim 6, wherein training the OrbNet model comprises graph neural network.

15. The method of claim 14, wherein the graph neural network comprises at least one message passing layer and at least one decoding layer.

16. The method of claim 1, wherein the molecular system comprises at least one of atoms, molecular bonds, and molecules formed by atoms and molecular bonds.

17. The method of claim 1, wherein the set of features includes atomic-orbital-based features comprising a physical operator.

18. The method of claim 17, wherein the atomic-orbital-based features further comprise at least one feature selected from the group consisting of:
elements from a Fock matrix,
elements from a Coulomb matrix,
elements from a Hartree-Fock matrix,
elements from a density matrix;
elements from a core Hamiltonian matrix; and
elements from an overlap matrix.

19. The method of claim 1, wherein the at least one molecular system property comprises at least one property selected from the group consisting of quantum correlation energy, conformer energy, mean-field energy, single point energy, learning energy, molecular orbital energy, potential energy surface, force, inter-atomic force, vibrational frequency, dipole moment, electronic density, response property, thermal property, excited state energy, excited state force, linear-response excited state energy, linear-response excited state force, and spectrum.

20. The method of claim 1, wherein the synthesized molecular system comprises at least one molecule selected from the group consisting of a catalyst, an enzyme, a pharmaceutical, a protein, an antibody, a surface coating, a nanomaterial, a semiconductor, and an organic material.

21. A method of screening a set of candidate molecular systems comprising:
obtaining a set of atomic orbitals for a plurality of candidate molecular systems using a computer system;
generating a set of atomic-orbital-based features for each candidate molecular system based upon sets of atomic orbitals for each of the candidate molecular systems using the computer system;
determining at least one molecular system property for each of the candidate molecular systems based on the set of atomic-orbital-based features of each of the candidate molecular systems using an atomic-orbital-based machine learning (OrbNet) model implemented on the computer system;
screening the candidate molecular systems to identify at least one molecular system possessing at least one molecular system property that satisfies at least one criterion based upon the at least one molecular system property determined for each of the candidate molecular systems using the computer system; and
generating a report describing the at least one molecular system identified during the screening of the candidate molecular systems using the computer system.

22. A method of synthesizing a molecular system using an inverse molecule design process comprising:
searching for a set of atomic-orbital-based features having at least one molecular system property predicted by an atomic-orbital-based machine learning (OrbNet) model that satisfies at least one criterion using a computer system, where the OrbNet model is trained to receive a set of features of a molecular system and output an estimate of at least one molecular system property;
mapping a located set of atomic-orbital-based features to an identified molecular system using a feature-to-structure map using the computer system, where the feature-to-structure map is trained to map a set of atomic-orbital-based features to a corresponding molecular structure;
screening the identified molecular system based upon at least one screening criterion using the computer system; and
when the identified molecular system satisfies the at least one screening criterion, synthesizing the identified molecular system.

23. The method of claim 22, wherein searching for a set of atomic-orbital-based features having at least one molecular system property predicted by the OrbNet model that satisfies at least one criterion further comprises using at least one generative model to generate candidate sets of features.

24. The method of claim 23, wherein the generative model comprises a graph neural network.

25. A method of training an atomic-orbital-based machine learning (OrbNet) model to predict at least one molecular system property from a set of atomic orbitals for a molecular system comprising:
obtaining a training dataset of molecular systems and their molecular system properties using a computer system;
generating a set of atomic-orbital-based features for each molecular system in the training dataset based upon a set of atomic orbitals for each of the candidate molecular systems using the computer system;
training a ML model to learn relationships between the set of atomic-orbital-based features of each molecular system in the training dataset and the molecular system properties of each of the molecular systems in the training dataset using the computer system; and
utilizing the OrbNet model to predict at least one molecular system property for a specific molecular system based upon a set of atomic-orbital-based features generated for the specific molecular system based upon a set of atomic orbitals for the specific molecular system.

26. The method of claim 25, wherein obtaining a training dataset of molecular systems and their molecular system properties further comprises:
generating a set of atomic-orbital-based features for the specific molecular system based upon a set of atomic orbitals for the specific molecular system using the computer system;
retrieving atomic-orbital-based features from a database based upon proximity between a retrieved atomic-orbital-based feature and an atomic-orbital-based feature from the set of atomic-orbital-based features for the specific molecular system; and
forming the training dataset using the retrieved molecular systems.

27. The method of claim 25, wherein training the OrbNet model to learn relationships between the sets of atomic-orbital-based features of each molecular system in the training dataset and the molecular system properties of each of the molecular systems in the training dataset further comprises utilizing a transfer learning process to train an OrbNet model previously trained to determine the relationship between an atomic-orbital-based features of a molecular system and a different set of molecular system properties.

28. The method of claim 25, wherein training the OrbNet model to learn relationships between the sets of atomic-orbital-based features of each molecular system in the training dataset and the molecular system properties of each of the molecular systems in the training dataset further comprises utilizing an online learning process to update a previously trained OrbNet model.
